(19) United States
(12) Patent Application Publication
(10) Pub. No.: US 2025/0325766 A1
(43) Pub. Date: Oct. 23, 2025

(54) A PATIENT INTERFACE AND A RESPIRATORY APPARATUS

(71) Applicant: Fisher & Paykel Healthcare Limited, Auckland (NZ)

(72) Inventors: Anton Kim GULLEY, Auckland (NZ); David Martin RUSSELL, Auckland (NZ); Philip Ian ROWE, Auckland (NZ); Francis George BRIDGE, Auckland (NZ); Brent Ian LAING, Auckland (NZ); Kylie Joy CLAMPITT, Auckland (NZ); Hazel Sarah GLASGOW, Auckland (NZ); Jason Allan KLENNER, Auckland (NZ)

(21) Appl. No.: 18/838,566

(22) PCT Filed: Feb. 28, 2023
(86) PCT No.: PCT/IB2023/051829

§ 371 Date: Aug. 14, 2024

Related U.S. Application Data
(60) Provisional application No. 63/268,691, filed on Feb. 28, 2022.
(60) Provisional application No. 63/367,891, filed on Jul. 7, 2022.

(51) Int. Cl. A61M 16/00; A61M 16/06

(52) U.S. Cl. CPC A61M 16/024; A61M 16/0003; A61M 16/0672; A61M 2205/587; A61M 2230/04; A61M 2230/20

(57) ABSTRACT

Disclosed is a nasal cannula interface accessory for attachment to a nasal cannula interface. The accessory comprising a sensor cavity, the sensor cavity configured to retain a sensor configured to measure at least one patient parameter and at least one securement feature, the at least one securement feature configured to connect the accessory to a nasal cannula interface (optionally to a strap of a nasal cannula interface).

### FIELD

The present disclosure generally relates to patient interfaces and respiratory apparatuses for respiratory support by providing a breathable gases flow to patients through a patient interface, and more particularly to accessories and/or components with sensors on or near the patient interface of a respiratory apparatus.

### BACKGROUND

When providing respiratory support to a patient, it can be beneficial to monitor one or more patient parameters during the course of the therapy. In order to measure these patient parameters one or more patient sensors is used, such as a pulse oximeter, which can be used to determine blood oxygen saturation and heart rate. These parameters can be used individually or in conjunction with further parameters in making an assessment of the patient's health. Additionally, these parameters can be used to adjust one or more control parameters of the respiratory support system being used to provide respiratory support to the patient. This adjustment can be done manually by a clinician, or automatically by a controller of the respiratory support system, such as through feedback control. The parameters being adjusted can include any one or more of flow rate, pressure, temperature, humidity, dew point, oxygen concentration, and/or oxygen saturation.

### SUMMARY

In an aspect, there is provided a nasal cannula interface accessory for attachment to a nasal cannula interface, the accessory comprising: a sensor cavity, the sensor cavity configured to retain a sensor configured to measure at least one patient parameter, and at least one securement feature, the at least one securement feature configured to connect the accessory to a nasal cannula interface (optionally to a strap of a nasal cannula interface).

In some configurations, at least one securement feature is configured to releasably connect the accessory to a nasal cannula interface.

In some configurations, the sensor cavity is formed on a first face of the nasal cannula interface accessory.

In some configurations, the accessory comprises a wire cavity, the wire cavity configured to provide a pathway for one or more wires to the sensor cavity.

In some configurations, each of the one or more wires comprises, a cable, a cord, a lead, or any other electrically conductive material.

In some configurations, the wire cavity is formed on the same face, and/or a different face as the sensor cavity of the nasal cannula interface accessory.

In some configurations, the wire cavity comprises a plurality of openings on different faces of the accessory (optionally adjacent faces).

In some configurations, the wire cavity is formed adjacent to the sensor cavity.

In another aspect there is provided a nasal cannula interface accessory for attachment to a nasal cannula interface, the accessory comprising:
a sensor cavity configured to retain a sensor configured to measure at least one patient parameter,
a wire cavity, the wire cavity configured to provide a pathway for one or more wires to the sensor cavity, and
a pair of arms, the pair of arms extending from the cannula interface accessory, the pair of arms extending towards each other and/or towards a center of the accessory, the pair of arms configured to releasably connect the accessory to a nasal cannula interface (optionally to a strap of a nasal cannula interface).

In another aspect there is provided a nasal cannula interface accessory for attachment to a nasal cannula interface, the accessory comprising:
a sensor cavity configured to retain a sensor configured to measure at least one patient parameter,
a wire cavity, the wire cavity configured to provide a pathway for one or more wires to the sensor cavity, and
a clip, the clip configured to be connected to the accessory, the clip comprising a clip arm connected to the accessory via a biasing element, the clip configured to secure the nasal cannula interface accessory to a nasal cannula interface (optionally to a strap of a nasal cannula interface).

In some configurations, the biasing element comprises a hinge, a spring.

In some configurations, the sensor cavity is one or more of: square, rectangular and/or circular.

In some configurations, the sensor cavity has substantially rounded edges and vertices.

In some configurations, the sensor cavity is arranged to orient the sensor to be in contact with a patient.

In some configurations, the sensor cavity is arranged to orient the sensor such that a transducer of the sensor faces towards the patient.

In some configurations, the sensor comprises a light transducer (optionally an infrared transducer, or a red-light transducer) and/or a light source (optionally an infrared source, or a red-light source).

In some configurations, the sensor cavity is arranged to orient the sensor such that the light transducer and/or light source faces towards the patient.

In some configurations, the sensor cavity comprises an opening on a face of the accessory which is configured to face a patient in use.

In some configurations, the wire cavity extends from an external face to the sensor cavity.

In some configurations, the wire cavity extends from a face of the accessory to the sensor cavity.

In some configurations, the wire cavity comprises an opening on a face of the accessory which is configured to face a patient in use.

In some configurations, the wire cavity is a slot.

In some configurations, the wire cavity is one or more of: square, rectangular and/or circular.

In some configurations, the sensor is a patient sensor, and the patient parameter is a physiological parameter.

In some configurations, the patient parameter is a measure of blood oxygenation of the patient.

In some configurations, the accessory is attachable to the interface at a plurality of interface attachment locations.

In some configurations, the accessory is configured to be adjustable between the plurality of interface attachment locations.

In some configurations, the accessory is configured to be adjustable between the plurality of interface attachment locations without being removed from the interface (optionally a strap of the interface).

In some configurations, the accessory is configured to be adjusted slidably between the plurality of interface attachment locations.

In some configurations, the accessory is attachable to the interface at a plurality of patient face locations.

In some configurations, the accessory is configured to be adjustable between the plurality of patient face locations without being removed from the interface.

In some configurations, the accessory is configured such that it may be adjusted slidably between the plurality of patient face locations.

In some configurations, the plurality of patient face locations includes proximal to the patient's cheek.

In some configurations, the plurality of patient face locations includes between the patient's eye and lip.

In some configurations, the accessory is configured to be adjustable between a plurality of attachment locations on the interface defined by at least a connection point between a strap and a main body of the nasal cannula.

In some configurations, the plurality of patient face locations corresponds with a plurality of attachment locations.

In some configurations, the accessory may be located proximal to the patient's cheek.

In some configurations, the accessory may be located between the patient's eye and lip.

In some configurations, the accessory is configured to be moveable along the length of the strap to allow a clinician to position the sensor.

In some configurations, a or the at least one securement feature is configured to retain the accessory to a strap of a nasal cannula interface.

In some configurations, the securement feature is configured to substantially prevent movement of the accessory accessary along the strap.

In some configurations, securement feature is configured to allow relative movement of the strap and accessory when a threshold force is applied.

In some configurations, the threshold force is based on the type of securement feature and the material of the strap.

In some configurations, the at least one securement feature is configured to extend from a side of the accessory opposite the sensor cavity.

In some configurations, the at least one securement feature comprises at least one arm, the arm configured to extend around at least part of a strap of a nasal cannula interface.

In some configurations, the at least one securement feature comprises a pair of arms, the arms arranged along an axis of the accessory, the axis configured to be parallel to an axis of the strap when the accessory is connected to the strap.

In some configurations the arm comprises a first portion extending from the accessory (optionally in a perpendicular direction from an outward face of the accessory), and a second portion, wherein the second portion of the arm is configured to be oriented substantially perpendicular to the associated first portion and/or parallel to the accessory.

In some configurations, the at least one securement feature comprises a pair of arms, the pair of arms extending from the accessory and towards each other.

In some configurations, each arm comprises a first portion and a second portion, the first portion extending from the accessory (optionally in a perpendicular direction from an outward face of the accessory), and the second portions of each arm configured to be oriented toward each other (optionally the second portion of each arm is oriented substantially perpendicular to the associated first portion and/or parallel to the accessory).

In some configurations, each arm comprises a first portion and a second portion, the first portion extending from the accessory (optionally in a perpendicular direction from an outward face of the accessory), and the second portions of each arm are configured to be oriented toward a center of the accessory (optionally the second portion of each is oriented substantially perpendicular to the associated first portion).

In some configurations, each arm comprises a first portion and a second portion, the first portion extending vertically upwards from the accessory and the second portion extending inwards towards a center of the accessory and/or towards the second portion of the other arm, such that the first portion and the second portion of each arm forms an angle, wherein the angle is less than 90 degrees, or about 90 degrees, or less than about 120 degrees.

In some configurations, the strap of the nasal cannula interface is configured to be connected to the accessory.

In some configurations, the securement feature is configured to receive the strap of the nasal cannula interface.

In some configurations, a gap is defined between the pair of arms.

In some configurations, a or the strap of the nasal cannula interface is configured to be insertable to the gap (optionally defined by the or a pair of arms) to be connected to the accessory.

In some configurations, the gap is defined between the pair of arms.

In some configurations, the width of the strap of the nasal cannula interface is larger than the gap.

In some configurations, the strap of the nasal cannula can only be inserted into the gap when aligned with an edge of the strap or when the strap is folded along the width of the strap.

In some configurations, the strap of the nasal cannula can only be removed from the accessory through the gap when aligned with an edge of the strap or when the strap is folded along the width of the strap.

In some configurations, the or a at least one securement feature comprises a clip.

In some configurations the clip comprises a clip arm.

In some configurations, the clip arm is connected to the accessory via a biasing element (optionally a hinged portion).

In some configurations, the clip extends from the accessory (and optionally a side of the accessory).

In some configurations, when the clip is in a closed position the clip is configured to retain the strap of the nasal cannula to the accessory.

In some configurations, when the clip is in a closed position the clip is configured to retain the strap between the clip and the nasal cannula to the accessory.

In some configurations, the clip arm comprises a contacting surface, the contacting surface configured to engage with a strap of a nasal cannula when the clip is in a or the closed position.

In some configurations, the contacting surface is located in a recess of the clip arm.

In some configurations, the recess is shaped to receive a strap of the nasal cannula.

In some configurations, the contacting surface comprises at least one protrusion, the at least one protrusion configured to aid in retaining the strap when the clip is closed.

In some configurations, the at least one protrusion comprises at least one rib, the at least one rib located perpendicular to a width of the strap when the strap is engaged with the contacting surface, and/or perpendicular to a longitudinal axis of the contacting surface.

In some configurations, the at least one rib comprises a pair of ribs spaced along a length of the strap when the strap is engaged with the contacting surface, and/or a longitudinal axis of the contacting surface.

In some configurations, the at least one rib comprises a pair of ribs located at opposing ends of the contacting surface.

In some configurations, the at least one protrusion comprises one or more bumps.

In some configurations, the one or more bumps comprises a bump located in each corner of the contacting surface.

In some configurations, the one or more bumps are patterned across at least a portion, or the entire, contacting surface.

In some configurations, the one or more bumps are patterned in offset rows or aligned rows.

In some configurations, the contacting surface has a non-smooth and/or a substantially rough surface.

In some configurations, when the clip is closed and retaining the strap of the nasal cannula, the accessory is prevented from sliding relative to the strap.

In some configurations, the clip arm comprises an aperture extending through the clip arm (optionally through the contacting surface).

In some configurations, the aperture is configured to receive a portion of another of the at least one securement system (optionally the another of the at least one securement system is at least one arm).

In some configurations, the accessory comprises a pair of arms located on each side of the clip.

In some configurations, the clip arm comprises at least one retention feature, the retention feature configured to engage with a corresponding feature of the accessory to retain the clip in a closed position.

In some configurations, a or the strap of the nasal cannula interface is configured to be connected to the accessory.

In some configurations, the securement feature is configured to receive a or the strap of the nasal cannula interface.

In some configurations, the securement feature configured to connect to a cannula connector of the nasal cannula interface, optionally, the main body connection feature being located at a first end of the accessory.

In some configurations, the cannula connector is configured to connect a main body of the nasal cannula interface to the strap of the nasal cannula interface.

In some configurations, a strap passage, the strap passage extending from a second end of the nasal cannula interface accessory along, at least a portion of, a longitudinal axis of the nasal cannula interface accessory.

In some configurations, the accessory is configured to act as a side arm (and optionally a cheek support for the interface).

In some configurations, a or the face of the accessory which is configured to face a patient in use is substantially circular.

In some configurations, a or the face of the accessory which is configured to face a patient in use comprises at least one surface material.

In some configurations, the at least one surface material covers a or the face of the accessory which is configured to face a patient.

In some configurations, the at least one surface material does not cover the sensor.

In some configurations, the at least one surface material is provided with an aperture such that the at least one surface material does not extend over the sensor cavity of the accessory.

In some configurations, the at least one surface material is the same material as the accessory.

In some configurations, the at least one surface material is different from another material of the accessory.

In some configurations, the at least one surface material and the accessory are separable.

In some configurations, the at least one surface material and the accessory are integral.

In some configurations, the at least one surface material is a film or membrane.

In some configurations, the at least one surface material is configured to increase friction between the accessory and a patient's face.

In some configurations, the surface material is configured to provide a frictional force to resist movement between the accessory and the strap.

In some configurations, the at least one surface material has adhesive properties.

In some configurations, the at least one surface material has slip-resistant material properties.

In some configurations, the at accessory has one or more gripping features.

In some configurations, a sensor is preinstalled in the sensor cavity of the accessory.

In another aspect there is provided a kit, the kit comprising a nasal cannula interface and an accessory as described herein.

In another aspect there is provided a nasal cannula interface component, the nasal cannula interface component comprising: a sensor cavity configured to retain a sensor configured to measure at least one patient parameter, a wire cavity, the wire cavity configured to provide a pathway for one or more wires to the sensor cavity, a main body connection feature, the main body connection feature configured to connect to a main body of the nasal cannula interface, the main body connection feature being located at a first end of the nasal cannula interface component, and a strap of the nasal cannula interface.

In some configurations, the component is a side arm (optionally a cheek support).

In some configurations, a strap of the nasal cannula is configured to be connected to the main body connection feature.

In some configurations, the strap and/or the main body connection feature are at least partially formed integrally (optionally overmolded) with the component.

In some configurations, a sensor is preinstalled in the sensor cavity of the accessory.

In another aspect there is provided a nasal cannula interface comprising a component as described herein.

In another aspect there is provided a component for a patient respiratory interface, the component comprising: a component body having a sensor cavity configured to retain a sensor configured to measure at least one patient parameter, a first connector, the first connector configured to connect to a body of the patient respiratory interface, and a second connector, the second connector configured to connect to a headgear of the patient respiratory interface.

In some configurations, the first connector is located at a first end of the component, and the second connector is located at a second end of the component.

In some configurations, the first connector is configured to releasably connect the component to the body of the patient respiratory interface.

In some configurations, the second connector is configured to releasably connect the component to the headgear component of the patient respiratory interface.

In some configurations, the second connector is configured to be integrally formed with the headgear component.

In some configurations, the headgear component is a strap of the patient respiratory interface.

In some configurations, the headgear component comprises at least one aperture for attachment of a strap.

In some configurations, the component is configured to be located between the body of the patient interface and the headgear component.

In some configurations, the component is configured to be located between the body of the patient interface and the headgear component when connected to the body of the patient interface and the headgear component.

In some configurations, the sensor cavity is defined by one or more walls of the component body.

In some configurations, the sensor cavity is located between the body of the patient respiratory interface and the headgear of the patient respiratory interface.

In some configurations, the component body and its sensor cavity are located between the first connector and second connector of the component.

In some configurations, the first connector, component body, and second connector are integrally formed with each other.

In some configurations, the component further comprises a wire cavity, the wire cavity configured to provide a pathway for one or more wires to the sensor cavity.

In some configurations, each of the one or more wires comprises, a cable, a cord, a lead, or any other electrically conductive material.

In some configurations, the sensor cavity is formed in a first face of the component body.

In some configurations, the wire cavity is formed in the same face, and/or a different face of the component body as is the sensor cavity.

In some configurations, the wire cavity comprises a plurality of openings on different faces of the component body (optionally adjacent faces).

In some configurations, the wire cavity is formed in the component body adjacent to the sensor cavity.

In some configurations, the sensor cavity is one or more of: square, rectangular, and/or circularly shaped.

In some configurations, the sensor cavity has substantially rounded edges and vertices.

In some configurations, the sensor comprises a light transducer (optionally an infrared transducer, or a red-light transducer) and/or a light source (optionally an infrared source, or a red light source).

In some configurations, the sensor cavity is arranged to orient the sensor such that the light transducer and/or light source faces towards the patient.

In some configurations, the sensor cavity is configured such that, when a sensor is provided within the sensor cavity, a transducer of the sensor is exposed to an outside of the component.

In some configurations, the sensor cavity comprises an opening on a face of the component body which is configured to face a patient in use.

In some configurations, the sensor cavity is configured to present the transducer of the sensor at the first face of the component body.

In some configurations, the sensor cavity is arranged to orient the sensor to be in contact with a patient.

In some configurations, the wire cavity extends from a face of the component body to the sensor cavity.

In some configurations, the wire cavity extends from an external face of the component body to the sensor cavity.

In some configurations, the wire cavity comprises an opening between a face of the component body and the sensor cavity.

In some configurations, the wire cavity is a slot that is recessed from a face of the component body.

In some configurations, the wire cavity is one or more of: substantially square, rectangular and/or circular in cross-section in at least one location along a length of the wire cavity.

In some configurations, the wire cavity is configured to face a patient in use.

In some configurations, the sensor is a patient sensor, and the patient parameter is a physiological parameter.

In some configurations, the patient parameter is a measure of blood oxygenation of the patient.

In some configurations, a patient contacting face of the component body which is configured to face a patient in use is substantially circular.

In some configurations, a or the face of the component body which is configured to face a patient in use comprises at least one surface material.

In some configurations, the at least one surface material covers a or the face of the component body which is configured to face a patient.

In some configurations, the at least one surface material does not cover the sensor.

In some configurations, at least one surface material is provided with an aperture such that the at least one surface material does not extend over the sensor cavity of the component.

In some configurations, the at least one surface material is the same material as the component.

In some configurations, the at least one surface material is different from another material of the component.

In some configurations, the at least one surface material and the component are separable.

In some configurations, the at least one surface material and the component are integral.

In some configurations, the at least one surface material is a film or membrane.

In some configurations, the at least one surface material is configured to increase friction between the component and a patient's face.

In some configurations, the at least one surface material has adhesive properties.

In some configurations, the at least one surface material has slip-resistant material properties.

In some configurations, the component has one or more gripping features.

In some configurations, a sensor is preinstalled in the sensor cavity of the component.

In some configurations, when the first connector is connected to a body of the patient interface and the second connector is connected to part of the headgear or system for securing the patient interface to a patient, the sensor cavity is held against a patient's skin surface.

In some configurations, the patient respiratory interface is one of: a nasal cannula interface, a nasal pillow interface, a nasal mask interface, a full-face mask interface, and an oral interface.

In another aspect there is provided a patient interface, the patient interface comprising: a headgear or system for securing the patient interface in place about a patient's head, and a component.

In some configurations, the component comprises a component body having a sensor cavity configured to retain a sensor configured to measure at least one patient parameter, a first connector, the first connector configured to connect to a body of the patient respiratory interface, and a second connector, the second connector configured to connect to a headgear of the patient respiratory interface.

The component may comprise any of the features and/or functions described herein, for example as described in any one or more of paragraphs [0121] to [0171].

In some configurations, the headgear in the form of at least one strap.

In some configurations, the strap in-use, is splittable or bifurcatable to provide an upper strap part and a lower strap part of said headgear.

In some configurations, the patient interface is a nasal cannula.

In some configurations, the patient interface is a nasal cannula and comprises one or a pair of nasal prongs.

In some configurations, the nasal cannula comprises a body, and wherein the nasal prong or prongs are integrally moulded with or removably attached to the body.

In some configurations, the body comprises a pair of side arms extending from either side of body.

In some configurations, the patient interface comprises a sensor located in the sensor cavity of the patient respiratory interface component.

A patient respiratory interface comprising:
a first side arm, the first side arm comprising a sensor recess,
a gases inlet conduit configured to convey a flow of breathable gases to a patient through a main body of the patient respiratory interface, and
a component having a conduit retaining portion configured to retain the gases inlet conduit and a sensor mount configured to retain a patient sensor,
wherein at least a portion of the sensor mount is insertable into the sensor recess of the first side arm such that when the patient respiratory interface is in use the patient sensor contacts the patient's face.

A first end of the gases inlet conduit connects to the main body of the patient respiratory interface.

The patient respiratory interface further comprises a second side arm, the second side arm comprising a sensor recess configured to retain a patient sensor.

At least a portion of the sensor mount of the component is insertable into the sensor recess of the second side arm.

The first end of the gases inlet conduit is connectable to the main body in a plurality of different directions.

The first end of the gases inlet conduit is connectable to the main body at each of two laterally opposed sides of the main body, being a first side adjacent the first side arm and a second side adjacent the second side arm.

When the gases inlet conduit is connected to the main body at its first side, the sensor mount is insertable into the sensor recess of the first side arm.

When the gases inlet conduit is connected to the main body at its second side, the sensor mount is insertable into the sensor recess of the second side arm such that when the patient respiratory interface is in use the patient sensor contacts the patient's face.

When the gases inlet conduit is connected to the main body at its first side and the sensor mount is inserted into the sensor recess of the first side arm, a curvature of the gases inlet conduit between its first end and the conduit retaining portion of the component is less than about 90 degrees.

When the gases inlet conduit is connected to the main body at its second side and the sensor mount is inserted into the sensor recess of the second side arm, a curvature of the gases inlet conduit between its first end and the conduit retaining portion of the component is less than about 90 degrees.

The sensor recess of the first side arm and sensor mount of the component are configured, when the sensor mount is inserted into the sensor recess of the first side arm, to become engaged with each other such as to resist withdrawal of the sensor mount from the sensor recess.

The sensor recess of the second side arm and sensor mount of the component are configured, when the sensor mount is inserted into the sensor recess of the second side arm, to become engaged with each other such as to resist withdrawal of the sensor mount from the sensor recess.

The gases inlet conduit comprises one or more electrically conductive elements extending along at least a portion of its length.

The gases inlet conduit has a second end opposite the first end, and the second end is for connection to an inspiratory conduit to receive a respiratory gases flow therefrom and the second end of the gases inlet conduit comprises one or more electrical connectors to interface with a corresponding one or more electrical connectors of the inspiratory conduit when connected thereto.

The one or more electrically conductive elements extend from the second end of the gases inlet conduit to a location on along the gases inlet conduit at which the conduit retaining portion of the component is to retain the gases inlet conduit.

The one or more electrically conductive elements include one or more sensor wires, when the conduit retaining portion of the component retains the gases inlet conduit the one or more sensor wires are in electrical communication with the patient sensor.

The gases inlet conduit comprises a first portion and a second portion, and the conduit retaining portion of the component interfaces between the first portion and second portion of the gases inlet conduit.

The gases inlet conduit comprises a single unitary conduit between its first end and second end.

The conduit retaining portion of the component is permanently attached to the gases inlet conduit.

The conduit retaining portion of the component is removably attachable to the gases inlet conduit.

The gases inlet conduit is slidable along its length relative to the component when retained by the conduit retaining portion.

The gases inlet conduit is not slidable along its length relative to the component when retained by the conduit retaining portion.

The conduit retaining portion retains the gases inlet conduit about a periphery of the gases inlet conduit.

The conduit retaining portion retains the gases inlet conduit about more than half of a perimeter of the gases inlet conduit.

The conduit retaining portion encircles the gases inlet conduit.

The clip attaches about more than half of a perimeter of the gases inlet conduit.

The clip encircles the gases inlet conduit.

The or each sensor recess comprises an opening through the side arm between a non-patient-facing side and a patient-facing side of the side arm.

Only a portion of the patient sensor is within the sensor recess when the sensor mount is inserted and retained within the sensor recess.

The or each sensor recess is square, rectangular, or circular in cross-section.

A cross-section of the sensor mount or at least a portion thereof substantially corresponds to the cross-sectional shape of the sensor recess.

The patient sensor comprises a light transducer (optionally an infrared transducer, or a red light transducer) and/or a light source (optionally an infrared source, or a red light source).

The or each side arm is/are configured such that the or each sensor recess is located, when the patient respiratory interface is in use, at a cheek of the patient.

The or each side arm is/are configured such that the or each sensor recess is located, when the patient respiratory interface is in use, between an eye of the patient and the patient's lip.

The portion of the sensor mount which is insertable into the or each sensor recess includes, when the patient sensor is retained within the sensor mount, a transducer of the patient sensor.

When retained within the or each sensor recess, the transducer of the patient sensor is configured to contact face of a patient when the respiratory patient interface is worn.

A nasal cannula interface and an accessory for the same, the accessory comprising:
a sensor cavity, the sensor cavity configured to retain a sensor configured to measure at least one patient parameter, and
at least one securement feature, the at least one securement feature configured to connect the accessory to a nasal cannula interface (optionally to a strap of a nasal cannula interface), and
the nasal cannula comprising a first prong and a second prong, the prongs configured to cause an asymmetrical flow of gases at a patient's nares.

The first prong has a first shape and the second prong has a second shape.

The first prong has a smaller inner cross-sectional area in a direction transverse to gases flow through the first prong than a corresponding inner cross-sectional area of the second prong in a direction transverse to gases flow through the second prong.

The first and second prongs are configured such that at least about 60% of a total volumetric flow rate of gases flow dispensed by the nasal cannula is delivered out of the second prong.

A nasal cannula interface and component for the same, the nasal cannula interface component comprising:
a sensor cavity configured to retain a sensor configured to measure at least one patient parameter,
a wire cavity, the wire cavity configured to provide a pathway for one or more wires to the sensor cavity, and
a main body connection feature, the main body connection feature configured to connect to a main body of the nasal cannula interface, the main body connection feature being located at a first end of the nasal cannula interface component, and
the nasal cannula interface comprising:
a first prong and a second prong, the prongs configured to cause an asymmetrical flow of gases at a patients nares, and
a strap.

The first prong has a first shape and the second prong has a second shape.

The first prong has a smaller inner cross-sectional area in a direction transverse to gases flow through the first prong than a corresponding inner cross-sectional area of the second prong in a direction transverse to gases flow through the second prong.

The first and second prongs are configured such that at least about 60% of a total volumetric flow rate of gases flow dispensed by the nasal cannula is delivered out of the second prong.

A patient respiratory interface comprising:
a first prong and a second prong, the prongs configured to cause an asymmetrical flow of gases at a patients nares,
a first side arm, the first side arm comprising a sensor recess,
a gases inlet conduit configured to convey a flow of breathable gases to a patient through a main body and the first and second prongs of the patient respiratory interface,
a clip attachable to the gases inlet conduit, the clip configured to retain a patient sensor within a sensor mount of the clip,
wherein at least a portion of the sensor mount is insertable into the sensor recess of the first side arm such that when the patient respiratory interface is in use the patient sensor contacts the patient's face.

The first prong has a first shape and the second prong has a second shape.

The first prong has a smaller inner cross-sectional area in a direction transverse to gases flow through the first prong than a corresponding inner cross-sectional area of the second prong in a direction transverse to gases flow through the second prong.

The first and second prongs are configured such that at least about 60% of a total volumetric flow rate of gases flow dispensed by the nasal cannula is delivered out of the second prong.

A nasal cannula interface and an accessory for the same, the accessory comprising:
a sensor cavity, the sensor cavity configured to retain a sensor configured to measure at least one patient parameter, and
at least one securement feature, the at least one securement feature configured to connect the accessory to a nasal cannula interface (optionally to a strap of a nasal cannula interface), and
the nasal cannula comprising a first prong and a second prong, and a cross-sectional area of the first prong is smaller than a cross-sectional area of the second prong.

The cross-sectional area of the first prong which is smaller than the cross-sectional area of the second prong are taken at corresponding locations on the first and second prongs.

The cross-sectional area of the first prong and second prongs is taken at respective proximal openings of the prongs.

The difference in cross-sectional area of the first prong and the second prong is such as to cause an asymmetrical flow of gases at a patient's nares.

A patient respiratory interface and an accessory for the same, the accessory configured to retain a patient sensor to measure a patient parameter, wherein the accessory is configured to interact with the patient respiratory interface and measure a physiological parameter of the patient when the patient respiratory is worn by a patient.

The interaction of the accessory with the patient respiratory interface comprises an attachment of the accessory to the patient respiratory interface.

The interaction of the accessory with the patient respiratory interface comprises an attachment of the accessory to a strap of the patient respiratory interface.

The accessory is substitutable for a first component of the patient respiratory interface, and the interaction of the accessory with the patient respiratory interface comprises the substitution of the accessory for the first component of the nasal canula interface.

The interaction of the accessory with the patient respiratory interface further comprises the interconnection of the accessory with a second component of the patient respiratory interface.

The interaction of the accessory with the patient respiratory interface further comprises the interconnection of the accessory with both the second and a third component of the patient respiratory interface.

The first component of the patient respiratory interface is a side arm of the patient respiratory interface.

The first component of the patient respiratory interface is a connector, for connecting together two other components of the patient respiratory interface.

The first component of the patient respiratory interface is a connector to connect together a main body of the patient respiratory interface and a headgear of the patient respiratory interface.

The interaction of the accessory with the patient respiratory interface is such that a patient sensor retained by the accessory, when associated with the patient respiratory interface and the patient respiratory interface is worn by a patient, is located against the patient's face between a mouth region and an eye region of the patient's face.

The patient respiratory interface is a nasal cannula interface.

A patient respiratory interface for delivering a respiratory therapy to a patient, the patient respiratory interface comprising:
a gases inlet conduit configured to convey a flow of breathable gases to a patient through a main body of the patient respiratory interface,
a patient sensor having a sensor mount,
a retaining structure connectable at each of a first and second laterally opposed sides of the main body to pass around a patient's head and retain the main body thereon, the retaining structure comprising:
a first component located along the retaining structure laterally of where the retaining structure is connectable with the first side of the main body and configured to receive the sensor mount such that when the patient respiratory interface is in use the patient sensor contacts the patient's face.

The patient respiratory interface further comprises a second component located along the retaining structure laterally of where the retaining structure is connectable to the second side of the main body and configured to receive the sensor mount such that when the patient respiratory interface is in use the patient sensor contacts the patient's face.

When the sensor mount is received by the first component and the patient respiratory interface is in use the patient sensor contacts a first side of the patient's face.

When the sensor mount is received by the second component and the patient respiratory interface is in use the patient sensor contacts a second side of the patient's face, being a side opposite the first side.

The first component has a sensor cavity within which the sensor mount is able to be inserted and retained.

The first component has a sensor recess which extends through the first component from a non-patient-facing side to a patient-facing side of the first component, and the sensor mount is able to be inserted and retained within the sensor recess.

The first component is a first side arm.

The first component is or includes a strap connector for a strap of the retaining structure.

The first component is or includes a connector between a strap of the retaining structure and a side arm of the patient respiratory interface.

The patient sensor is partially overmolded by the first component.

The second component has a sensor recess which extends through the second component from a non-patient-facing side to a patient-facing side of the second component, and the sensor mount is able to be inserted and retained within the sensor recess.

The second component is a second side arm.

The second component is or includes a strap connector for a strap of the retaining structure.

The second component is or includes a connector between a strap of the retaining structure and a side arm of the patient respiratory interface.

The patient sensor is a first patient sensor and the patient respiratory device further comprises a second patient sensor, and the second patient sensor is partially overmolded by the first component.

When inserted and retained within the sensor cavity of the first component and the patient respiratory interface is in use, the patient sensor contacts a first side of the patient's face and when inserted and retained within the sensor cavity of the second component the patient sensor contacts a second side of the patient's face.

When inserted and retained within the sensor recess of the first component and the patient respiratory interface is in use, the patient sensor contacts a first side of the patient's face and when inserted and retained within the sensor recess of the second component the patient sensor contacts a second side of the patient's face.

An accessory for a patient respiratory interface, the accessory comprising a sensor cavity and being configured to retain a sensor therein to measure a patient parameter, wherein the accessory is interconnectable with one or more components of the patient respiratory interface and is configured, when interconnected with the patient respiratory system and the patient respiratory interface worn by a patient, to present the patient sensor at a face surface of the patient.

The accessory is configured to interconnect with one component of the patient respiratory interface.

The accessory is configured to interconnect with a side arm of the patient respiratory interface, and more particularly with a sensor recess of a side arm of the patient respiratory interface.

The accessory is substitutable for a first component of the patient respiratory interface.

The first component of the patient respiratory interface is a side arm of the patient respiratory interface.

The first component of the patient respiratory interface is a connector, for connecting together two other components of the patient respiratory interface.

The accessory is configured to interconnect between a second and third components of the patient respiratory interface.

The second component is a main body of the patient respiratory interface, and the third component is a headgear connector of the patient respiratory interface.

The second component is a side arm of the patient respiratory interface, and the third component is a headgear strap of the patient respiratory interface.

The patient respiratory interface is a nasal cannula interface.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 shows schematically a respiratory apparatus configured to provide a respiratory therapy to a patient.

FIG. 2 shows a schematic diagram of a closed loop control system for use with the respiratory apparatus of FIG. 1.

FIG. 3 shows a nasal cannula interface in use with a patient in accordance with an aspect of this disclosure.

FIG. 4 shows a partial front view of the nasal cannula interface in accordance with an aspect of this disclosure.

FIG. 5 shows an exploded view of the nasal cannula interface of FIG. 4.

FIG. 6A shows a front view of a body of the nasal cannula interface in accordance with an aspect of this disclosure.

FIG. 6B shows a patient respiratory interface having a nasal interface or more particularly nasal cannula interface with asymmetric delivery elements.

FIGS. 7A and 7B respectively show perspective and perspective exploded views of another nasal cannula interface in accordance with this disclosure, whilst FIG. 7C shows a perspective view of headgear of the nasal cannula interface.

FIGS. 7D to 7F show enlarged perspective views of a headgear connector connecting to a nasal cannula interface in accordance with this disclosure.

FIGS. 7G and 7H respectively show cross-sectional views corresponding to FIGS. 7D and 7E.

FIGS. 8A and 8B show enlarged perspective views of the connection between the nasal cannula interface and the headgear, showing forms of a side arm of the nasal cannula interface.

FIGS. 9A to 9C show enlarged perspective views of a retention clip of the nasal cannula interface in accordance with this disclosure.

FIGS. 10A to 11B show views of an accessory without a sensor for use with a nasal cannula interface.

FIGS. 12A to 13D show views of an accessory with a sensor for use with a nasal cannula interface.

FIGS. 14A and 14B show views of an accessory without a sensor for use with a nasal cannula interface.

FIGS. 15A and 15B show views of an accessory without a sensor for use with a nasal cannula interface.

FIGS. 15C and 15D show views of an accessory with a sensor for use with a nasal cannula interface.

FIGS. 15E and 15F show views of an accessory connected to a nasal cannula interface.

FIGS. 16A and 16E show views of an accessory without a sensor for use with a nasal cannula interface.

FIGS. 17A and 17B show views of an accessory without a sensor for use with a nasal cannula interface.

FIG. 17C shows a cross section of the accessory of FIGS. 17A and 17B.

FIGS. 18A to 18C show views of an accessory with a sensor for use with a nasal cannula interface.

FIG. 19A is a view of a patient respiratory interface that includes a component that retains a sensor.

FIG. 19B is a different perspective view of the portion A of FIG. 19A.

FIG. 19C is a perspective view taken from the opposite side of the component as illustrated in FIG. 19B.

FIG. 20 is a view of a component of a patient respiratory interface that can retain a sensor.

FIG. 21 is a view of the component of FIG. 20 with a sensor retained by the component.

FIG. 22 is an exploded view of a component of or for a patient respiratory interface.

FIG. 23 is a perspective view of a connector.

FIG. 24 is a cross sectional view of the connector of FIG. 23, before the clip is engaged with the detent.

FIG. 25 is a cross sectional view of the connector of FIG. 23, showing the initial stages of the clip being engaged with the detent.

FIG. 26 is a cross sectional view of the connector of FIG. 23 showing the slide in the secured position.

FIG. 27 is a cross sectional view of the connector of FIG. 23 showing the slide in a free position.

FIG. 28 is a cross sectional view of the connector of FIG. 23 showing the clip removed from the carrier.

FIG. 29 is a cross sectional view of one half of the slide of the connector of FIG. 23.

FIG. 30 is a cross sectional view of the other half of the slide of the connector of FIG. 23.

FIG. 31 is a perspective view of the carrier of the connector of FIG. 23.

FIG. 32 is a perspective view of the clip of the connector of FIG. 23.

FIG. 33 is a perspective view of a conduit and wire clip.

FIG. 34 is an end view of the conduit and wire clip of FIG. 33.

FIG. 35 is a view of part of a conduit and wire attached together using a conduit and wire clip.

FIG. 36A is a view of another nasal cannula interface that includes a component configured to retain a patient sensor.

FIG. 36B is a view of the component of FIG. 36A that is configured to retain a sensor.

FIG. 37 is a view of the nasal cannula interface of FIG. 36A, configured so the conduit is connectible at either side of the main body of the interface.

FIG. 38A is a view of another nasal cannula interface that includes a component able to retain a patient sensor.

FIG. 38B is a view of the component of FIG. 38A that is configured to retain a sensor.

FIG. 39 is a view of a headgear strap and connectors for a patient respiratory interface, where one of the connectors is a component able to retain a patient sensor.

FIG. 40 is a partial and partially exploded view of a nasal cannula interface having a component able to retain a patient sensor where the component is provided as an intermediate connector between a side arm and a headgear connector of the nasal cannula interface.

FIG. 41 is a view of communication modules for a component according to the disclosure that is configured to retain a patient sensor.

FIG. 42 is a cross-sectional view of a component which is configured to retain a patient sensor.

FIG. 43 is a partial and partially exploded view of a nasal cannula interface having a component which is configured to retain a patient sensor.

FIGS. 44A-C are, respectively, a top view, a front view, and a bottom view of a nasal cannula interface having asymmetric prongs.

### DETAILED DESCRIPTION

Certain embodiments and examples of a respiratory apparatus for providing respiratory support to a patient, and patient interfaces for such a system, are described herein. Those of skill in the art will appreciate that the disclosure extends beyond the specifically disclosed embodiments and/or uses and obvious modifications and equivalents thereof. Thus, it is intended that the scope of the disclosure herein disclosed should not be limited by any particular embodiments described herein.

Patients suffering from various health conditions and diseases can benefit from respiratory support. For example, patients suffering from conditions such as chronic obstructive pulmonary disease (COPD), pneumonia, asthma, bronchopulmonary dysplasia, heart failure, cystic fibrosis, sleep apnea, lung disease, trauma to the respiratory system, acute respiratory distress, receiving pre- and post-operative oxygen delivery, and other conditions or diseases can benefit from respiratory support. As a part of providing a patient with respiratory support, one or more physiological parameters of the patient may be measured by a patient sensor for the purpose of monitoring the patient's health. The patient sensor may be a pulse oximeter, which provides information relating to heart rate and blood oxygen saturation (SpO₂).

When providing a patient with respiratory support, in particular supplemental oxygen therapy, a common method of monitoring the patient's health is to ensure that their SpO₂ does not drop too low (e.g., typically below about 90%). However, supplying the patient with too much oxygen can over oxygenate their blood, and is also considered dangerous. Generally, the patient's SpO₂ is kept in a range from about 80% to about 99%, and preferably about 92% to about 96%, although these ranges may differ due to patient conditions, and/or from patient to patient.

Due to various patient factors such as respiratory rate, lung tidal volume, heart rate, activity levels, height, weight, age, gender, and other factors, there is no one prescribed level of supplemental oxygen that can consistently achieve an SpO₂ response in the targeted range for each patient. Individual patients regularly need their fraction of oxygen delivered to the patient (FdO₂) monitored and adjusted to ensure they are receiving the correct FdO₂ to achieve the targeted SpO₂. Achieving a correct and consistent SpO₂ is an important factor in treating patients with various health conditions or diseases. Additionally, patients suffering from these health problems may find benefit from a system that automatically controls oxygen saturation. The present disclosure is applicable to a wide range of patients that require fast and accurate oxygen saturation control.

The fraction of oxygen delivered to a patient (FdO₂) may be controlled manually. For example, a user can manually adjust an oxygen supply valve to change the flow rate or fraction of oxygen being delivered to the patient. The user can determine SpO₂ levels of the patient using a patient monitor, such as a pulse oximeter. The SpO₂ measurements can be displayed on a respiratory apparatus 10 or on the pulse oximeter itself. The user can continue to manually adjust the amount of oxygen being delivered to the patient until the SpO₂ level of the patient reaches a determined level. The process of monitoring SpO₂ levels, and adjusting the amount of oxygen delivered accordingly, may be performed in various settings. For example: hospital, palliative care, hospice, or a domestic setting (for example a patient's home).

A patient interface (elsewhere ‘patient respiratory interface’) for a respiratory apparatus may have a main body sized and shaped to deliver respiratory support through the patient's airway. Patient interfaces are available in a range of styles including full-face, nasal, direct nasal, and oral masks, which create a substantially airtight seal with the nose and/or mouth. A patient interface may be an indirect interface which covers the nose, mouth, or both, or an indirect interface such as an interface comprising nasal nozzles or pillows or similar which enter the nares of the wearer. For example, a patient interface may have a main body in the form of a nasal cannula.

A patient respiratory interface that includes or is for use with an accessory or component as described herein may comprise nasal interfaces that can be used to deliver a high flow of gases to a patient. Nasal delivery elements, such as nasal prongs which may optionally comprise nasal pillows, are inserted into the nose of a patient to deliver the required therapy. The nasal delivery elements may be desired to seal or partially occlude at the nose, or may not be required to seal at the nose, to deliver the therapy.

Nasal prongs typically refer to nasal delivery elements designed to not seal or to only partially occlude at the nose. When one or more of the nasal prongs comprises a nasal pillow, the nasal delivery elements are designed to seal at the nose. Nasal high flow (NHF) typically is a non-sealing therapy that delivers relatively high-volume flow to the patient through a patient interface, such as a nasal interface. A nasal interface as herein described may refer to, but is not limited to, a nasal cannula.

An asymmetrical interface or asymmetrical nasal delivery elements, as described herein, refers to an interface where the nasal delivery elements differ in size such as internal and/or external transverse dimensions or diameters, and/or internal and/or external cross-sectional areas. The external cross-sectional area is the cross-sectional area bounded by the outer wall of the nasal delivery element. For non-circular cross-sections, the references herein to a diameter may be interpreted as a transverse dimension. In some configurations, references herein to a diameter include but are not limited to a hydraulic diameter.

An asymmetrical interface allows an asymmetrical flow to be delivered through the interface to both nares or to either naris. Asymmetrical flow as described herein refers to a flow that differs within the interface or within the nose or within the interface and the nose. In this way, a different flow may be delivered by each nasal delivery element, or the flow may differ between inspiration and expiration, or the delivered flow may be a combination of the above. An asymmetrical flow may also include partial unidirectional flow.

Delivery of asymmetrical flow may improve clearance of dead space in the upper airways, decrease peak expiratory pressure, increase safety of the therapy particularly for children and infants, and reduce resistance to flow in the interface. An asymmetrical nasal interface and/or nasal delivery elements as described herein includes patient interfaces configured to produce such asymmetrical flow through asymmetrical nasal delivery elements.

Pressure generated by NHF depends on flow through the nasal interface, the size of the nasal delivery elements and/or nares of the patient, and the breathing cycle. If flow, leak, or a combination of flow and leak, is asymmetrical through the nasal interface, the flow through the nose may become asymmetrical during breathing. Partial and total unidirectional flow may be types of asymmetrical flow. Partial or total unidirectional flow may provide improved clearance of anatomical dead space as the air is continuously flushed from the upper airways. Partial unidirectional flow may be more comfortable than total unidirectional flow. Total unidirectional flow as described herein includes flow entering one naris by a nasal delivery element and exiting via the other naris via a nasal delivery element, venting to the atmosphere, due to the absence of a nasal delivery element, or the like. Partial unidirectional flow as described herein includes flow that may enter the nose via both nares and leave the nose from one naris, flow that may enter the nose through one naris and leave the nose via both nares, or different proportions of flow that may enter the nose through both nares and different proportions of flow that may leave the nose through both nares, and may be flow that may enter the nose via both nares and leave the nose from one or both nares and optionally via the mouth.

NHF delivered through an asymmetrical nasal interface can involve making an interface in which the nasal delivery elements are of different size, e.g. different length and/or internal diameter or cross-sectional area and/or external diameter or cross-sectional area. Particularly for children or infants, nasal delivery elements will have a small internal diameter and thus higher resistance to gas flow. By using nasal delivery elements that are different lengths, each nasal delivery element may have a different internal diameter (e.g., minimum internal diameter or area). A longer nasal delivery element may have a smaller internal diameter and higher resistance to gas flow; a shorter nasal delivery element may have a larger internal diameter (e.g., larger minimum internal diameter), hence lower resistance to gas flow at the interface. A decreased resistance to flow allows the desired flow to be achieved using lower backpressure, or a lower motor speed of the gas generating device, or a combination of the two.

Asymmetrical nasal delivery elements may cause the peak expiratory pressure to decrease due to the different cross-sectional areas of the nasal delivery elements at the nose which may provide different internal diameters for each nasal delivery element.

The pressure when exhaling against the asymmetric nasal interface may be higher than with a symmetric one, which is beneficial as higher positive-end expiratory pressure (PEEP) is part of the treatment for COPD (pressure here referring to the intrathoracic pressure). Expiratory pressure is dependent on the combined cross-sectional area of the two prongs. Increasing the cross-section of symmetric prongs carries the risk of fully occluding the patient's nares. Using asymmetric prongs allows for an increase in total cross-sectional area without the accompanying occlusion risk. The partially unidirectional flow may reduce turbulence in the patient's nasal cavity, which could improve comfort.

A patient interface may include a headgear (or a ‘head securement assembly’) to retain the patient interface on the patient's face. A headgear or head securement assembly may include one or more straps.

In some arrangements, a patient interface may include one or more components that interconnect between the main body and headgear of the patient interface. For example, one or more arms and/or one or more buckles or connectors may connect together the main body of the patient interface and the headgear.

When a patient sensor is used as a part of the respiratory apparatus, the user is required to mount the patient sensor to the patient. This adds another task required of the user to an already potentially large set of tasks required for setting up the respiratory apparatus. Additionally, the separate patient sensor can lead to problems such as incorrect mounting of the sensor leading to incorrect measurement and/or the sensor falling off during use.

The user of the apparatus may be for example a nurse, a doctor, any other medical clinician, or the patient themselves.

As such, a patient interface which incorporates the patient sensor allows the use of the patient sensor without increasing the workload of the user. In particular, it may simplify or remove the task of mounting the sensor. This may have benefits in a hospital setting, in which a single clinician might have a large group of patients that they need to address. Additionally, this may have benefits in a home setting, as it simplifies the setup process for a patient who may need to perform these tasks themselves. Furthermore, integrating the patient sensor into the patient interface helps ensure correct orientation of the patient sensor and prevents the patient sensor from falling off during use.

In some cases, for example when a patient changes to a new therapy, and/or requires different respiratory support, a patient sensor may need to be added to the system, and it may be that a new patient interface able to accommodate the sensor may be required.

A patient sensor may be used with a patient interface by way of an accessory. The accessory is configured to retain a patient sensor for measuring at least one patient parameter. The accessory is configured to attach to the patient interface.

For example, such an accessory may be configured to attach to a strap or arm of a patient interface to position the sensor against the patient's face. The accessory may also correctly position the patient sensor relative to the patient's face and the patient interface to ensure that a repeatable sensor output is obtained.

The use of the accessory also allows for the patient sensor to be removed when the patient changes to a different respiratory support (for example when the patient is deescalated) and/or changes to a different therapy by simply removing the accessory, without having the replace the entire patient interface.

The accessory may be provided separately to the patient interface or as part of a kit including the patient interface.

In some configurations, the accessory may be connectable to a headgear portion such as a strap of a different device than the patient interface which is being used by the patient.

The accessory may be described as a component of the patient interface particularly where the accessory is substitutable for a conventional component of the patient interface. In such configurations, an accessory may be referred to simply as a component of or for a patient interface.

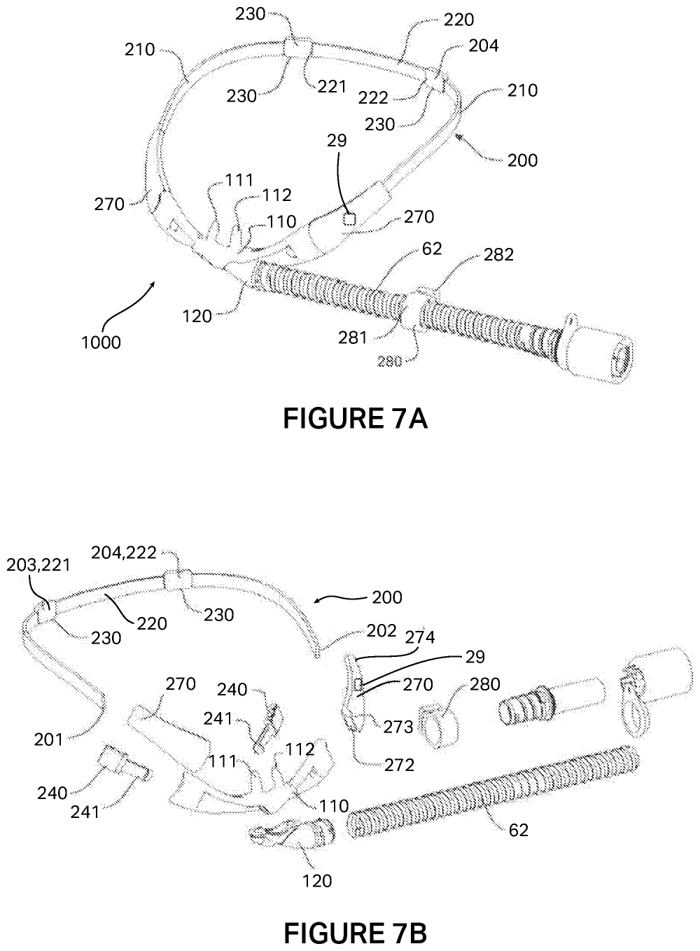

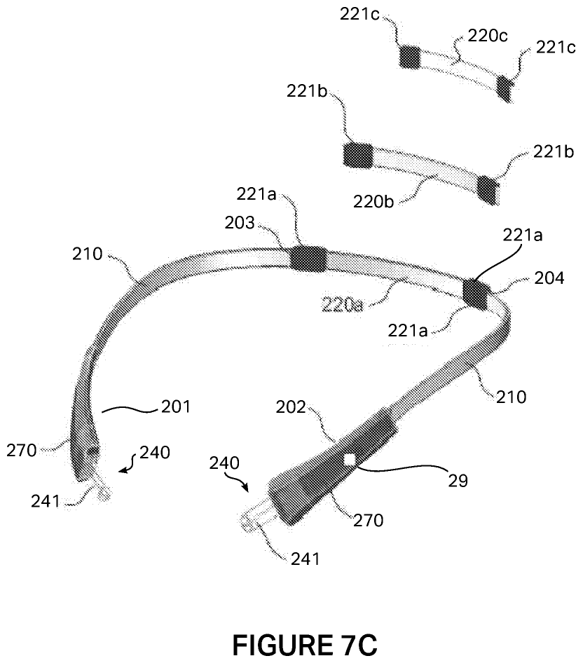

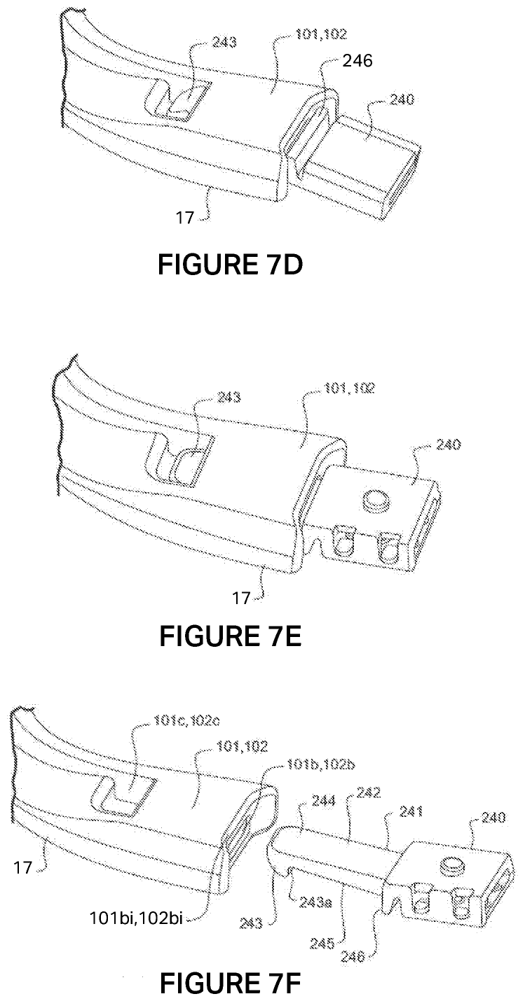

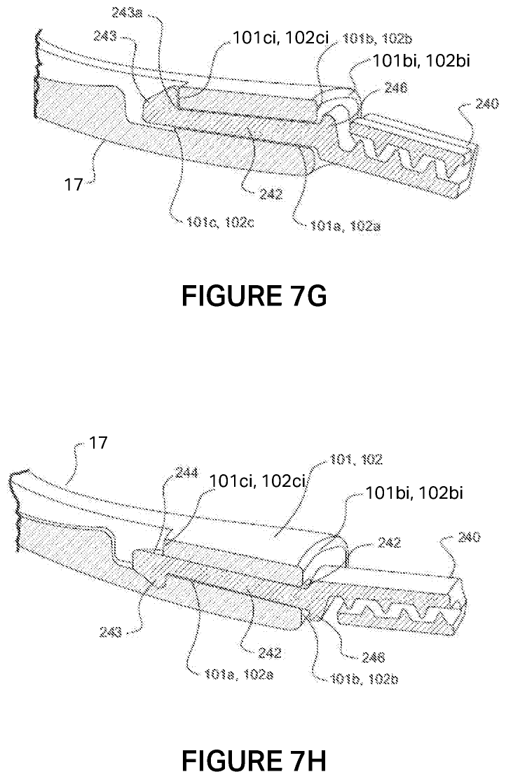

For example, where an accessory includes buckle and/or connector features, the accessory may be substituted as a component of the patient interface to replace a conventional buckle or connector between two other parts of the patient interface, such as between the main body and a strap of the headgear. As a further example, as subsequently described in relation to FIGS. 7A to 9C, the accessory may function as a component of the patient interface and more particularly as a side arm of the patient interface. The accessory of FIGS. 7A to 9C may replace a conventional side arm of a patient interface.

An accessory which can be substituted for another component of the patient interface may be provided in the place of that component when the patient interface is supplied to the patient. In other arrangements, an accessory which can be substituted for another component of the patient interface may be provided in a kit that includes the patient interface, so that a user may substitute the accessory into the patient interface. In still other arrangements, an accessory which can be substituted for another component of the patient interface may be provided separately to the patient interface.

Where an accessory is substitutable for a conventional component of the patient interface the component which the accessory defines may interconnect with one or more other components of the patient interface.

This disclosure references conduit heaters, which is a broad term and is to be given its ordinary and customary meaning to a person of ordinary skill in the art (that is, it is not to be limited to a special or customized meaning) and includes, without limitation, one or more heater strips, one or more heater wires, and/or one or more conductive elements that produce heat when electrical power is provided. Examples of such conduit heaters include wires made of a conductive metal (e.g., copper), conductive polymers, conductive inks printed on a surface of a conduit, conductive materials used to create a track on a conduit, and the like.

Furthermore, the disclosure references conduits, limbs, and medical conduits in the context of gas delivery. Conduit, for example, is a broad term and is to be given its ordinary and customary meaning to a person of ordinary skill in the art and includes, without limitation, passageways having a variety of cross-sections such as cylindrical and non-cylindrical passageways.

The disclosed systems, apparatuses and medical conduits can also be used in breathing circuits configured to provide a continuous, variable, or bi-level positive airway pressure (PAP) therapy or other form of respiratory support such as high flow or low flow oxygen therapy. The breathing circuit may for example comprising an inspiratory circuit which at a minimum includes the inspiratory gases pathway (including all the components) from the gases supply to the patient interface.

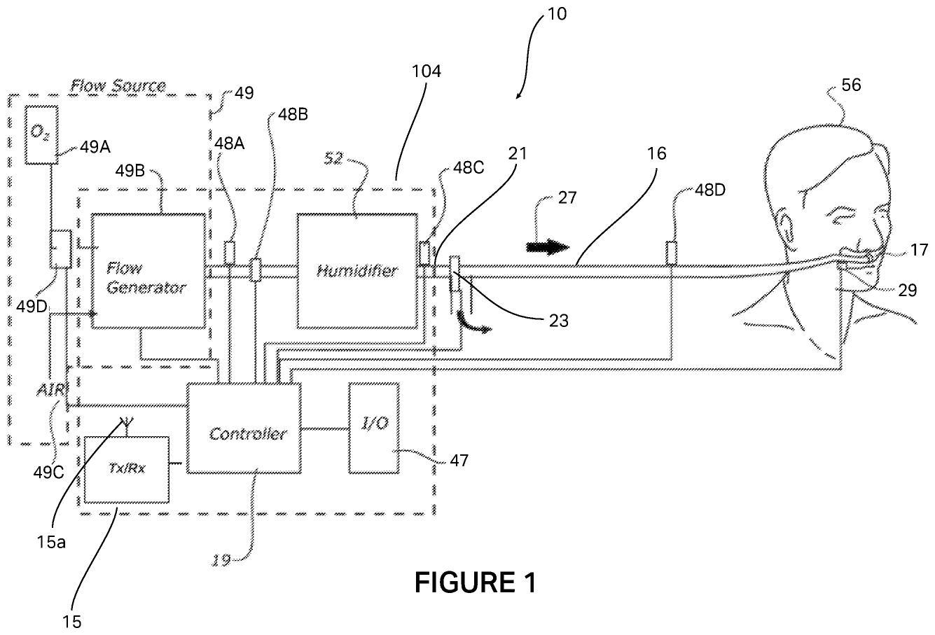

A schematic representation of the example respiratory apparatus 10 is provided in FIG. 1.

The respiratory apparatus 10 comprises a flow source 49 for providing a high flow gas 27 such as air, oxygen, air blended with oxygen, or a mix of air and/or oxygen and one or more other gases. Alternatively, the respiratory apparatus can have a connection for coupling to a flow source. As such, the flow source might be considered to form part of the apparatus or be separate to it, depending on context, or even part of the flow source forms part of the apparatus, and part of the flow source falls outside of the apparatus. In short, depending on the configuration (some components may be optional), the system can include a combination of components selected from the following:
a flow source,
humidifier for humidifying the gas-flow,
conduit (e.g., dry line or heated breathing tube),
patient interface,
non-return valve,
filter.

The conduit may comprise an inspiratory conduit 16, an expiratory conduit, or a gases inlet conduit 62.

The apparatus will be described in more detail.

The flow source could be an in-wall supply of oxygen, a tank of oxygen 49A, a tank of other gas and/or a high flow apparatus with a flow generator 49B. FIG. 1 shows a flow source 49 with a flow generator 49B, with an optional air inlet 49C and optional connection to an oxygen (O₂) source (such as tank or O₂ generator) 49A via a shut off valve and/or regulator and/or other gas flow control 49D, but this is just one option. The flow generator 49B can control flows delivered to the patient 56 using one or more valve, or optionally the flow generator 49B can comprise a blower. The flow source could be one or a combination of a flow generator 49B, O₂ source 49A, air source 49C as described. The flow source 49 is shown as part of the apparatus 10, although in the case of an external oxygen tank or in-wall source, it may be considered a separate component, in which case the apparatus has a connection port to connect to such flow source. The flow source provides a (preferably high) flow of gas that can be delivered to a patient via a inspiratory conduit 16, and patient interface 17.

The patient interface 17 may be an unsealed (non-sealing) interface (for example, when used in high flow therapy) such as a non-sealing nasal cannula, or a sealed (sealing) interface (for example, when used in CPAP therapy) such as a nasal mask, full face mask, or nasal pillows. In some embodiments, the patient interface 17 is a non-sealing patient interface which would, for example, help to prevent barotrauma (e.g., tissue damage to the lungs or other organs of the patient's respiratory system due to a difference in pressure relative to the atmosphere). In some embodiments, the patient interface 17 is a sealing mask that seals with the patient's nose and/or mouth. The patient interface may be a nasal cannula with a gases flow part (for example, a manifold) and nasal prongs, and/or a face mask, and/or a nasal pillows mask, and/or a nasal mask, and/or a tracheostomy interface, or any other suitable type of patient interface. The flow source could provide a base gas flow rate of between, e.g., 0.5 litres/min and 375 litres/min, or any range within that range, or even ranges with higher or lower limits. Details of the ranges and nature of flow rates will be described later.

A humidifier 52 can optionally be provided between the flow source 49 and the patient to provide humidification of the delivered gas. One or more sensors 48A, 48B, 48C, 48D such as flow, oxygen fraction, pressure, humidity, temperature, or other sensors can be placed throughout the system and/or at, on, or near the patient 56. Alternatively, or additionally, sensors from which such parameters can be derived could be used. In addition, or alternatively, the sensors 48A-48D can be one or more physiological sensors for sensing patient physiological parameters such as, heart rate, oxygen saturation, partial pressure of oxygen in the blood, respiratory rate, partial pressure of CO₂ in the blood. Alternatively, or additionally, sensors from which such parameters can be derived could be used. Other patient sensors could comprise electroencephalography (EEG) sensors, torso bands to detect breathing, and any other suitable sensors. In some configurations, the humidifier may be optional, or it may be preferred due to the advantages of humidified gases helping to maintain the condition of the airways. One or more of the sensors might form part of the apparatus, or be external thereto, with the apparatus having inputs for any external sensors. The sensors can be coupled to or send their output to a controller 19.

In some configurations, the respiratory apparatus 10 can include a patient sensor 29 for measuring the oxygen fraction of air the patient inspires. In some examples, the patient sensor 29 can be placed on the patient interface 17, to measure or otherwise determine the fraction of oxygen proximate (at/near/close to) the patient's mouth and/or nose. In some configurations, the output from the patient sensor 29 is sent to a controller 19 to assist control of the respiratory apparatus 10 and/or alter operation accordingly. The controller 19 is coupled to the flow source 49, humidifier 52, and the patient sensor 29. In some configurations, the controller 19 controls these and other aspects of the respiratory apparatus 10 as described herein. In some examples, the controller can operate the flow source 49 to provide the delivered flow of gas at a desired flow rate high enough to meet or exceed a user's (i.e., patient's) inspiratory demand. The flow rate is provided is sufficient that ambient gases are not entrained as the user (i.e., patient) inspires. In some configurations, the patient sensor 29 can convey measurements of oxygen fraction at the patient mouth and/or nose to a user, who can input the information to the respiratory apparatus 10 or controller 19.

An optional non-return valve 23 may be provided in the inspiratory conduit 16. A filter or filters may be provided at the air inlet 49C and/or inlets to the flow generator 49B to filter the incoming gases before they are pressurized into a high flow gas 27 by to the flow generator 49B.

The respiratory apparatus 10 could be an integrated or a separate component-based arrangement, generally shown in the dashed box 104 in FIG. 1. In some configurations, the apparatus could be a modular arrangement of components. Furthermore, the apparatus may just comprise some of the components shown; not necessarily all are essential. Also, the conduit and patient interface do not have to be part of the apparatus and could be considered separate. Hereinafter it will be referred to as a respiratory apparatus, but this should not be considered limiting. A ‘respiratory apparatus’ will be broadly considered herein to comprise anything that provides a flow rate of gas to a patient. Some such apparatus include a detection system that can be used to determine if the flow rate of gas meets inspiratory demand.

The respiratory apparatus 10 can include a main device housing which houses the components illustrated within the dashed box 104. The main device housing can contain the flow generator 49B that can be in the form of a motor/impeller arrangement, an optional humidifier or humidification chamber 52, a controller 19, and an input/output (I/O) user interface 54. The user interface 54 can include a display and input device(s) such as button(s), a touch screen (e.g., an LCD screen), a combination of a touch screen and button(s), or the like. The controller 19 can include one or more hardware and/or software processors and can be configured or programmed to control the components of the system, including but not limited to: operating the flow generator 49B to create a flow of gases for delivery to a patient, operating the humidifier or humidification chamber 52 (if present) to humidify and/or heat the gases flow, receiving user input from the user interface 54 for reconfiguration and/or user-defined operation of the respiratory apparatus 10, and outputting information (for example, on the display) to the user. The user can be a patient, healthcare professional, or others.

With continued reference to FIG. 1, an inspiratory conduit 16 can be coupled to a gases flow outlet (gases outlet or patient outlet port) 21 in a main device housing which houses the components illustrated within the dashed box 104 of the respiratory apparatus 10, and be coupled to a patient interface 17, such as a non-sealing interface like a nasal cannula with a manifold and nasal prongs. The inspiratory conduit 16 can also be a tracheostomy interface, or other unsealed interfaces.

The gases flow can be generated by the flow generator 49B, and may be humidified, before being delivered to the patient via the inspiratory conduit 16 through the patient interface 17. The controller 19 can control the flow generator 49B to generate a gases flow of a desired flow rate, and/or one or more valves to control mixing of air and oxygen or other breathable gas. The controller 19 can control a heating element in or associated with the humidification chamber 52, if present, to heat the gases to a desired temperature that achieves a desired level of temperature and/or humidity for delivery to the patient. The inspiratory conduit 16 can have a heating element, such as a heater wire, to heat gases flow passing through to the patient. The heating element can also be under the control of the controller 19.

The humidifier 52 of the apparatus is configured to combine or introduce humidity with or into the gases flow. Various humidifier 52 configurations may be employed. In one configuration, the humidifier 52 can comprise a humidification chamber that is removable. For example, the humidification chamber may be partially or entirely removed or disconnected from the flow path and/or apparatus. By way of example, the humidification chamber may be removed for refilling, cleaning, replacement and/or repair for example. In one configuration, the humidification chamber may be received and retained by or within a humidification compartment or bay of the apparatus or may otherwise couple onto or within the housing of the apparatus.

The humidification chamber of the humidifier 52 may comprise a gases inlet and a gases outlet to enable connection into the gases flow path of the apparatus. For example, the flow of gases from the flow generator 49B is received into the humidification chamber via its gases inlet and exits the chamber via its gases outlet, after being heated and/or humidified.

The humidification chamber contains a volume of liquid, typically water or similar. In operation, the liquid in the humidification chamber is controllably heated by one or more heaters or heating elements associated with the chamber to generate water vapour or steam to increase the humidity of the gases flowing through the chamber.

In one configuration, the humidifier is a pass-over humidifier. In another configuration, the humidifier may be a non-pass-over humidifier.

In one configuration, the humidifier may comprise a heater plate, for example, associated or within a humidification bay that the chamber sits on for heating. The chamber may be provided with a heat transfer surface, e.g., a metal insert, plate or similar, in the base or other surface of the chamber that interfaces or engages with the heater plate of the humidifier.

In another configuration, the humidification chamber may comprise an internal heater or heater elements inside or within the chamber. The internal heater or heater elements may be integrally mounted or provided inside the chamber or may be removable from the chamber.

The humidification chamber may be any suitable shape and/or size. The location, number, size, and/or shape of the gases inlet and gases outlet of the chamber may be varied as required. In one configuration, the humidification chamber may have a base surface, one or more side walls extending up from the base surface, and an upper or top surface. In one configuration, the gases inlet and gases outlet may be position on the same side of the chamber. In another configuration, the gases inlet and gases outlet may be on different surfaces of the chamber, such as on opposite sides or locations, or other different locations.

In some configurations, the gases inlet and gases outlet may have parallel flow axes. In some configurations, the gases inlet and gases outlet may be positioned at the same height on the chamber.

The respiratory apparatus 10 can use ultrasonic transducer(s), flow sensor(s) such as a thermistor flow sensor, pressure sensor(s), temperature sensor(s), humidity sensor(s), or other sensors, in communication with the controller 19, to monitor characteristics of the gases flow and/or operate the respiratory apparatus 10 in a manner that provides suitable therapy. The gases flow characteristics can include gases concentration, flow rate, pressure, temperature, humidity, or others. The sensors 48A, 48B, 48C, 48D, 29, such as pressure, temperature, humidity, and/or flow sensors, can be placed in various locations in a main device housing which also houses the components illustrated within the dashed box 104, the inspiratory conduit 16, and/or the patient interface 17. The controller 19 can receive output from the sensors to assist it in operating the respiratory apparatus 10 in a manner that provides suitable therapy, such as to determine a suitable target temperature, flow rate, and/or pressure of the gases flow. Providing suitable therapy can include meeting or exceeding a patient's inspiratory demand. In the illustrated embodiment sensors 48A, 48B, and 48C are positioned in the housing of the apparatus, sensor 48D in the inspiratory conduit 16, and patient sensor 29 in the patient interface 17.

The respiratory apparatus 10 can include one or more communication modules to enable data communication or connection with one or more external devices or servers over a data or communication link or data network, whether wired, wireless or a combination thereof. In one configuration for example, the respiratory apparatus 10 can include a wireless data transmitter and/or receiver, or a transceiver 15 to enable the controller 19 to receive data signals in a wireless manner from the operation sensors and/or to control the various components of the respiratory apparatus 10. The transceiver 15 or data transmitter and/or receiver module may have an antenna 15a as shown. In one example, the transceiver 15 may comprise a Wi-Fi modem. Additionally, or alternatively, the data transmitter and/or receiver 15 can deliver data to a remote patient management system (i.e., remote server) or enable remote control of the respiratory apparatus 10. The respiratory apparatus 10 can include a wired connection, for example, using cables or wires, to enable the controller 19 to receive data signals from the operation sensors and/or to control the various components of the apparatus 10. The respiratory apparatus 10 may comprise one or more wireless communication modules. For example, the apparatus may comprise a cellular communication module such as for example a 3G, 4G or 5G module. The module 15 may be or may comprise a modem that enables the apparatus to communicate with a remote patient management system (not illustrated in the figures) using an appropriate communication network. The remote management system may comprise a single server or multiple servers or multiple computing devices implemented in a cloud computing network. The communication may be two-way communication between the respiratory apparatus and a patient management system (e.g., a server) or other remote system. The respiratory apparatus 10 may also comprise other wireless communication modules such as, for example, a Bluetooth module and/or a Wi-Fi module. The Bluetooth and/or Wi-Fi module allow the apparatus to wirelessly send information to another device such as, for example, a smartphone or tablet or operate over a LAN (local area network) or Wireless LAN (WLAN). The apparatus may additionally, or alternatively, comprise a Near Field Communication (NFC) module to allow for data transfer and/or data communication.

For example, measured patient breathing parameter data (e.g., inspiratory, expiratory, and/or total respiratory time ratios) may be communicated to a remote patient management system (i.e., a remote server). The remote patient management system may be a single server or a network of servers or a cloud computing system or other suitable architecture for operating a remote patient management system. The remote patient management system (i.e., remote server) further includes memory for storing received data and various software applications or services that are executed to perform multiple functions. Then, for example, the remote patient management system (i.e., remote server) may communicate information or instructions to the respiratory apparatus 10 at least in part dependent on the data received. For example, the nature of the data received may trigger the remote server (or a software application running on the remote server) to communicate an alert, alarm, or notification to the respiratory apparatus 10. The remote patient management system may further store the received data for access by an authorized party such as a clinician or the patient or another authorized party. The remote patient management system may further be configured to generate reports in response to a request from an authorized party, and the breathing parameter data (e.g., inspiratory, expiratory and/or total respiratory time ratios) may be included into the generated reports. The reports may further comprise other patient breathing parameters (e.g., respiratory rate or SpO₂) and/or device parameters (e.g., flow rate, humidity level).

The respiratory apparatus 10 may comprise a high flow therapy apparatus. High flow therapy as discussed herein is intended to be given its typical ordinary meaning, as understood by a person of skill in the art, which generally refers to a respiratory apparatus delivering a targeted flow of humidified respiratory gases via an intentionally unsealed patient interface with flow rates generally intended to meet or exceed the peak inspiratory flow (also referred to as the inspiratory demand) of a user. Typical patient interfaces include, but are not limited to, a nasal or tracheal patient interface. Typical flow rates for adults often range from, but are not limited to, about fifteen litres per minute to about sixty litres per minute or greater. Typical flow rates for paediatric users (such as neonates, infants, and children) often range from, but are not limited to, about one litre per minute per kilogram of user weight to about three litres per minute per kilogram of user weight or greater.

High flow therapy can also optionally include gas mixture compositions including supplemental oxygen and/or administration of therapeutic medicaments.

High flow therapy is often referred to as nasal high flow (NHF), humidified high flow nasal cannula (HHFNC), high flow nasal oxygen (HFNO), high flow therapy (HFT), or tracheal high flow (THF), among other common names. For example, in some configurations, for an adult patient ‘high flow therapy’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 49 LPM and about 60 LPM. In some configurations, for a neonatal, infant, or child patient ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may deliver gases to the patient at a flow rate of between about 1 LPM and about 100 LPM, or at a flow rate in any of the sub-ranges outlined above.

High flow therapy can be effective in meeting or exceeding the patient's inspiratory demand, increasing oxygenation of the patient and/or reducing their work of breathing. Additionally, high flow therapy may generate a flushing effect in the nasopharynx such that the anatomical dead space of the upper airways is flushed by the high incoming gases flow. The flushing effect can create a reservoir of fresh gas available of each and every breath, while minimizing re-breathing of carbon dioxide, nitrogen, etc. High flow therapy can also increase expiratory time of the patient due to pressure during expiration. This in turn reduces the respiratory rate of the patient.

The patient interface for use in a high flow therapy can be a non-sealing interface to prevent barotrauma, which can include tissue damage to the lungs or other organs of the patient's respiratory system due to a difference in pressure relative to the atmosphere. The patient interface can be a nasal cannula with a gases flow part (for example a manifold) and nasal prongs, and/or an unsealed tracheostomy interface, or any other suitable type of patient interface.

The respiratory apparatus 10 can measure and control the oxygen content of the gas being delivered to the patient, and therefore the oxygen content of the gas inspired by the patient. The oxygen concentration measured in the respiratory apparatus 10, fraction of delivered oxygen, (FdO₂) may be substantially the same as the oxygen concentration the patient is breathing, fraction of inspired oxygen (FiO₂), and as such the terms may can be seen as equivalent.

The sensor 29 is a patient sensor. When the patient sensor 29 is disconnected (either from the patient or from the respiratory apparatus) during operation, the respiratory apparatus 10 may continue to operate in its previous state of operation for a predefined time period. After the predefined time period, the respiratory apparatus 10 may trigger an alarm, transition from automatic mode to manual mode, and/or exit control mode (e.g., automatic mode or manual mode) entirely.

The respiratory apparatus 10 may be configured to recognise whether the patient sensor 29 is a standalone patient sensor or a patient sensor that is located on or comprised by a patient interface 17. The respiratory apparatus 10 may recognise the sensor type by receiving identification information upon initial connection of the patient sensor 29. The respiratory apparatus 10 may recognise the sensor type through the way in which the signal from the patient sensor 29 is received. For example, an integrated patient sensor 29 may be configured to communicate with the respiratory apparatus 10 via an electrical connection located at the gases outlet of the respiratory apparatus 10 (as will be described herein), whilst a standalone patient sensor may be configured to connect to the respiratory apparatus via a separate connection port.

The patient sensor 29 may comprise other associated processors, or circuitry. Alternatively, the associated processors and/or circuitry may be located at another location.

The patient sensor 29 may comprise a housing. The housing may include the sensor transducer and any associated processors and/or circuitry.

The patient sensor 29 may be a skin contact sensor that contacts skin when in an operative position in the sensor cavity.

As described above, the patient sensor 29 may be a pulse oximeter sensor configured to measure a heart rate and/or blood oxygen saturation (SpO₂). The pulse oximeter sensor may be provided as part of a pulse oximeter to measure heart rate and/or blood oxygen saturation. A pulse oximeter may include the pulse oximeter sensor and processors and/or circuitry to control the pulse oximeter sensor by operating it and receiving sensor information from it. The processors and/or circuitry may be integrated with the pulse oximeter sensor or may be separate from it. The processors and/or circuitry may additionally process signals received from the pulse oximeter sensor and may communicate the processed signals or other resulting information to another device.

The connection between a pulse oximeter sensor and other processors and/or circuitry of a pulse oximeter may be wired and/or wireless, for example in the same manner as elsewhere described herein in relation to the potential configurations of a communication module of the respiratory apparatus 10. Where a pulse oximeter sensor is wirelessly connected with other processors and/or circuitry of the pulse oximeter, the pulse oximeter sensor may be powered by a wired connection or may be powered by a power source integrated with the pulse oximeter sensor for example a battery. In at least some configurations where the pulse oximeter sensor is wirelessly connected with other processors and/or circuitry of the pulse oximeter, a battery power source may be provided along with the pulse oximeter sensor. The battery power source may be user rechargeable.

The pulse oximeter sensor may be a reflectance pulse oximeter sensor.

The respiratory apparatus 10 may be configured to use the output of a patient sensor 29 that is located on or comprised by a patient interface 17 to determine whether the patient is wearing the patient interface 17. In this context, “wearing” refers to the patient interface 17 being mounted in a position on the patient's face such that the patient interface 17 can deliver the gases flow to the patient and the patient sensor 29 can measure one or more patient parameters. When the patient sensor 29 cannot reliably measure the one or more patient parameters it may produce a signal indicating such. Additionally, or alternatively, the patient sensor 29 may communicate a separate parameter such as signal quality. The respiratory apparatus 10 may check this parameter against a threshold in determining whether the patient sensor 29 is able to reliably measure the one or more patient parameters. The respiratory apparatus 10 may use a determination that the patient sensor 29 cannot reliably measure the one or more patient parameters in further determining that the patient is not wearing the patient interface 17.

The respiratory apparatus 10 may use the determination of whether the patient is wearing the patient interface 17 in activating or deactivating certain control algorithms, such as a closed-loop SpO₂ controller, which will be described in detail later in the specification. The respiratory apparatus 10 may use the indication in increasing or decreasing the flow rate. For example, the respiratory apparatus 10 may reduce the flow rate when the patient is not wearing the patient interface 17 in order to reduce noise and power consumption. The respiratory apparatus 10 may use the indication for generating an alarm, such as alarming if the patient has removed the patient interface 17. This alarm may occur instantaneously or within a set period of time after the output of the patient sensor 29 is lost.

In a further configuration, the respiratory apparatus 10 is configured to switch to a standby mode when the output of the patient sensor 29 indicates that the patient is not wearing the patient interface 17. In the standby mode, the respiratory apparatus 10 may be configured to control the blower to operate at a reduced motor speed. The reduced motor speed may be a minimum operating speed for the blower. The reduced motor speed may be about 1000 RPM-2000 RPM. In the standby mode, the respiratory apparatus 10 may be configured to control a blower to deliver a reduced flow rate. The reduced flow rate may be between about 1 LPM and 2 LPM.

The blower can operate at a motor speed of greater than about 1,000 RPM and less than about 30,000 RPM, greater than about 2,000 RPM and less than about 21,000 RPM, greater than about 4,000 RPM and less than about 19,000 RPM or between any of the foregoing values. Operation of the blower can mix the gases flow entering the blower through the inlet ports. Using the blower as the mixer can decrease the pressure drop that otherwise occurs in a respiratory apparatus with a separate mixer, such as a static mixer comprising baffles, because mixing requires energy. Having a static mixer can also increase the volume of the gas flow path between the valve and the gases composition sensor, which can further increase the delay between when the valve current is changed and when a corresponding change in oxygen concentration is measured.

Based on user inputs and the therapy supplied by the respiratory apparatus 10, the controller 19 can determine a target output parameter for the blower. The controller can receive measurements of the target output parameter and based on the difference between determined flow rate and the measured flow rate, the controller can adjust the speed of the blower.

With reference again to FIG. 1, the controller 19 can be programmed with or configured to execute a closed-loop control system for controlling the operation of the respiratory apparatus. The closed-loop control system can be configured to ensure the patient's SpO₂ reaches a target level and consistently remains at or near this level.

The controller 19 can receive input(s) from a user that can be used by the controller 19 to execute the closed loop control system. The target SpO₂ value can be a single value or a range of values. The value(s) may be pre-set, chosen by a clinician, or determined based on the type of patient, where type of patient may refer to current affliction, and/or information about the patient such as age, weight, height, gender, and other patient characteristics. The target SpO₂ value may be input by a clinician or user via a user interface on the apparatus and received by the controller 19. Similarly, the target SpO₂ may be two values, each selected in any way described above. The two values represent a range of acceptable values for the patient's SpO₂. The controller can target a value within said range. The targeted value may be the middle value of the range, or any other value within the range, which may be pre-set or selected by a user. Alternatively, the range may be automatically set based on the targeted value of SpO₂. The controller can be configured to have one or more set responses when the patient's SpO₂ value moves outside of the range. The responses may include alarming, changing to manual control of FdO₂, changing the FdO₂ to a specific value, and/or other responses. The controller can have one or more ranges, where one or more different responses occur as it moves outside of each range.

Generally, SpO₂ is controlled between about 80% and about 100%, or about 80% and about 90%, or about 88% and about 92%, or about 90% and about 99%, or about 92% and about 96%. The SpO₂ may be controlled between any two suitable values from any two of the aforementioned ranges. The target SpO₂ may be between about 80% and about 100%, or between about 80% and about 90%, or between about 88% and about 92%, or between about 90% and about 99%, or between about 92% and about 96%, or about 94%, or 94%, or about 90%, or 90%, or about 85%, or 85%. The SpO₂ target may be any value between any two suitable values from any two of the aforementioned ranges. The SpO₂ target can correspond to the middle of the SpO₂ for a defined range.

The FdO₂ can be configured to be controlled within a range. As discussed previously, the oxygen concentration measured in the apparatus (FdO₂) is substantially the same as the oxygen concentration the patient is inspiring (FiO₂) so long as the flow rate meets or exceeds the peak inspiratory demand of the patient, and as such the terms may can be seen as equivalent. Each of the limits of the range may be pre-set, selected by a user, or determined based on the type of patient, where the type of patient may refer to current affliction, and/or information about the patient such as age, weight, height, gender, and/or other patient characteristics. Alternatively, a single value for FdO₂ may be selected, and the range may be determined at least partially based on this value. For example, the range may be a set amount above and below the selected FdO₂. The selected FdO₂ may be used as the starting point for the controller. The system may have one or more responses if the controller tries to move the FdO₂ outside of the range. These responses may include alarming, preventing the FdO₂ moving outside of the range, switching to manual control of FdO₂, and/or switching to a specific FdO₂. The respiratory apparatus 10 may have one or more ranges where one or more different responses occur as it reaches the limit of each range.

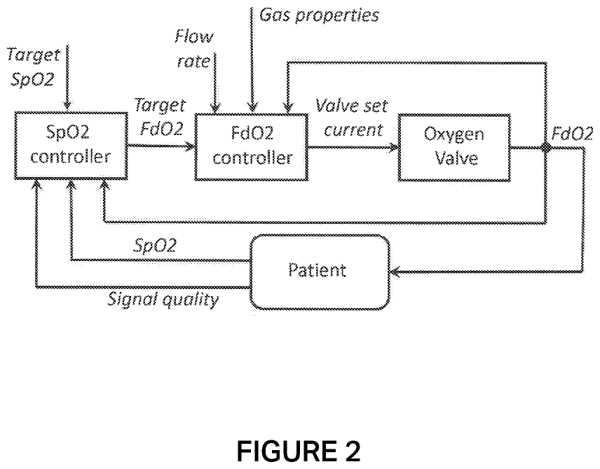

With reference to FIG. 2, a schematic diagram of the closed loop control system is illustrated. The closed loop control system may utilize two control loops. The first control loop may be implemented by the SpO₂ controller. The SpO₂ controller can determine a target FdO₂ based in part on the target SpO₂ and/or the measured SpO₂. As discussed above, the target SpO₂ value can be a single value or a range of acceptable values. The value(s) may be pre-set, chosen by a clinician, or determined automatically based on patient characteristics. Generally, target SpO₂ values are received or determined before or at the beginning of a therapy session, though target SpO₂ values may be received at any time during the therapy session. During a therapy session, the SpO₂ controller can also receive as inputs: measured FdO₂ reading(s) from a gases composition sensor, and measured SpO₂ reading(s) and a signal quality reading(s) from the patient sensor 29. In some configurations, the SpO₂ controller can receive target FdO₂ as an input, in such a case, the output of the SpO₂ controller may be provided directly back to the SpO₂ controller as the input. Based at least in part on the inputs, the SpO₂ controller can output a target FdO₂ to the second control loop.

During the therapy session, the SpO₂ and FdO₂ controllers can continue to automatically control the operation of the respiratory apparatus until the therapy session ends or an event triggers a change from the automatic mode to manual mode.

For example, a respiratory support system using blood oxygen saturation (SpO₂) measurements from a pulse oximeter to automatically adjust the fraction of oxygen of the gases flow being delivered to a patient (FdO₂) via a patient interface is described in the PCT application publication WO2019/070136 (herein “WO'136”) filed on 5 Oct. 2018 and hereby incorporated by reference in its entirety.

The respiratory support system described in WO'136 uses a separate pulse oximeter sensor and patient interface. As such, a clinician is required to attach both the pulse oximeter sensor and the patient interface to the patient individually, with both of these components also being connected separately to a respiratory apparatus.

With reference again to FIG. 1, the controller 19 can be programmed with or configured to execute an FdO₂ control system for controlling the operation of the respiratory apparatus.

The FdO₂ control system can be configured to ensure that the instantaneous FdO₂ is maintained at a target level at all points during a therapy session. The controller can measure the FdO₂, compare it with the target FdO₂, and then adjust the oxygen inlet valve accordingly. However, when the FdO₂ sensors are located at a non-insignificant distance away from the valve, there is a time delay between when a change is made to the valve and when a corresponding change in the FdO₂ is measured. The controller may adjust the valve after the time delay. However, if the flow rate is fluctuating, then the controller may be able to achieve the target FdO₂ on average, but not at a continuous and substantially instantaneous basis. As shown in FIG. 2, in order to maintain the FdO₂ at the target level at a continuous and substantially instantaneous basis, without moving the FdO₂ sensors closer to the valve, the FdO₂ controller can factor in the measurement of a total flow rate into the control of the valve.

A patient interface 17 is connected to one end of the inspiratory conduit 16 and is used to provide a breathable gases flow to the patient. During setup of the respiratory apparatus 10, a clinician or the patient is required to attach the patient interface 17 to the patient. Additionally, if a standalone patient sensor 29 is also to be used, then the clinician or the patient are also required to attach this to the patient. Both of the patient interface 17 and the patient sensor 29 then also need to be attached to the respiratory apparatus 10 itself. Forming these various connections can be undesirable.

The patient interface 17 has one or more patient sensors 29. The one or more integrated patient sensors 29 may be configured to measure the blood oxygen saturation (SpO₂) of the patient. The one or more integrated patient sensors 29 are positioned on the patient interface 17 to facilitate the measuring of the patient's blood oxygen saturation (SpO₂).

The patient interface 17 may be used with the respiratory apparatus 10 described above. Alternatively, the patient interface 17 may be used with any other respiratory apparatus that may utilize a patient interface 17 with patient sensors 29, such as a ventilator, a CPAP apparatus, a standalone humidifier, and/or an oxygen blender.

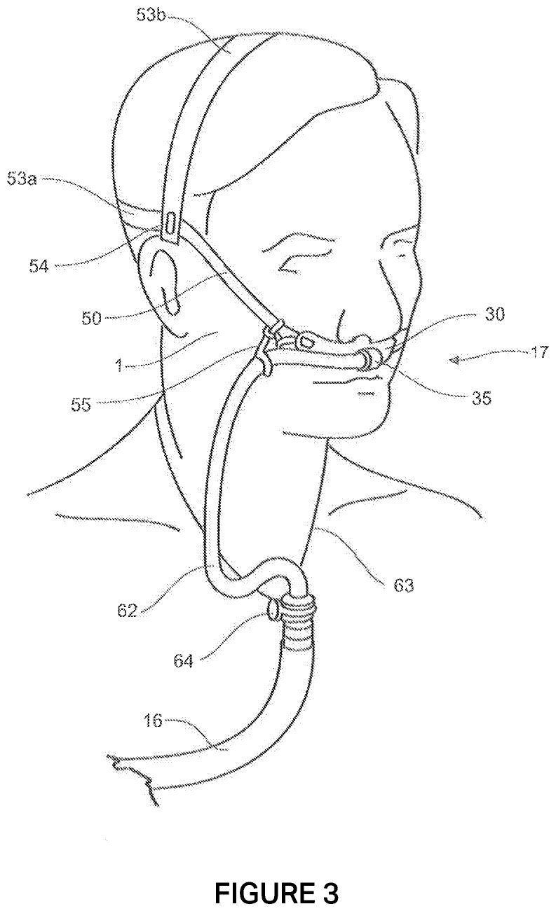

The patient interface 17 may comprise a nasal cannula interface, as shown in FIGS. 3 to 7. In this configuration, the nasal cannula interface broadly comprises a head securement assembly (otherwise referred to as a headgear) and a nasal cannula 30, and also includes a gases inlet conduit 62. The head securement assembly enables a user to place and maintain the nasal cannula 30 in the correct operational position. The gases inlet conduit 62 forms a fluid or gases connection between the outlet end of the inspiratory conduit 16 and the nasal cannula 30 to allow fluids or gases to flow between the inspiratory conduit and nasal cannula. The gases inlet conduit 62 and detail of the main portion of the nasal cannula 30 will be described in detail below.

The head securement assembly of the nasal cannula 30 may comprise one or more straps. The one or more straps may include two front straps 50, a rear strap 53a, and a top strap 53b, as shown in FIG. 3. In some configurations, the proximal end of the front straps 50 are removably connected to the nasal cannula 30. In other configurations, the proximal ends of the front straps 50 are non-removably connected to the nasal cannula 30. The rear strap 53a and the top strap 53b extend between the distal ends of the front straps 50. In use, the rear strap 53a wraps around the back of the patient's head. In use, the top strap 53b wraps around the top of the patient's head. In some configurations the head securement assembly is adjustable to allow patients of different head shapes and sizes to use the nasal cannula 30. For example, an adjuster such as an adjustment buckle 54 may be included which allows a patient to loosen or tighten the top strap 53b.

In some configurations, one or more of the straps are substantially elastic (i.e., made from an elastic material, e.g., elastane, that can stretch to accommodate a patient's head). In some configurations, one or more of the straps are substantially rigid. In some configurations, one or more of the straps are made of a substantially rigid material. In some configurations, one or more of the straps are substantially inextensible. In some configurations, one or more of the straps are made of a substantially inextensible material. In some configurations, one or more of the straps are self-supporting. In some configurations, one or more of the straps maintain their shape when not in use.

Alternatively, the patient interface 17 is secured to the patient's head and face by front straps 50 and a single rear strap 53a attached to the front straps 50. The rear strap is attached to the front straps 50 via a buckle 54. Alternatively, the rear strap 53a is integral with the front straps 50. The buckle 54 allows a patient to loosen or tighten the front straps 50 based on individual preference. Alternatively, the integral front 50 and rear straps 53a are elastic and can be stretched over a patient's head. The elasticity of the straps exerts a force upon the head to hold the nasal cannula 30 in the optimal position when in use. Elastic front straps 50, 53a can be used with the adjustment buckle 54 or the elastic front straps 50, 53a may be used on their own without the buckle 54.

The head securement assembly may also include a loop 55 which holds and supports the gases inlet conduit 62 at or close to the inlet end as shown in FIG. 3. The loop 55 comprises a first end connected to one of the front straps 50. The first end may be slidably connected to the front strap 50. The loop 55 comprises a second end connected to the gases inlet conduit 62. The second end may be removably connected to the gases inlet conduit 62. Alternatively, the interface may comprise a tube clip that is connected to the tube and can be removably coupled to the cannula. The tube clip supports the weight of the gases inlet conduit 62 and reduces the moment caused by the gases inlet conduit 62, thereby improving stability of the patient interface 17. The clip helps to reduce dislodgement of the patient interface 17. The clip may be formed of a rigid material.

A lanyard 63 may also be provided with the patient interface 17. FIG. 3 shows an example of a lanyard 63. In the configuration shown, the lanyard 63 is connected to the gases inlet conduit 62. Alternatively, the lanyard 63 is connected at a location at or close to the connection between the inspiratory conduit 16 and the gases inlet conduit 62. In use, the lanyard 63 supports the weight of the inspiratory conduit 16 and the gases inlet conduit 62. A toggle 64 is provided with the lanyard 63 to allow adjustment of the lanyard's length. The toggle 64 makes the lanyard 63 suitable for any sized patient to use the patient interface 17. The lanyard 63 supports at least a portion of the weight of the inspiratory conduit 16 in use, such that the weight does not act on the patient or the nasal cannula 30. The use of the lanyard 63 reduces the portion of the combined weight of the inspiratory conduit 16 and the gases inlet conduit 62 that pulls on the nasal cannula 30, helping to prevent the nasal prongs 33, 34 from interfering with the sensitive lining of the nasal passages, or becoming displaced or misaligned in use. In the configuration shown the lanyard 63 is loose fitting around the neck so as to reduce the chance of strangulation of the patient. The lanyard 63 also provides a convenient way of supporting the inspiratory conduit 16 and the gases inlet conduit 62. This allows the patient to turn in bed without tugging or pulling on the inspiratory conduit 16 and helps avoid having the gases inlet conduit 62 from overheating under the blankets. In one configuration the lanyard 63 has a clip that allows the lanyard to be opened and closed by a user in order place and secure the lanyard 63 around a ‘patient’s neck. The clip comprises a male and female connector that snap fit together. The clip is disconnected by pulling one end of the lanyard 63. The clip is easily disconnectable and uncouples when the user pulls on one side of the lanyard. This allows the lanyard 63 to be removed quickly, for example in an emergency situation, such as if the patient needs to be intubated.

The gases inlet conduit 62 will now be described in detail. The gases inlet conduit 62 is a short length of conduit or tubing relative to the inspiratory conduit 16 which runs between the outlet of the inspiratory conduit 16 and the nasal cannula 30. In use, the gases inlet conduit 62 forms a lumen that defines a gases pathway between the inspiratory conduit 16 and the patient interface 17, such that the gases flow exits the inspiratory conduit 16 and enter the gases inlet conduit 62, travelling along the gases inlet conduit 62 to the patient interface 17 to be delivered to the patient. One reason that secondary conduits such as the gases inlet conduit 62 can be used is as follows: the inspiratory conduit 16 is relatively heavy and cumbersome as it is used to transport the gases flow over a reasonably long distance (from the humidifier unit 2 to a point close to the patient). The inspiratory conduit 16 is therefore required to have a wall that is strong enough to support its own weight without collapsing. As the inspiratory conduit 16 is typically relatively long (e.g., 8 to 10 feet), this additional length and the thicker wall structure adds to the weight of the inspiratory conduit 16. If the outlet of the inspiratory conduit 16 is connected directly to the patient interface in such a manner that the patient is required to support this weight, this can cause discomfort to the patient due to the weight of inspiratory conduit 16 acting on the patient. Furthermore, the weight of the inspiratory conduit 16 can pull on the patient interface 17 and cause it to become dislodged or misaligned. A lighter, shorter secondary conduit (e.g., gases inlet conduit 62) running between the outlet of the inspiratory conduit 16 and the patient interface 17 can be used.

Gases inlet conduit 62 is lighter and shorter than the inspiratory conduit 16, and as outlined above, is generally used with e.g., a lanyard 63 connected to the gases inlet conduit 62 or to the connection between the inspiratory conduit 16 and the gases inlet conduit 62. In use, the lanyard 63 (as outlined above) supports at least a portion of the weight of the inspiratory conduit 16, such that the patient interface 17 only needs to support the comparatively lighter the gases inlet conduit 62. Furthermore, in configurations in which the lanyard 63 connects to the end of the gases inlet conduit 62, the patient does not need to remove the lanyard 63 when disconnecting the gases inlet conduit 62 from the inspiratory conduit.

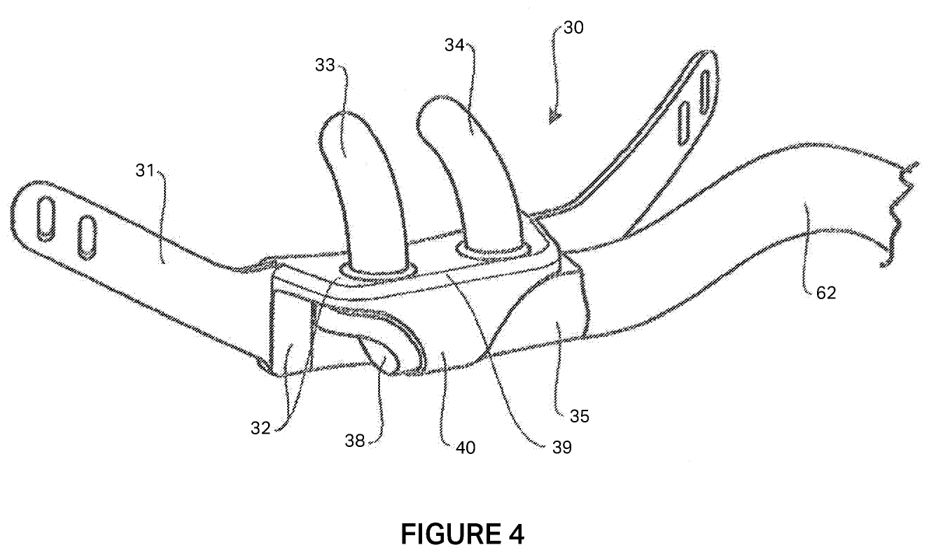

Various aspects of the nasal cannula 30 shall now be described in more detail with reference to FIGS. 4 to 6B. Unless otherwise stated, the nasal cannula 30 illustrated in FIGS. 4 to 6B includes all of the features of the generalized nasal cannula described with reference to FIG. 3.

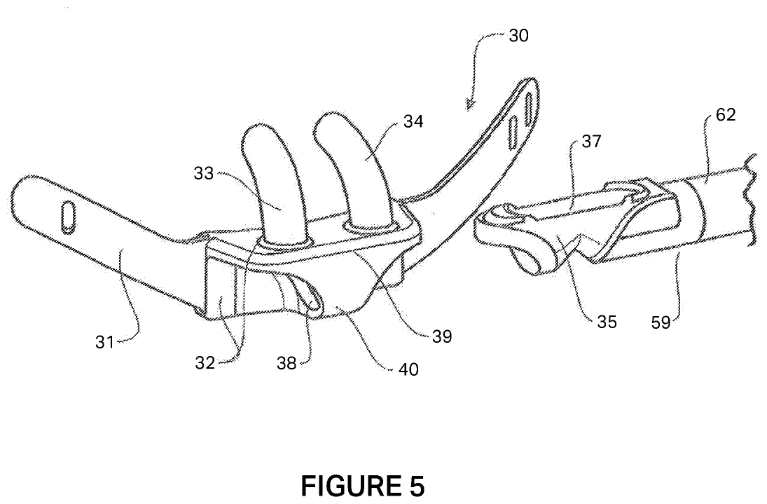

The nasal cannula 30 comprises two main parts: an interface connector 35 and a body 32. Example configurations of these two parts will now be described with particular reference to FIGS. 4 and 5.

The interface connector 35 is in use connected to and in fluid communication with the gases inlet conduit 62 as has been described above. However, it may be connected directly to the inspiratory conduit 16 in alternative embodiments.

The configuration of FIG. 5 shows the interface connector 35 as being detachable from the remainder of the nasal cannula 30. Alternatively, the interface connector 35 may be an integral part of the nasal cannula 30. Alternatively, the interface connector 35 and the nasal cannula 30 form a one-time fit, such that the user is prevented from disassembling the two components following the initial assembly. In the integrated or one-time fit configurations, a continuous gases flow path is formed through the inspiratory conduit 16, the gases inlet conduit 62, the interface connector 35, and to the prongs of the nasal cannula 30.

In some configurations interface connector 35 is generally tubular in shape having a substantially circular inlet 59 on one side that curves to an oval or elliptical outlet 37, the outlet 37 being formed on one side of the interface connector 35 so that it is perpendicular to the inlet 59. The circular inlet 59 in the illustrated form receives the patient end of the gases inlet conduit 62, such that the gases flow from the gases inlet conduit 62 can pass through the interface connector 35.

In some configurations the interface connector 35 is integrated with or permanently coupled to the gases inlet conduit 62. Alternatively, the interface connector 35 is removably attached to the gases inlet conduit 62. The interface connector 35 engages with the body 32 so that the gases flow can pass through the outlet 37 and transfer from the gases inlet conduit 62 to the patient through the nasal prongs 33, 34 (described in detail below).

In some configurations the interface connector 35 is manufactured from a hard plastic material that only deforms under relatively high loading conditions (that is, it cannot easily be crushed in the hand of a user). The interface connector 35 may be moulded, injection moulded, machined, or cast.

The interface connector 35 in use is connected to the body 32, so that the gases flow exiting the interface connector 35 enter the body 32. The body 32 will now be described in detail.

The body 32 includes the nasal prongs 33, 34 extending from a base portion 39 of the body 32. The gases flow passes through the body 32 to the nasal prongs 33, 34 and is delivered to the patient. In some configurations, the nasal prongs 33, 34 extend parallel to each other. In some configurations, the nasal prongs 33, 34 curve rearwards from the face mount portion or body 32. In some configurations, the nasal prongs 33, 34 curve towards each other. The structure of the prongs 33, 34 will be described in detail below.

The body 32 of the illustrated embodiment comprises side arms 31 and a tubular member 38 comprising a recess, integrally moulded together as shown in FIGS. 4 and 5. The tubular member 38 extends below the body 32 and is adapted to receive the interface connector 35 (for the configurations where the body 32 and the interface connector 35 are separable or separate items). The body 32 has a lip 39 that extends around the upper edge of the tubular member 38. The interface connector 35 is connected to the body 32 by a friction fit and the lip 39 on the body 32 helps to grip the interface connector 35 and form a sealed connection between the interface connector 35 and the body 32. The tubular member 38 comprises a rib 40 which extends below the body 32. The rib 40 helps to cradle and hold the interface connector 35 in the correct position as it engages with the body 32, the rib 40 extending around the outside of the interface connector 35. Outlet 37 on the interface connector 35 aligns in use with the underside of the face mount portion or body 32 when the interface connector 35 is connected to the body 32. This alignment reduces the amount of gases which leak out of the nasal cannula 30, allowing effective treatment of the patient by delivering maximum amount of humidified gases.

The side arms 31 are used to attach the front straps 50 to the body 32. The side arms 31 extend from either side of the body 32. In some configurations, the side arms 31 are formed as an integral part of the body 32. In use, the front straps 50 are attached to the side arms 31 so that the patient interface can be worn by a patient. In some configurations the ends of the front straps 50 are looped through a pair of slits on the side arms 31, with the ends including a hook and loop fastener or similar to hold the ends in place when they are looped back on themselves. Alternatively, the front straps 50 or loops 66 may be clipped onto the side arms 31, for example by way of co-operating male-female clips, or adhesively attached to the side arms 31.

In some configurations, the body 32, nasal prongs 33, 34, side arms 31 and the tubular member 38 are all manufactured as one continuous item. The body 32, nasal prongs 33, 34, side arms 31 and the tubular member 38 are all manufactured out of flexible polymer material such as a soft thermoplastic elastomer (TPE), or silicone.

The following is a description of the nasal prongs. In the following description the term “rear”, or “back” or any such synonym refers to that part of the structure that faces towards and is closest to the patient's face when the nasal cannula is in use. The term “front” or “forward” or any such synonym refers to the side, face or part which faces away from and is furthest away from the face of a patient in use. The term “top” or “upper” refers to the side, face or part that is pointing away from the floor, when a patient wearing the patient interface is standing or sitting upright and looking forward. The term “bottom” or “lower” refers to the side, face or part that is directed or pointing toward the ground, again when a patient wearing the interface is standing or sitting upright and looking forward. For example, FIG. 3 illustrates the patient interface 17 being worn by the patient, wherein the directions described above can be evaluated with reference to this Figure. The definitions for these directions remain consistent throughout, including in figures where the patient interface 17 is shown without the patient.

In some configurations the body 32 includes two nasal prongs 33, 34 extending upwards and curving inwards from the upper surface of the body 32 as shown in FIGS. 4 to 6B. Referring to FIGS. 4 to 6B, the nasal prongs 33, 34 extend from the upper surface of the body 32 and each prong is placed in each nostril of the patient when the nasal cannula is in use. The prongs 33, 34 are configured to deliver the gases flow to a patient. The prongs 33, 34 receive the humidified gases flow from the gases inlet conduit 62 via the gases inlet conduit 62, the interface connector 35 and the body 32. The nasal prongs 33, 34 are therefore in fluid connection with the interface connector 35 and receive the gases flow from the gases inlet conduit 62.

Referring to FIGS. 7A and 7B, the patient sensor 29 is located on the body 32 of the nasal cannula 30. In some configurations, the patient sensor 29 is located on the nasal cannula 30 such that it contacts the patient's skin during use. The patient sensor 29 may have an adhesive surface such that it can be secured in contact with the patient's skin.

The outer surfaces of the body 32 of the nasal cannula 30 may generally be divided into outwardly facing surfaces and inwardly facing surfaces. The term “outwardly facing surface” used herein can refer to an outer surface of the body 32 that faces away from the patient whilst the nasal cannula 30 is in use. The term “inwardly facing surface” used herein can refer to an outer surface of the body 32 that faces towards from the patient whilst the nasal cannula 30 is in use. The front and bottom sides of the body 32 can be considered to be outwardly facing surfaces, whilst the rear side can be considered to be an inwardly facing surface. The central portion of the top side of the body 32 that is located under the patient's nose in use may be considered to be an inwardly facing surface, whilst the remaining side portions of the of the top side may be considered to be outwardly facing surfaces.

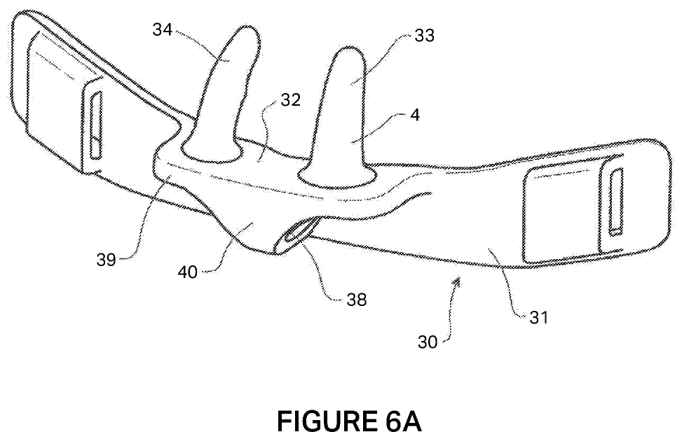

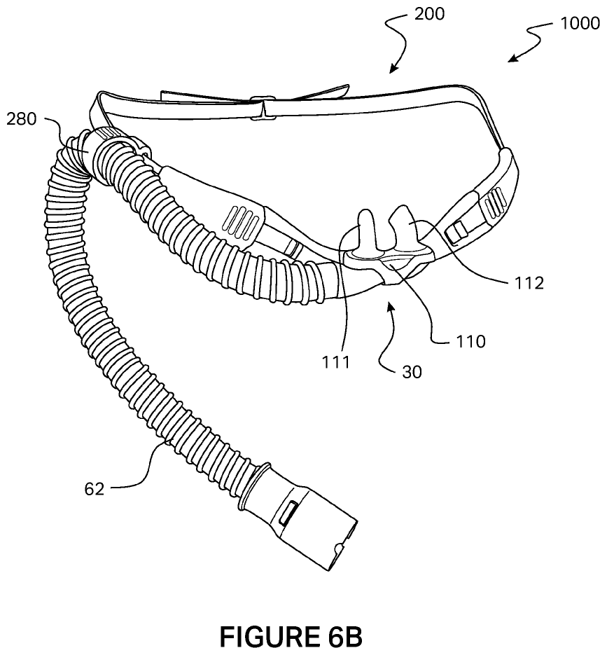

FIG. 6B shows an exemplary patient respiratory interface 1000 that comprises a nasal cannula 30 with asymmetrical nasal delivery elements 111, 112.

The nasal cannula 30 provides a patient with a patient interface suitable for the delivery of high airflow, high humidity gas flow to the patient's nasal cavity/nares. In some configurations, the nasal cannula 30 is adapted to deliver a high flow of gases over a wide flow range (e.g. about 8 1 pm, or higher depending on other therapy applications, perhaps such as 10-50 lpm or higher). In some configurations, the nasal cannula 30 is adapted to deliver relatively low-pressure gases.

As seen in FIG. 6B, a gases inlet conduit 62 passes inlet gases to the nasal cannula 30. A headgear 200 is shown provided with the patient respiratory interface 1000 to retain nasal cannula 30 on the patient's face in use. A retention clip 280 is shown retaining the gases inlet conduit 62 to the headgear 200. The nasal prongs 111, 112 are associated with a main body 110 of the nasal cannula 30.

The nasal prongs 111 and 112 are curved to extend into the patient's nares in use and to provide a smooth flow path for gases to flow through. The inner surfaces of the prongs 111 and 112 may be contoured to reduce noise. The bases of the prongs 111 and 112 may include curved surfaces to provide for smoother gases flow. This may reduce the noise level during operation.

The nasal prongs 111 and 112 are substantially hollow and substantially tubular in shape.

The nasal prongs 111 and 112 may be consistent in diameter along their lengths or alternatively may be shaped to fit the contours of the nares.

The main body 110 is shaped to generally follow the contours of a patient's face around the upper lip area. The face mount part 110 is moulded or pre-formed to be able to conform to and/or is pliable to adapt, accommodate and/or correspond with the contours of the patient's face, in the region of the face where the cannula is to be located.

The asymmetry of the nasal prongs 111 and 112 may reduce the chance of accidental occlusion of both nares. At least one of the nasal prongs 111 and 112 is therefore sized to maintain a sufficient gap between the outer surface of the prongs 111 and 112 and the patient's skin to avoid sealing the gas path between the nasal cannula 30 and patient. It should be understood that in the context of the present disclosure, the nasal prongs 111 and 112 are asymmetric, as described below.

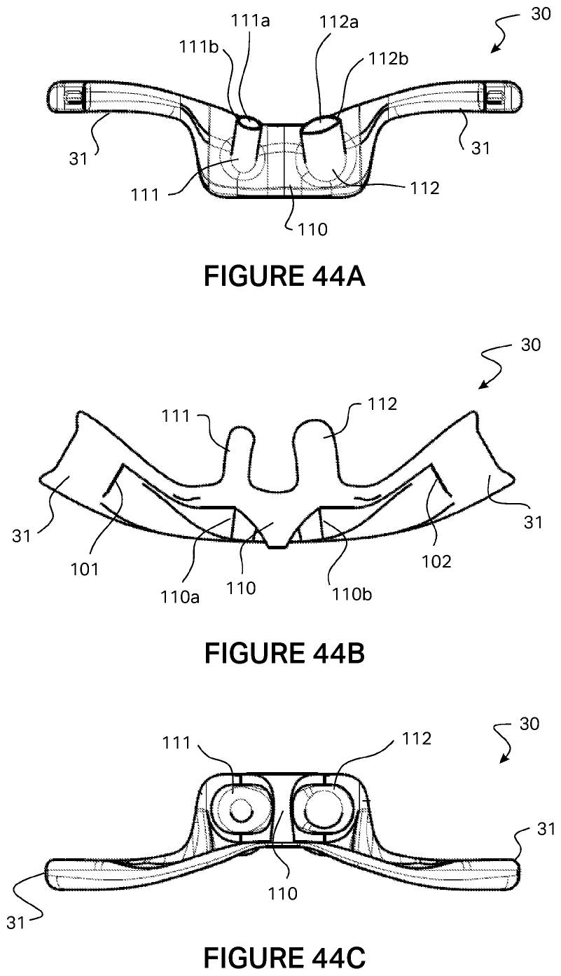

A further example of a patient interface having asymmetric prongs is shown in FIGS. 44A-C.

As seen in FIG. 6A and in FIGS. 44A-C, in some configurations a nasal cannula 30 of the present disclosure comprises a first prong 111 and a second prong 112 that are asymmetrical to each other, and a main body 110 comprising a gases inlet. The first prong 111 and the second prong 112 are in fluid communication with the gases inlet. The nasal cannula is configured such that at least about 60% of a total volumetric flow rate of gases flow into the gases inlet is delivered out of the nasal cannula through the second prong 112.

The first prong 111 and the second prong 112 as illustrated in FIG. 6B and FIGS. 44A-C can be considered asymmetrical nasal delivery elements.

The first prong 111 and the second prong 112 are asymmetrical to each other and/or are not symmetrical to each other and/or differ in shape and configuration to each other and/or are asymmetrical when compared to each other.

The nasal cannula 30 is configured to cause an asymmetrical flow of gases at, into and/or out of a patient's nares.

In some configurations, the nasal cannula 30 comprises a main body 110 comprising the first prong 111 and the second prong 112.

In some configurations, the first and second prongs 111, 112 are configured to engage with the nasal passages in an unsealed (non-sealing) manner. In some configurations, at least the second prong 112 is configured to engage with a nasal passage in a non-sealing manner.

In some configurations, the first and second prongs 111, 112 allow exhaled gases to escape around the first and second prongs.

In some configurations, the first and second prongs 111, 112 are configured to provide gases to the patient without interfering with the patient's spontaneous respiration.

The first prong 111 has a first prong outlet 111a defined by an opening at its tip or terminal end 111b for delivery of gases from the first prong 111. Gases delivered through the first prong 111 exit the first prong via the first prong outlet 111a.

The second prong 112 has a second prong outlet 112a defined by an opening at its tip or terminal end 112b for delivery of gases from the second prong 112. Gases delivered through the second prong 112 exit the second prong via the second prong outlet 112a.

We refer now to FIGS. 7A to 9C. These embodiments show or show parts of a patient interface 17 configured to deliver breathing gases from a gases supply and humidification source (not shown) to the patient, and headgear 200 configured to support and retain the patient interface against the patient's face in use. As seen, for example, in FIGS. 7A and 7B, the patient interface 17 is in the form of a nasal cannula interface 1000 that is adapted to couple the inspiratory conduit 16 via the gases inlet conduit 62 and that comprises at least one, but preferably two, nasal prongs 111 and 112 configured to fit within the nares of a patient to deliver a flow of gases to the patient. The headgear 200 is in the form of a head strap 200 that is preferably adjustable in length to customise the size of the strap to the patient.

The nasal cannula interface 1000 comprises a main body 110 including at least one, but preferably a pair of, tubular nasal prongs 111 and 112, integrally moulded with or removably attached to the main body 110 (i.e., body), and a gases flow part 120 that is removably attached or integrally moulded to the gases inlet conduit 62. The gases flow part 120 is insertable into the main body from either one of two opposing horizontal directions, i.e., from either left side or the right side. In this manner, the position or location of the gases flow part 120 is reversible with respect to the main body 110 (i.e., body). In other words, a user may choose to have the gases flow part 120 (and essentially the gases inlet conduit 62 extending there-from) extend from either the left side or the right side of the nasal cannula interface 1000 depending on what is most convenient, for example depending on which side of the patient the gas source or ventilator is located.

The gases flow part 120 may be configured to fluidly couple the prongs of the patient interface 1000 to the gases inlet conduit 62. In some embodiments, the gases flow part 120 may be a manifold.

The main body 110 is formed from a soft and flexible material such as silicone or other cannula material known in the art. The nasal prongs 111 and 112 are preferably supple and maybe formed from a sufficiently thin layer of silicone to achieve this property.

The gases flow part 120 is formed from a relatively harder material such as a polycarbonate, a high-density polyethylene (HDPE) or any other suitable plastics material known in the art. The main body 110 provides a soft interfacing component to the patient for comfortably delivering the flow of gases through the nasal prongs 111 and 112, while the gases flow part 120 fluidly couples the gases inlet conduit 62 to the nasal prongs 111 and 112 of the main body 110.

The patient sensor 29, such as a pulse oximeter sensor or multiple pulse oximeter sensors, may be located on or in the gases flow part 120.

The patient sensors 29 may be integrated into the gases flow part 120 and therefore may be disposable. Alternatively, the patient sensors 29 may be removably mounted on the gases flow part 120. The gases flow part 120 may have an appropriate recess or receiving port/opening to receive the one or more patient sensor(s) 29. The one or more patient sensor(s) 29 may be removable and reusable.

As previously described, the one or more patient sensor(s) 29 may be wireless and/or wired. The or one or more wires of the patient sensor(s) 29 can be routed through the gases flow part 120, via the inlet and back to a controller via the inspiratory conduit 16 or 62. The controller may include the circuitry to operate the patient sensors, for example one or more pulse oximeter sensors. The controller which controls the patient sensors may be a separate controller to a controller 19 which controls the respiratory apparatus. In other arrangements, the controller 19 which controls the respiratory apparatus may also control the patient sensors.

The one or more sensor(s) are positioned on the gases flow part 120 so as to position the patient sensor(s) 29 in contact with or adjacent the upper lip region, e.g., in the oral region of the face. There are a number of blood vessels in the upper lip and the patient sensor(s) 29 can be used to determine blood oxygen via contact with or proximity to the upper lip region via the gases flow part 120.

The gases flow part 120 may be formed from a rigid plastics material as it is received into a soft silicone body of the cannula. The gases flow part 120 being rigid makes it easier to insert the manifold part into the face mount portion and retain the manifold part in its operative position (i.e., inserted within the face mount). The manifold part is inserted into the main body and in fluid communication with the prongs to direct gases from the inlet conduit to the prongs. The patient sensors 29 located on or in the manifold part, being positioned in the main body, positions the patient sensor 29 in a sensing position—i.e., the sensor is positioned adjacent or in contact with the upper lip.

A patient's septum and/or columella is generally quite a sensitive area and can be a source of discomfort when subjected to excessive contact pressure for prolonged periods. The nasal cannula of the present disclosure can alleviate or reduce this pressure by providing a cushioned region of the nasal cannula interface 1000 adjacent the patient's septum/columella.

The patient sensor 29, for example a pulse oximeter sensor, may be placed in between the nasal prongs 111, 112 on the upper surface of the cannula such that it contacts the septum/columella.

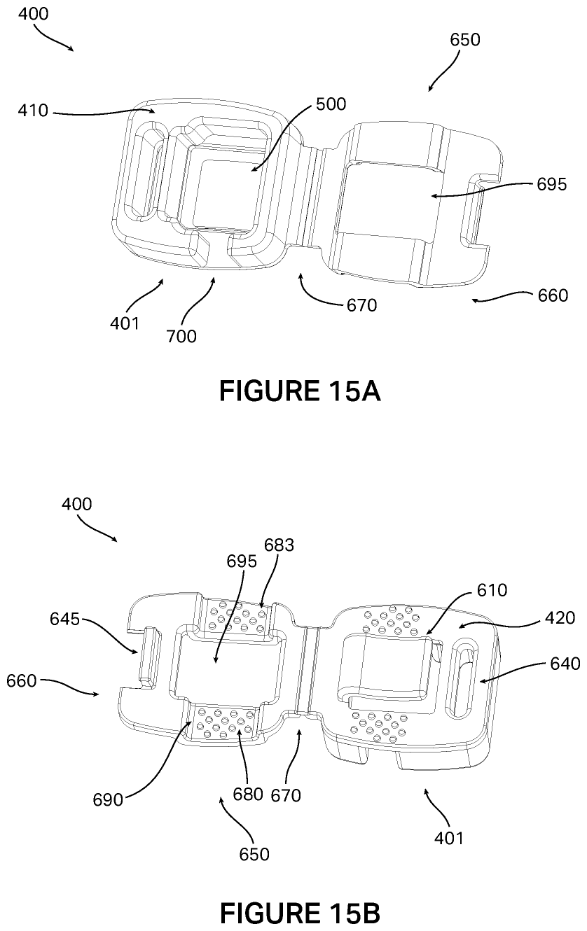

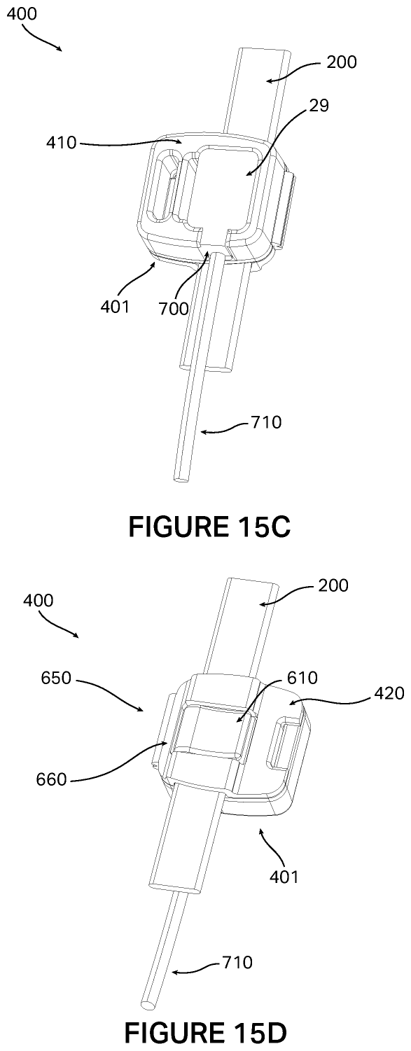

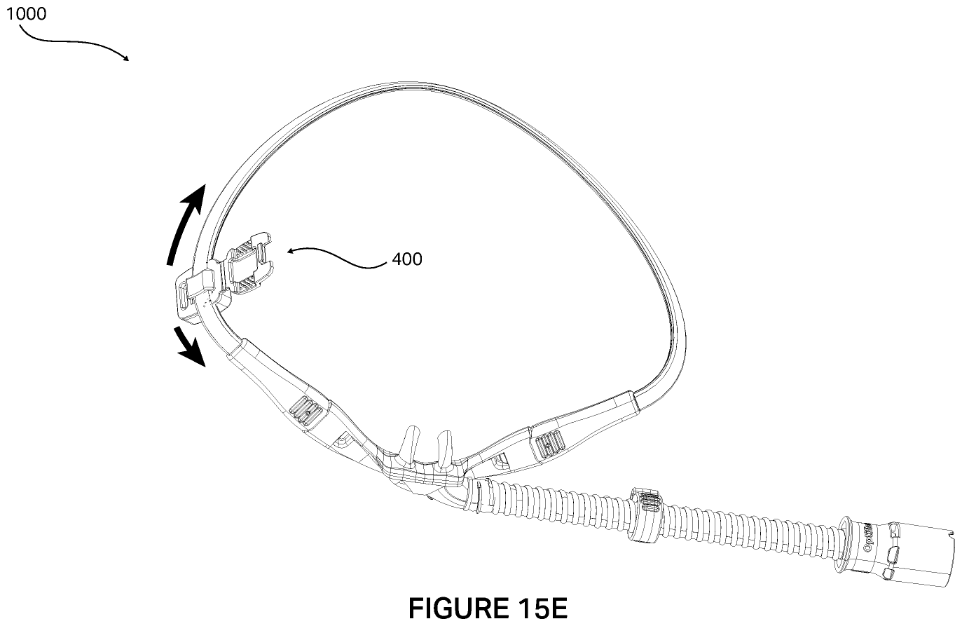

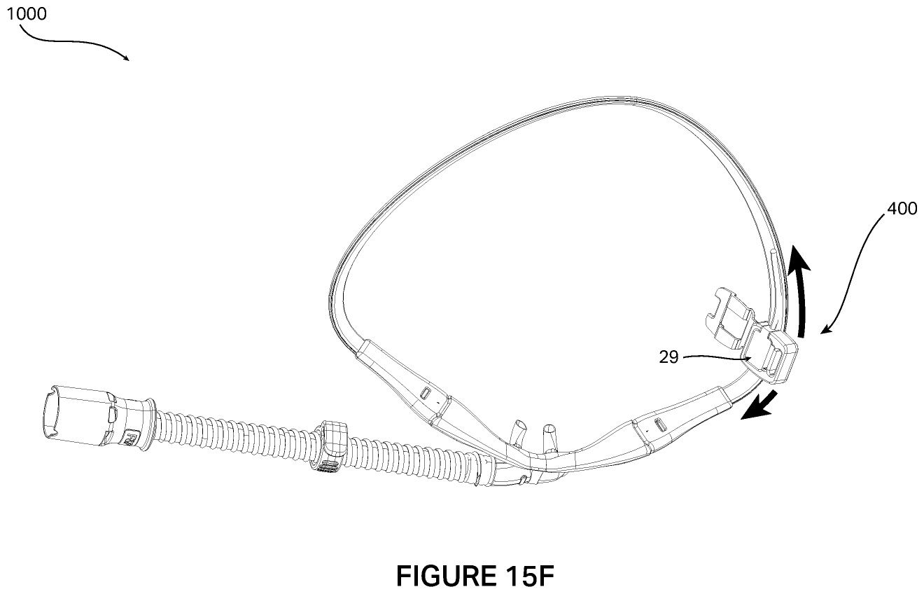

The patient sensor 29, for example a pulse oximeter sensor, may be located in an accessory 400 (for example as a nasal cannula interface accessory) and/or a nasal cannula component as described in more detail below. As shown in FIGS. 15C and 15F for example, the pulse oximeter sensor 29 is located in the accessory 400.

In the embodiment of FIGS. 7A to 9C, the headgear used to retain the nasal cannula interface 1000 against the patient's face comprises a head strap 200 of a single continuous length and is adapted to extend in use along the patient's cheeks, above the ears and about the back of the head.

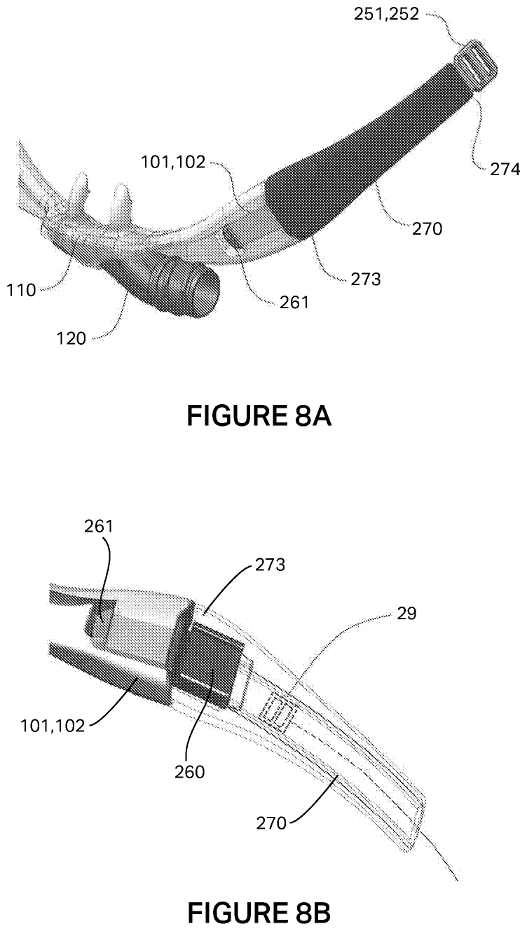

Primary end portions 201 and 202 of the strap 200 are adapted to releasably connect to respective formations 101 and 102 (see FIG. 8A for example) on either side of the nasal cannula 100 to hold the cannula 100 in position during use.

A strap connector 230 is provided at each of the secondary end portions 203/204 of the main strap 210 and the respective end portions 203/204 of the strap segment 220.

FIG. 7C shows a headgear of a nasal cannula interface where the strap segment 220 of FIG. 7B is provided as three differently sized strap segments 220a, 220b, and 220c. Each of the differently sized strap segments respectively have respective end portions 221a, 221b, and 221c, which correspond to the end portions 203 and 204 of the main strap 210.

Each connector 230 is provided with a strap connection mechanism at one end to couple to the strap material, and a coupling mechanism at an opposing end to releasably couple the respective end of a similar connector 230.

Cannula connectors 240 are provided at the primary end portions 201 and 202 of the main strap 210. These connectors 240 have a similar strap connection mechanism to the strap connectors 230 of the secondary end portions 203 and 204, but include a clip member, such as a push fit clip 241, at an end of the connector 240 opposing the strap ends. The clip 241 is configured to releasably couple the respective formation 101/102 at the side of the nasal cannula interface 1000. The clip 241 is preferably a bendable part, such as a plastic part, that forms a hinged portion relative to the strap. The clip 241 is preferably preformed to have a curved shape along its length, such as one with an angle between flat and 20 degrees for example. This curve allows the clip 241 to fit the contour of the patient's face in the region of the clip 241.

Referring to FIGS. 7D to 7H, a method of engaging and disengaging each connector 240 of the head strap 200 to and from the nasal cannula interface 1000 will now be described. Each connector 240 comprises a clip 241 having an elongate connector body 242 and a lateral projection 243 at a terminal end of the body 242. The lateral projection 243 comprises an inwardly facing engagement surface 243a. The face 244 of the connector 240 opposing the face 245 from which the projection 243 extends is preferably substantially smooth or planar. The corresponding formation 101/102 of the nasal cannula interface 1000 comprises a channel 101a/102a having entry 101b/102b and exit 101c/102c apertures at either end of the channel 101a/102a. A peripheral wall of the exit aperture 101c/102c defines an abutment 101ci/102ci configured to engage with the surface 243a of the projection 243 of the clip 241. A periphery 101bi/102bi of the entry aperture 101b/102b defines an abutment for engaging a flange 246 at an opposing end of the body 242 to the projection 243. This acts to limit the extent of insertion of the connector 240 into the corresponding channel 101a/102a. The flange 246 may be provided by a terminal end of the strap connection mechanism and/or the side arm 270.

Each section on either side of the head strap 200 and adjacent the respective primary end portion 201/202 includes or has applied thereto a side arm 270 comprising at least a face contacting surface for frictionally engaging with the patient's face to stabilise the headgear 200 on the face at the cheek, such as the cheekbone or below or a region thereof, both during coupling of the headgear to the nasal cannula interface 1000 and after when in use. The face contacting surface is preferably of a relatively higher frictional surface material than the remainder of the strap 200.

The side arm 270 may be a cheek support and/or a sleeve.

The face contacting surface is adapted to extend over a portion of the side of a patient's face in use, preferably at or at least substantially towards the patient's cheek, to assist with retaining or stabilising of the nasal cannula interface 1000 upon the face of a patient. The face contacting surface, being locatable at the cheek of the patient, further assists in keeping a remainder of the head strap 200 separated from and preferably extending below the eye or the orbit of the eye of the patient, so as to prevent obstruction of vision and/or discomfort resulting from the head strap 200 bridging at or near the eye or eye orbit.

It will be appreciated the face contacting surface may be adapted to extend over a portion of the side of a patient's face in use, for example, extending from at or near or above the left and right outer upper lips rearwardly and upwardly across the left and right cheeks.

The frictional surface material may be provided in the form of an elongate side arm 270 that is configured to receive the respective primary end portion 201/202 of the strap 200. The side arm 270 is configured to removably couple (or alternatively be permanently coupled) about the strap 200, a section of the strap 200 and/or a cannula connector 240/260 at the primary end portion of the strap.

The side arm (as for example being a sleeve) 270 is coupled about the strap 210 at the primary end portion 201/202 and also about a portion of the connector 240. The strap 210 extends through a passage 272 in the side arm 270, as can be seen in FIG. 7B. The strap 210 is adapted to be threaded through this passage and preferably remains free to be stretched or elasticised or extended when in a sleeved configuration. The connector 240 is substantially housed by the side arm 270 or shrouded by the face contacting surface to minimise direct contact with the patient's skin thereby improving stability comfort of the headgear 200. The clip 241 extends from an end 273 of the side arm 270. In another embodiment, the side arm 270 can be over-moulded on the connector 240 and/or the strap 210.

Referring to FIGS. 8A and 8B, the side arm 270 may be coupled about the connector 260 extending from the strap 210 at the primary end portion 201/202. In this embodiment the connector 260 is substantially housed by the side arm 270 or shrouded by the face to minimise direct contact with the patient's skin thereby improving stability and comfort of the headgear 200. In other words, the connector 260 extends fully though the passage 272 of the side arm 270. The buckle 251/252 extends from an end 274 of the side arm 270 and the clip 261 extends from the opposing end 273.

The side arm 270 may be pre-formed to have a curved shape along its length, such as one with an angle between flat and 20 degrees for example. The curve allows the side arm 270 to fit the contour of the patient's face or cheek in the region of the sleeve in use. Alternatively, the side arm 270 may elastically or non-elastically deform to take on the shape of a curved sleeve upon engagement with the primary end portion 201/202 or connector 260 of the head strap 200.

The side arm 270 provides the face contacting surface of relatively higher frictional surface material for frictionally engaging with the patient's face or facial skin. This face contacting surface is to be positioned for frictional engagement with the facial cheek skin of a patient. The face contacting surface is at least localised to the strap or the section of strap which is to be positioned upon the cheeks of a patient. The face contacting surface provided with the relatively higher frictional surface material is preferably of a material that is smooth and comfortable on the skin of the patient. The side arm 270 or at least the face contacting surface is therefore formed from a relatively softer material than the connectors 240 and 260.

In one preferred embodiment, the face contacting surface or the side arm 270 is formed from a soft thermoplastic elastomer (TPE) but may alternatively be formed from another plastics material such as silicone, or any other biocompatible materials.

Headgear for other forms of patient interfaces in addition to nasal cannula may comprise side arm 270 as cheek supports as described or similar, at or adjacent either side end of straps of headgear of the patient interface, which connect to the mask, for frictionally engaging with the patient's face to stabilise the mask on the face at the cheeks, and particularly for example direct nasal masks comprising nozzles or pillows which enter or engage the nares of the wearer. Such headgear may again comprise a single head strap adapted to extend in use along the patient's cheeks, above the ears and about the back of the head, with ends comprising clips in any suitable form which couple to the mask on either side (or are permanently attached to the mask).

The patient sensor 29, for example in the form of a pulse oximeter sensor, may be provided on the nasal cannula interface 1000 of FIGS. 7 to 9.

The patient sensor 29 may be provided on nasal cannula interface 1000 in accordance with any of the configurations described with reference to the nasal cannula 100 of FIGS. 1 to 6B.

The patient sensor 29 may be provided on the headgear 200, or on another removable part of the nasal cannula interface 1000 that connects to the main body 110, or the gases flow part 120 of the nasal cannula interface 1000. In this way, if the main body 110, and/or the gases flow part 120 are replaced or disposed of, the patient sensor 29 can be retained with the headgear 200 or other removable part, so that the patient sensor 29 is not disposed of and can be reused. For example, the patient sensor 29 could be provided on headgear 200 which is configured to connect to multiple different sizes of main body 110, and/or gases flow part 120. This allows a user to swap or replace parts of the cannula without having to dispose of the patient sensor 29.

Referring to FIGS. 7A to 9C, the patient sensor 29 may be provided on side arm 270. Any wiring associated with patient sensor 29 may extend through passage 272 of side arm 270 and extend from the end 274.

The patient sensor 29 may be recessed into the face contacting surface of the side arm 270 and may be flush with that face contacting surface. The patient sensor 29 may be located in any suitable position along the length of side arm 270, for example adjacent the formations 101, 102, or adjacent headgear strap 210.

The patient sensor 29 may be permanently mounted on the side arm, for example the patient sensor 29 may be overmolded onto the side arm 270.

The patient sensor 29 may be removably mounted on the side arm 270 so that the patient sensor 29 can be replaced or reused if the side arm 270 is disposed of. The patient sensor 29 can be removed, wiped, and incorporated into a different cannula having a similar recess in the side arm 270 to receive the patient sensor 29. This allows the sensor to be reused for patient's thereby reducing costs to a medical care facility.

Alternatively, the patient sensor 29 may be provided in a complimentary sensor body that can be permanently or removably mounted on the side arm 270, for example in a corresponding recess on the side arm 270. The recess and complimentary body may be provided with one or more retaining formations configured to retain the body in the recess. The patient sensor 29 being incorporated into the side arm 270 provide the patient sensor 29 in contact with the cheek region, e.g., the buccal or temporal regions of the face. There are blood vessels in this area of the face that the sensor can be positioned adjacent and used to detect the blood oxygen saturation of the patient.

A patient interface, such as a nasal cannula interface 1000 in accordance with any of FIGS. 7A to 9C, may comprise a plurality of patient sensors 29.

For example, the patient interface may comprise multiple patient sensors 29 (i.e., multiple pulse oximeter sensors) incorporated into the patient interface. For example, each or at least one side arm 270 may have one or a plurality of pulse oximeter sensors 29 that are positioned on or in the side arm 270 (i.e., sleeve). The measurements from these multiple sensors 29 can be averaged by the controller to provide a blood oxygen (SpO₂) reading.

Each side arm 270 may therefore comprise a single patient sensor 29.

In a further alternative form, each side arm 270 (i.e., each side arm) of the cannula may comprise multiple patient sensors. One, some or all of the plurality of patient sensors may be removable. Each side arm 270 may comprise a plurality of recesses or openings to receive patient sensors 29.

Multiple patient sensors 29 can be advantageous, as averaging the measured values can provide for a more accurate SpO₂ reading and reduce noise in the sensor readings received by the controller.

As described in relation to the arrangement of FIGS. 7A to 8B, a patient sensor may be retained within a component of the patient interface, namely a side arm 270. A side arm 270 which accommodates one or more patient sensors may be provided as part of a patient interface kit, or may be provided as a separate part which a user can switch out for a conventional side arm of a patient interface.

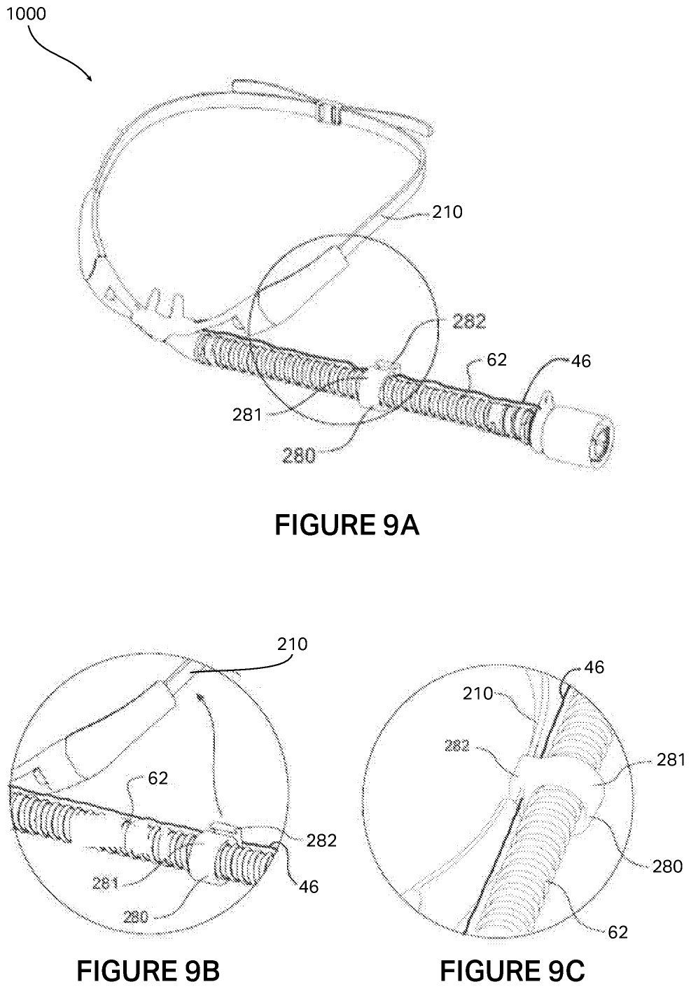

Referring to FIGS. 9A to 9C, a retention clip 280 may be provided that comprises a tubular body 281 for receiving and accommodating a portion of the gases inlet conduit 62 therein. A hook 282 projects from the tubular body 281 to couple the strap or other component of the headgear 200. In this manner the gases inlet conduit 62 can be coupled or tethered to the head strap 210 or headgear 200 in use. If the gases inlet conduit 62 is pulled, the force will be exerted onto the head strap 210 and not directly on the cannula 100. This relocation of force will reduce the likelihood of the nasal prongs 111 and 112 of the cannula 100 flicking out of the patient's nostrils.

One or more tethering points for connecting the clip 280 may be available on the headgear 200, with preferably at least two symmetric tethering points on either side of the headgear to increase usability.

It will also be appreciated the retention clip 280 may be removeable from or may be a permanent fitting on the gases inlet conduit 62.

The retention clip 280 may be connected or retained to a part of the patient interface, such as for example a part of a patient interface which provides for a relatively more rigid region (such as to facilitate support of the gases inlet conduit 62).

The retention clip 280 may also be positioned or affixed at a particular location on the gases inlet conduit 62, for example a predetermined location may be provided which holds the retention clip 280 in place.

The retention clip 280 may be configured to retain the wiring of the patient sensor 29, to secure the wiring against the gases inlet conduit 62. The patient sensor wiring can therefore extend in parallel with the longitudinal axis of the gases inlet conduit 62.

The gases inlet conduit 62 may be provided with one or more sensor wires, for example in the wall of the conduit, or extending through the bore of the conduit. The one or more sensor wires may be configured to be electrically coupled to the patient sensor 29.

Such electrical coupling could be provided by a physical electrical coupling, such as via an electrical connector, between wiring of the patient sensor 29 and the one or more sensor wires in the gases inlet conduit 62.

Such electrical coupling could be provided via an inductive coupling. For example, patient sensor wiring may extend alongside arm 270 and/or may be provided in the main body 110, and/or the gases flow part 120 of the nasal cannula interface 1000. Conduit wiring may extend to a position at or adjacent an end of the gases inlet conduit 62, where the gases inlet conduit 62 is connected to the inlet of the nasal cannula. The gases inlet conduit 62 and the nasal cannula interface 1000 may be provided with inductive couplers configured to electrical couple the gases inlet conduit 62 to the patient sensor wiring.

Such an arrangement would remove or reduce the need for a physical electrical connector or the like, and the need for one or more exposed electrical contacts. Such an arrangement would also remove or reduce the number of connections needing to be made by a user in use of the nasal cannula interface 1000. For example, if the patient sensor 29 and headgear 200 were to be reused, the user would not have to physically disconnect the patient sensor wiring from the gases inlet conduit 62.

The gases inlet conduit 62 may be a heated or an unheated conduit. The conduit may be an extension of any desired length.

As described above, the patient sensor 29 may be configured to contact the face of a patient.

The patient sensor 29 may be an optional addition to the patient interface. As an addition to a patient interface, a patient sensor may be incorporated with a patient interface by way of an accessory. The accessory is configured to retain a sensor for measuring at least one patient parameter. The accessory is configured to attach to the patient interface.

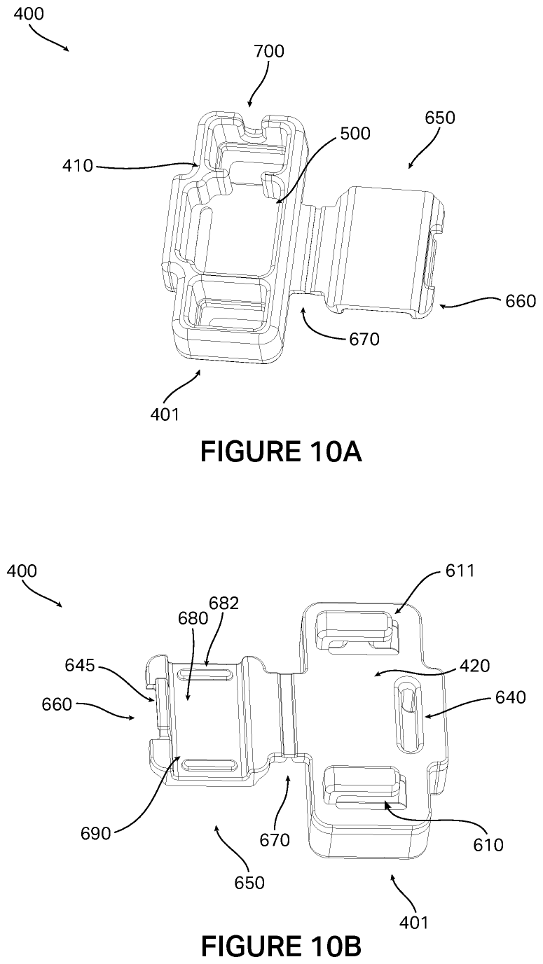

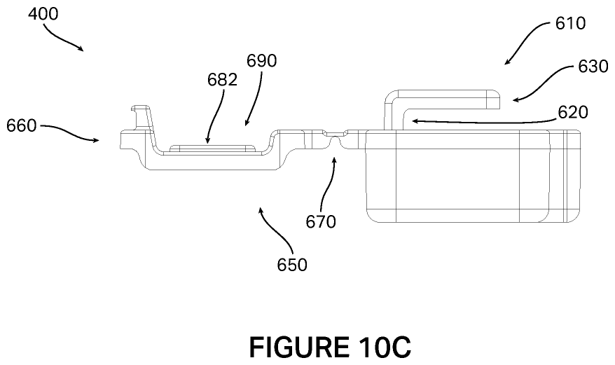

An accessory 400 may be provided as a nasal cannula interface accessory, as shown for example in FIGS. 10-16.

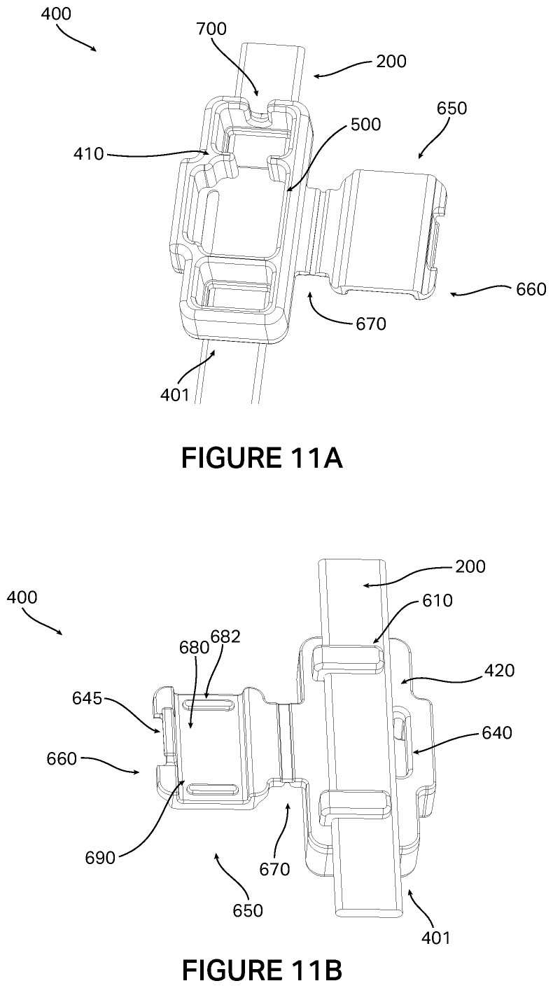

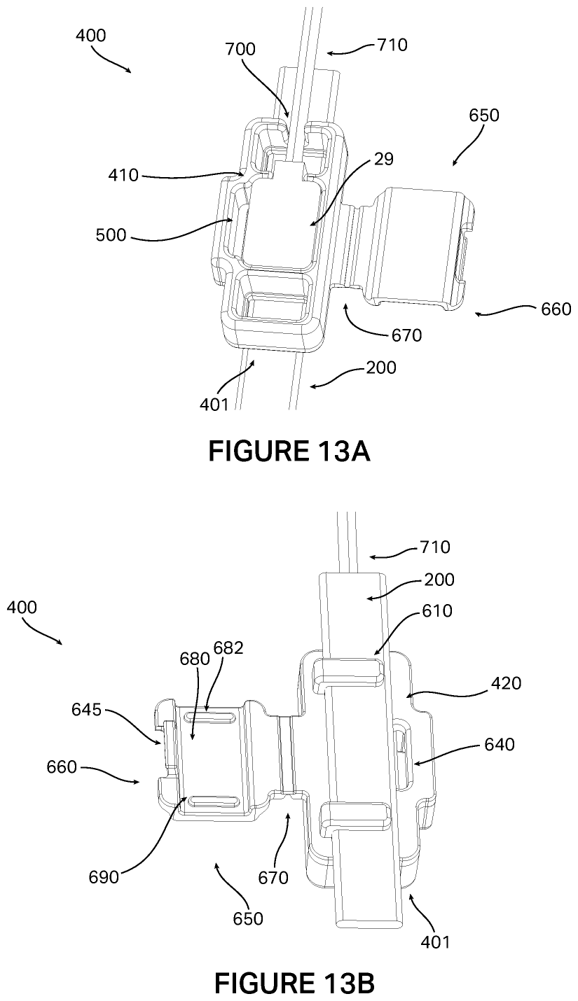

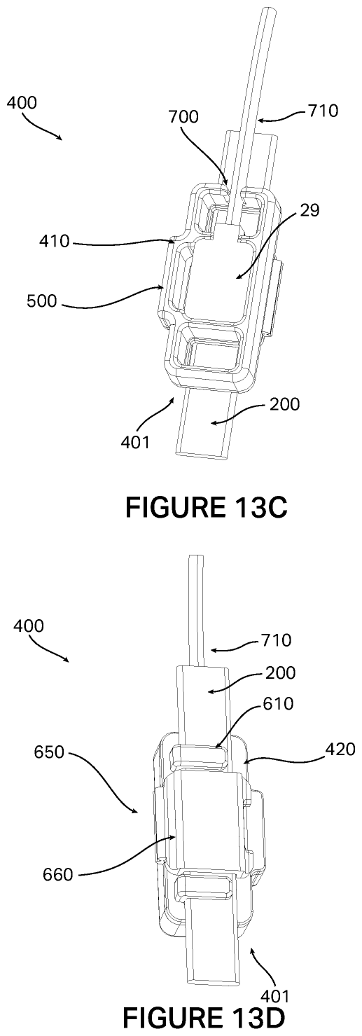

The accessory 400 may be configured to attach to a nasal cannula interface 1000. For example, it may be connected to the strap 200 of the nasal cannula interface 1000 as shown in FIGS. 11, 13,15F and 15D.

It will be appreciated that the accessory 400 could be used on any suitable patient interface with a strap (and not limited to a nasal cannula interface). However, as an example patient interface, a nasal cannula interface is used below.

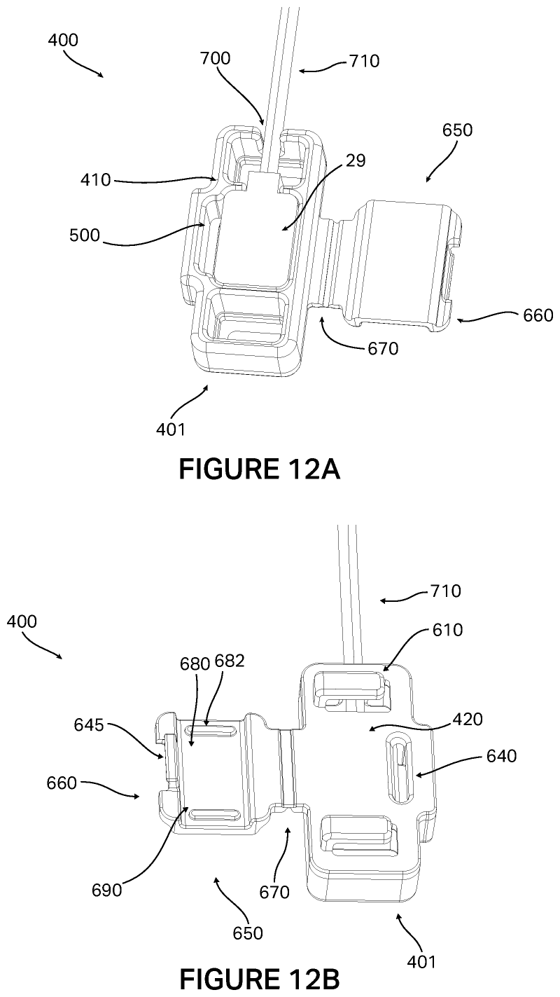

As shown in FIG. 12A, the accessory 400 comprises a sensor cavity 500, at least one securement feature 610, 650, and a wire cavity 700. The sensor cavity 500 is configured to retain a patient sensor 29, as shown in the view of FIG. 12A. As described in more detail above, the patient sensor 29 may be configured to measure at least one patient parameter. The at least one securement feature 610, 650 is configured to connect the accessory 400 to the nasal cannula interface 1000; for example, the at least one securement feature 610, 650 may be releasably connected to the strap 200 of the nasal cannula interface 1000 as shown in FIGS. 11A and 11B, and 13A and 13B.

The accessory 400 may comprise a main body 401.

The sensor cavity 500 and/or wire cavity 700 may be provided in a main body 401 of the accessory 400.

The securement feature 610, 650 may also extend from the main body 401.

The at least one securement feature 610, 650 may be configured to releasably connect the accessory 400 to the nasal cannula interface 1000. In some configurations, the at least one securement feature 610, 650 may be configured to permanently connect the accessory 400 to the nasal cannula interface 1000 so as not to be removeable (however optionally still moveable along the strap as described in more detail below).

The wire cavity 700 may be configured to provide a pathway to the sensor cavity 500 for one or more wires 710, as shown in FIG. 12A.

The accessory 400 may have one or more gripping features. The gripping features may comprise, for example, protrusions which may aid or improve the gripping of the accessory 400 to the nasal cannula interface 1000. The gripping features may help the user move the accessory 400 relative to the nasal cannula interface 1000 to position the accessory 400 (as described in more detail below).

As shown in FIGS. 15E and 15F, a nasal cannula interface 1000 may be provided. The nasal cannula interface 1000 may comprise a nasal cannula interface accessory 400.

The accessory 400 may be releasably connected to the interface 1000.

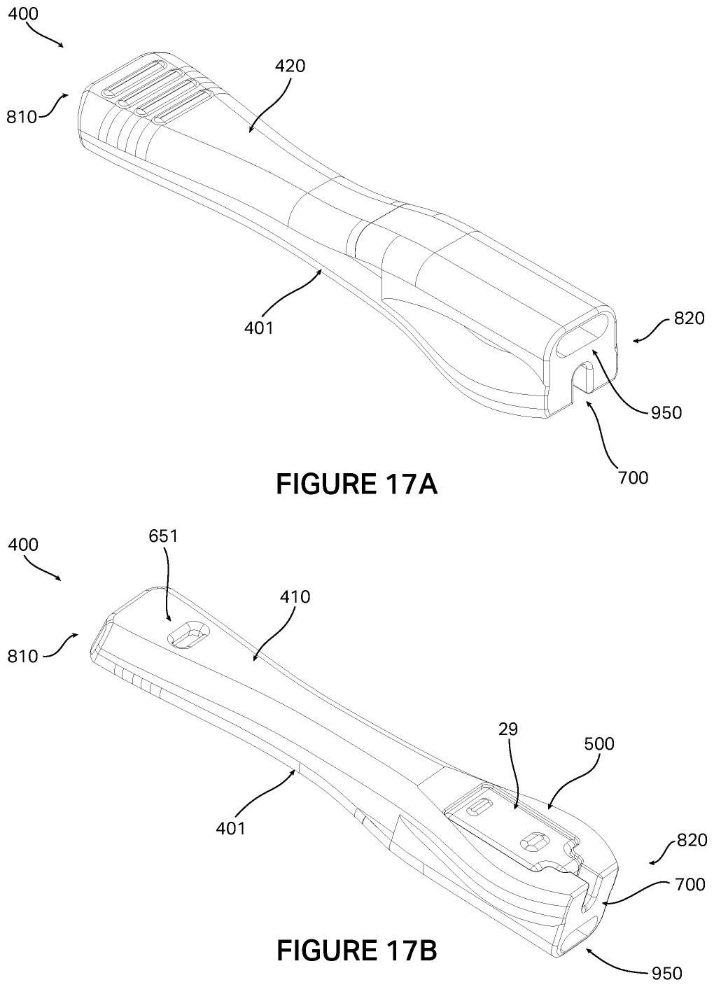

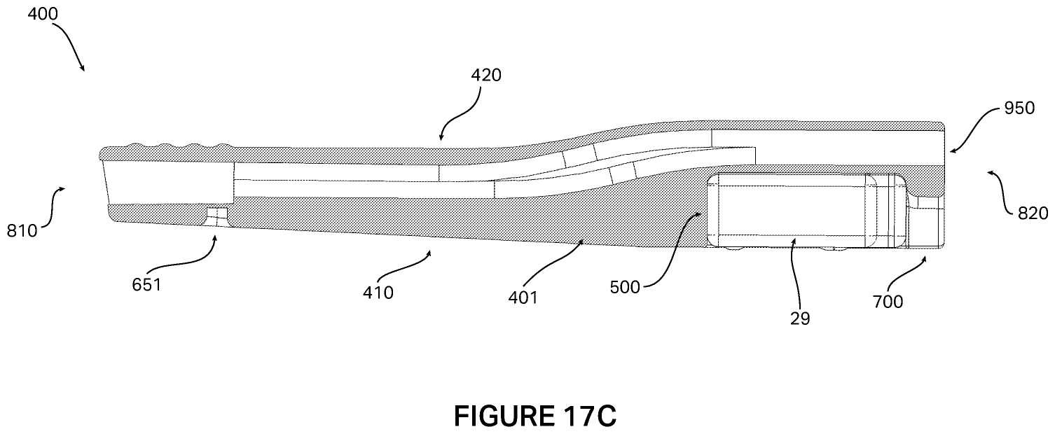

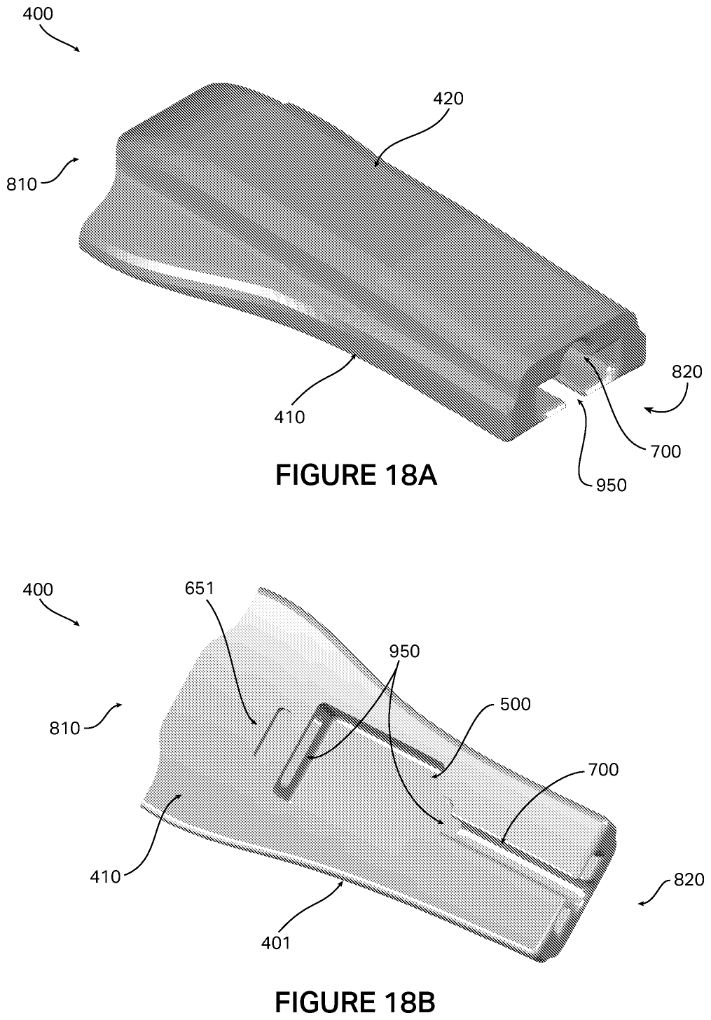

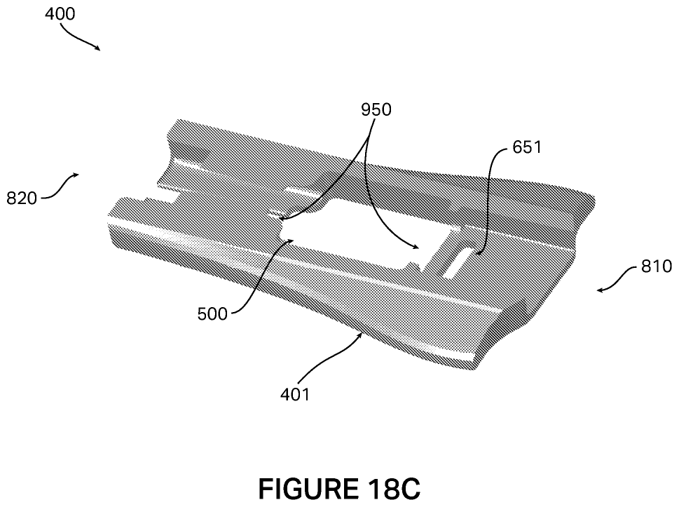

The accessory 400 may replace a component of the interface 1000, for example, as shown in FIGS. 17A-18C, the accessory replaces the side arm (as a cheek support). In this case, the interface may be provided without a side arm.

As described in more detail below a component may be provided which replaces a component of the interface.

The example of the accessory in FIGS. 17A-C and 18A-C is a side arm 270 which, as shown in FIGS. 7A-8B, and may be part of a nasal cannula.

As described above, the side arm 270 may be a check support configured to rest, at least in part, on a patient's cheek in use.

As shown in FIGS. 17A-18C, the accessory 400 comprises a sensor cavity 500, a wire cavity 700, and a strap passage 950.

In some configurations, the accessory 400 may comprise a securement feature. The securement feature may be located at, or near, a first end 810 of the accessory 400.

The securement feature may be configured to connect to the cannula connectors 240 as described above in relation to FIGS. 7D to 7H.

In some embodiments, the securement feature is connected to the strap 210 (as with the embodiments of FIGS. 10A-16E).

As shown in FIGS. 17A-18C, the sensor cavity 500 may be configured to retain a patient sensor 29 (as shown in FIGS. 17B and 17C) configured to measure at least one patient parameter. The wire cavity 700 may be configured to provide a pathway to the sensor cavity 500 for one or more wires 710.

The accessory 400 may comprise a main body 401.

The sensor cavity 500 and/or wire cavity 700 may be provided in a main body 401 of the accessory 400.

In some configurations, the accessory 400 may have a securement feature configured to connect to a cannula connector 240.

In some configurations, for example, as shown in FIGS. 17A-18C, the accessory 400 comprises a strap passage 950. The strap passage 950 may extend from a second end 820 of the accessory 400 along, at least a portion of a longitudinal axis of the accessory 400.

The strap passage 950 may allow for passage of the strap through the accessory 400 to the main body 110 (or another component of the patient interface).

The strap passage 950 may allow for passage of the strap through the accessory 400 to a main body connection feature configured to connect the strap of the nasal cannula interface 1000 to a main body 120. The connection feature may be the cannula connectors 240 as described above in relation to FIGS. 7D to 7H.

The strap passage 950 may be located in the main body 401 of the accessory 400.

The strap passage 950 may be configured to contain the strap 200 of the nasal cannula interface 1000.

The strap passage 950 may be shaped to contain the strap 200. That is, the strap passage 950 may have a shape that corresponds to the shape of the strap 200. For example, as shown in FIGS. 17C, 18B and 18C, the strap passage 950 may comprise a rectangular cross section to correspond to a substantially flat, rectangular strap 200. In some configurations, the strap passage 950 may have a circular and/or oval cross section.

In some configurations, the strap passage 950 may have an opening at a first end of the accessory 400, and an opening at the second end of the accessory 400, and a portion in between each end that is enclosed. In some configurations, the strap passage 950 may be at least partially exposed to an external face of the accessory 400. For instance, in FIGS. 18A-C, and in particular in FIG. 18B, at least a portion of the strap passage 950 is exposed to an external face of the accessory 400.

In some configurations, the strap passage 950 may be separate to the sensor cavity 500, for instance, in FIG. 17C. In some configurations the strap passage 950 may be part of to the sensor cavity 500. For example, as shown for example in FIG. 18B and the cross-sectional FIG. 18C, the strap passage 950 is located, at least partially, in the sensor cavity 500.

In some configurations, the strap 200 may be adhered or otherwise connected to at least one surface of the strap passage 950. For instance, the strap 200 may be connected to the at least one surface of the strap passage 950 by an adhesive; or by way of further example, the strap 200 may be overmolded within the strap passage 950. In some configurations, the strap 200 may be physically retained by another part of the accessory 400 for example the strap 200 may not be adhered to, or otherwise attached to, the at least one surface of the strap passage 950.

It will be appreciated that, where appropriate, the disclosure below in relation to the sensor cavity 500 may be applied to both the accessory 400 and component 2000 (as described in more detail below).

As shown in FIGS. 10A, 11A, 12A, 13A, and 16B the sensor cavity 500 is formed on a first face 410 of the nasal cannula interface accessory 400, as shown in FIGS. 10A, 11A, 12A, 13A, and 16B.

The sensor cavity 500 may be formed within a main body 401 of the accessory 400.

As shown in FIGS. 17B, 18B, and 18C, the sensor cavity 500 is formed on a first face 410 of the accessory 400, as shown in FIGS. 17B, 18B, and 18C.

The sensor cavity 500 may be formed within a main body 401 of the accessory 400. As shown in FIG. 17C, the sensor cavity 500 may be a substantially enclosed internal space of the main body 401 of the accessory 400. In other configurations the sensor cavity may extend at least in part to an external face of the accessory.

The sensor cavity 500 may be shaped to receive the sensor. In some configurations the sensor cavity 500 is: square, rectangular and/or circular. The sensor cavity 500 may have substantially rounded edges and vertices. The rounded edges and vertices may assist positioning of the sensor in the sensor cavity 500 and prevent damage to the sensor. In an example as shown in FIGS. 10A, 11A, and 16B the sensor cavity 500 may be rectangular with substantially rounded edges and vertices.

The sensor cavity 500 may be a friction fit with the patient sensor 29 (for example the housing of the sensor). In some configurations, an adhesive may be used to retain the patient sensor 29 in the sensor cavity 500.

The patient sensor 29, as described in more detail above, may be a patient sensor configured to measure a patient parameter. The patient parameter may be a physiological parameter. For instance, the patient parameter may by a measure or indication of blood oxygenation of the patient.

The sensor cavity 500 may be arranged to orient the patient sensor 29 to be in contact with a patient. As shown in FIGS. 10A, 11A, 12A, 13A, 16B, 17B, 18B and 18C, the sensor cavity 500 comprises an opening on a first face 410 of the accessory 400 which is configured to face a patient in use. For example where the patient sensor 29 is a pulse oximeter sensor, the patient sensor 29 may comprise a transducer (not shown), and the sensor cavity 500 may be arranged to orient the sensor such that the transducer of the patient sensor 29 faces towards the patient (such that the transducer contacts skin or position adjacent skin). In some configurations, the transducer of the patient sensor may comprise a light transducer. The light transducer may be an infrared transducer and/or a red light transducer. The light transducer may be a photodiode or phototransistor.

In some configurations, the patient sensor comprises at least one light source. The source being configured to be directed towards a patient's skin. The light source may be an infrared source and/or a red-light source. The light source may comprise an LED. In some configurations, the light source may comprise a plurality of light sources, each light source emitting light at a different wavelength. For example, the light source may comprise at least one red LED and at least one infrared LED. Each light source may have a corresponding transducer.

The wire cavity 700 may be formed on the same face, or a different face as the sensor cavity 500 of the accessory 400. The sensor cavity 500 may be formed on the first face 410 of the accessory 400, and the wire cavity 700 may be located on the second face (for example adjacent the first face) 420 of the accessory 400. By way of further example, as shown in FIGS. 12A, 13A and 16B, the sensor cavity 500 and the wire cavity 700 may both be formed on the first face 410 of the accessory; or, as shown in FIGS. 17B, 18B, and 18C, the sensor cavity 500 and the wire cavity 700 may both be formed on the first face 410 of the accessory.

The wire cavity 700 may be formed adjacent to the sensor cavity 500, as is shown by FIGS. 12A, 13A, 16B, 17B, 18B, and 18C.

The wire cavity 700 may extend from an external face to the sensor cavity 500. The wire cavity 700 may extend from a face of the accessory 400 to the sensor cavity 500.

The wire cavity 700 may comprise an opening on a face of the accessory 400 which is configured to face a patient in use. As shown in FIGS. 18A and 18B, the wire cavity 700 may comprise an opening on the first face 410 of the accessory 400 so as to face the patient in use—this may aid in assembly of the patient sensor 29 and sensor cavity 500 by allowing the wire to be easily inserted into the wire cavity 700. By way of further example, as shown in FIGS. 10A, 11A, 12A, 13A, and 16B, the wire cavity 700 may comprise an opening 651 on the first face 410 of the accessory 400 so as to face a patient.

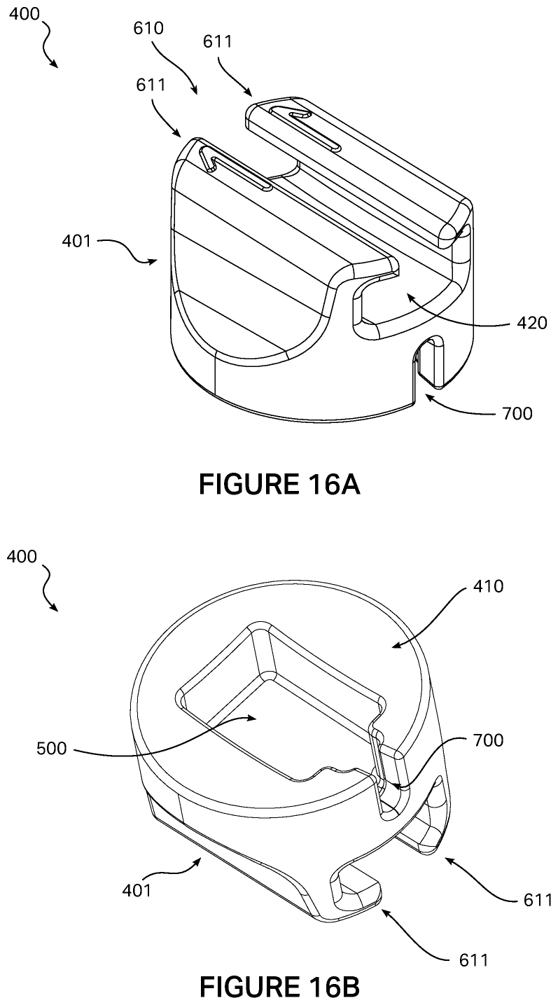

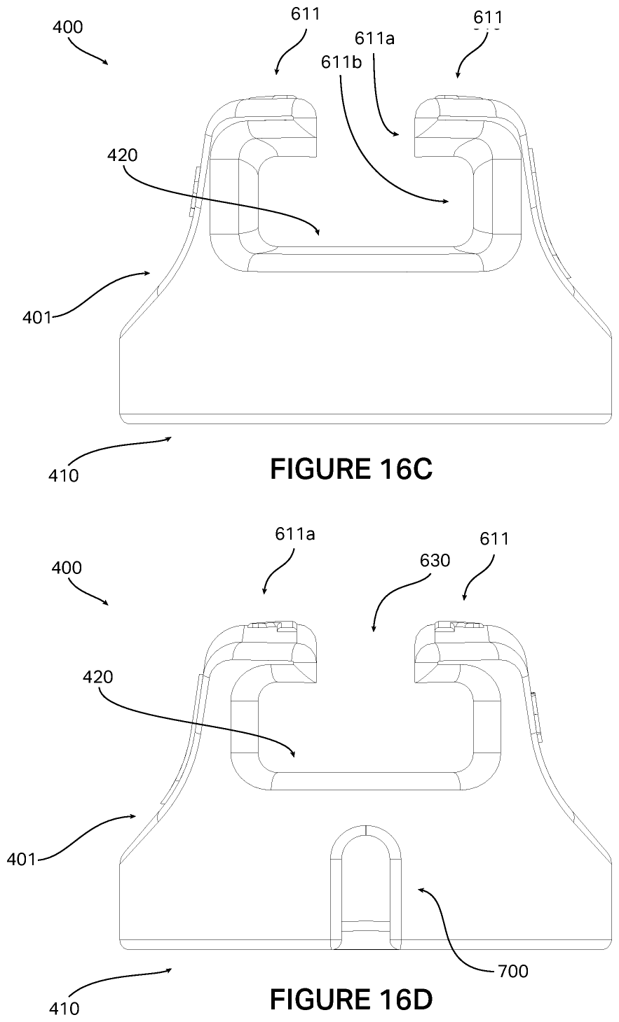

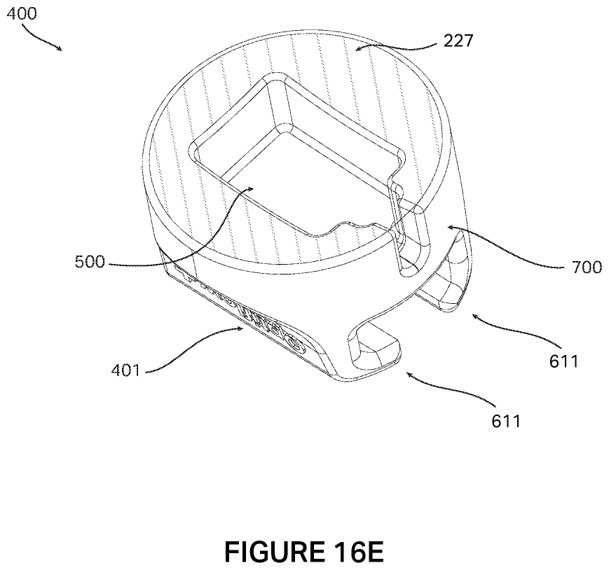

In some configurations, the wire cavity 700 may comprise a plurality of openings on different faces of the accessory (optionally adjacent faces) for example as shown in FIG. 16B where the wire cavity 700 has an opening on a side surface and an opening on a patient surface. This allows the wire to enter on a different face of the accessory to the face which contacts the patient in use.

The wire cavity 700, may extend through a single face of the accessory to the sensor cavity 500.

The wire cavity 700 may comprise a slot. Further, the wire cavity may be one or more of: square, rectangular and/or circular. For instance, as shown in FIGS. 12A, 13A, 16B, 17A, 18B, and 18C the wire cavity is substantially rectangular.

The wire cavity 700 may, for example, be a groove and/or cut-out of the accessory (for example, the main body of the accessory). In some configurations, the wire cavity may extend from a side face, or a rear face of the accessory 400.

In some configurations, the wire cavity 700 may comprise one or more retention features. The retention features may aid in retaining the wire in the wire cavity 700 once installed. The retention features may comprise at least one ledge extending from a perimeter of the wire cavity 700. The at least one ledge may extend from alternating sides of the wire cavity (so as to not overlap).

The wires 710 that the wire cavity 700 is configured to provide a pathway for may comprise a cable, a cord, a lead, cable harness or any other insulated assembly of electrically conductive material. In some configurations, a single wire 710 may be accommodated in the wire cavity 700. In other configurations, more than one of the same type or different types of wires 710 may be accommodated in the wire cavity 700.

In some configurations, the wire cavity 700 may, for example, be an aperture allowing for a pathway to the sensor cavity 500 for one or more wires 710.

The accessory 400 may be attached to the nasal cannula interface 1000 through the at least one securement feature 610, 650. The at least one securement feature 610, 650 may be configured to retain the accessory 400 to a strap 200 of the nasal cannula interface 1000.

The at least one securement feature 610, 650 may be configured to extend from a side (for example, a face) of the accessory 400 opposite the sensor cavity 500. As shown by FIGS. 10B, 11B, 12B, 13B and 16A-E, the at least one securement feature 610, 650 may be configured to extend from a second face 420 of the accessory 400, the second face 420 being provided on the opposite side to the first face 410.

The securement feature 610, 650 may retain the accessory 400, whilst allowing for the user to move the accessory 400 relative to the nasal cannula interface 1000 and/or remove the accessory 400 from the nasal cannula interface 1000. The securement feature 610, 650 may be configured to both substantially prevent movement of the accessory 400 along the strap 200 and allow relative movement of the strap 200 and accessory 400. The securement feature 610, 650 may be configured to allow relative movement of the strap 200 and accessory 400 (for example, by allowing the accessory 400 to move relative to the strap 200) when a threshold force is applied. By configuring the accessory 400 to be moveable along the length of the strap 200, the clinician may be able to position the patient sensor 29 in a desired location. The required type and degree of threshold force may depend on the type of securement feature 610, 650 and the material of the strap 200. It will be appreciated that the securement features 610, 650 may comprise one or more features, for example: a fastener (i.e., hook and loop-comprising a plurality of hooks and a plurality of loops which are configured to engage with each other; the hook and or loop may be provided on a substrate), magnets, one or more hooks, and/or geometric features.

The securement feature may allow for the accessory 400 to be connected and disconnected from the strap 200 so as to be relocated along the length of the strap 200 (as described in more detail below).

Referring now to FIGS. 16A-E, the at least one securement feature 610, 650 may comprise at least one arm 611. The at least one arm 611 may be configured to extend around at least part of the strap 200 of the nasal cannula interface 1000.

The at least one arm 611 may comprise a first portion 611a and a second portion 611b. The first portion 611a may extend from the accessory 400: optionally, the first portion 611a may extend in a perpendicular direction from the second face 420 of the accessory 400. The second portion 611b may be configured to be oriented substantially perpendicular to the first portion 611a, and/or parallel to the accessory 400 and/or the second face 420 of the accessory 400.

The at least one securement feature 610, 650 may comprise a pair of arms 611, as shown in the configuration depicted by FIGS. 16A-16D. The pair of arms 611 may be configured to releasably connect the accessory 400 to the nasal cannula interface 1000 and/or optionally to the strap 200 of the nasal cannula interface 1000.

As shown in FIGS. 16A-E, the pair of arms 611 may extend from the accessory 400, and each arm 611 may extend towards the other arm and/or towards a center of the accessory 400. The arms 611 may be arranged along an axis of the accessory; the axis may, for example, be configured to be parallel to an axis of the strap 200 when the accessory 400 is connected to the strap 200.

Each arm 611 of the pair of arms 611 may comprise a first portion 611a and a second portion 611b. The first portion 611a of each arm 611 may extend from the accessory 400: optionally, the first portion 611b may extend in a perpendicular direction to the second face 420 of the accessory 400. The second portions 611b of each arm may be configured to be oriented toward each other and/or the second portions 611b may be configured to be oriented toward a center of the accessory. Optionally, the second portion 611b of each arm 611 may be oriented substantially perpendicular to the associated first portion 611a, and/or parallel to the accessory 400 and/or the second face 420 of the accessory 400. In some configurations, the first portion 611a may extend vertically upwards from the accessory 400, and the second portion 611b may extend inwards towards a center of the accessory 400. In some configurations, the first portion 611a may extend vertically upwards from the accessory 400, and the second portion 611b may extend towards the second portion 611b of the other arm 611. The first portion 611a and the second portion 611b of each arm 611 may form an angle that is less than 90 degrees, or about 90 degrees, or less than about 120 degrees.

Each arm 611 may form a ‘U’ shape with the second face 420 of the accessory. The U shape formed by one arm may face the U shape formed by the other arm so as to retain the strap of the nasal cannula interface.

The at least one arms 611 may be configured to receive the strap 200 of the nasal cannula interface 1000. For instance, in configurations where the securement feature 610, 650 comprises a pair of arms 611, a gap 630 may be defined between the pair of arms 611. The strap 200 of the nasal cannula interface 1000 may be configured to be insertable to the gap 630 to be connected to the accessory 400. The pair of arms 611 of the securement feature 610, 650 may be configured to receive the strap 200 of the nasal cannula interface 1000 to be connected to the nasal cannula interface 1000. The width of the strap 200 of the nasal cannula interface 1000 may be larger than the gap 630. The strap 200 may only be inserted into, and/or removed from, the gap 630/the accessory 400 when aligned with an edge of the strap 200 or when the strap 200 is folded along the width of the strap 200.

The at least one arms 611 may comprise a plurality of arms.

Referring now to FIGS. 10A-15F, the at least one securement feature 610, 650 may comprise a clip 650.

The clip 650 may be connected to the accessory 400; it may be configured to secure the accessory 400 to the nasal cannula interface 1000, for example, it may secure the accessory 400 to the strap 200 of the nasal cannula interface 1000.

The clip 650 may extend from the accessory 400, in some configurations, the clip 650 may extend from the second face 420 of the accessory 400 as shown in FIGS. 10A-15F.

The clip 650 may comprise a clip arm 660. The clip arm 660 may be connected to the accessory 400 through a biasing element 670. The biasing element 670 may comprise a hinge or spring or a geometric feature. The biasing element 670 may bias the clip arm 660 to a closed position or an open position. FIGS. 10A-15F show the exemplary configuration of a clip arm 660 comprising a hinge 670 in open position.

As shown in FIGS. 10A-15B the hinge 670 is a living hinge.

The clip 650 may be configured to retain the strap 200 of the nasal cannula interface 1000 to the accessory 400 when it is in a closed position. When the clip 650 is in a closed position (as, for example, shown in FIGS. 13D and 15D), the clip may retain the strap 200 between the clip 650 and the nasal cannula interface 1000 to the accessory 400. When the clip 650 is closed and retaining the strap 200 of the nasal cannula interface 1000, the accessory 400 may be prevented from sliding relative to the strap 200.

The clip arm 660 may comprise a contacting surface 680 as shown by FIGS. 10B, 11B, 12B 13B, 14B, and 15B. The contacting surface 680 may be configured to engage with the strap 200 of the nasal cannula interface 1000 when the clip 650 is in a closed position.

The contacting surface 680 may be located within a recess 690 of the clip arm 660, as shown in FIGS. 10B, 11B, 12B 13B, 14B, and 15B. The recess 690 may be shaped to receive the strap 200 of the nasal cannula interface 1000.

The contacting surface 680 may comprise at least one protrusion. The at least one protrusion may be configured to aid in retaining the strap 200 when the clip 650 is in a closed position (for example, as shown in FIGS. 13C, 13D, 15C and 15D).

The at least one protrusion may comprise at least one rib 682. The at least one rib 682 may be located perpendicular to a width of the strap 200 when the strap 200 is engaged with the contacting surface 680, and/or perpendicular to a longitudinal axis of the contacting surface 680. In some configurations, as shown in FIGS. 10B, 11B, 12B, and 13B, the at least one rib 682 comprises a pair of ribs 682. The pair of ribs 682 may be located at opposing ends of the contacting surface. When the strap 200 is engaged with the contacting surface 680, the pair of ribs 682 may be spaced along a length of the strap 200, and/or a longitudinal axis of the contacting surface 680.

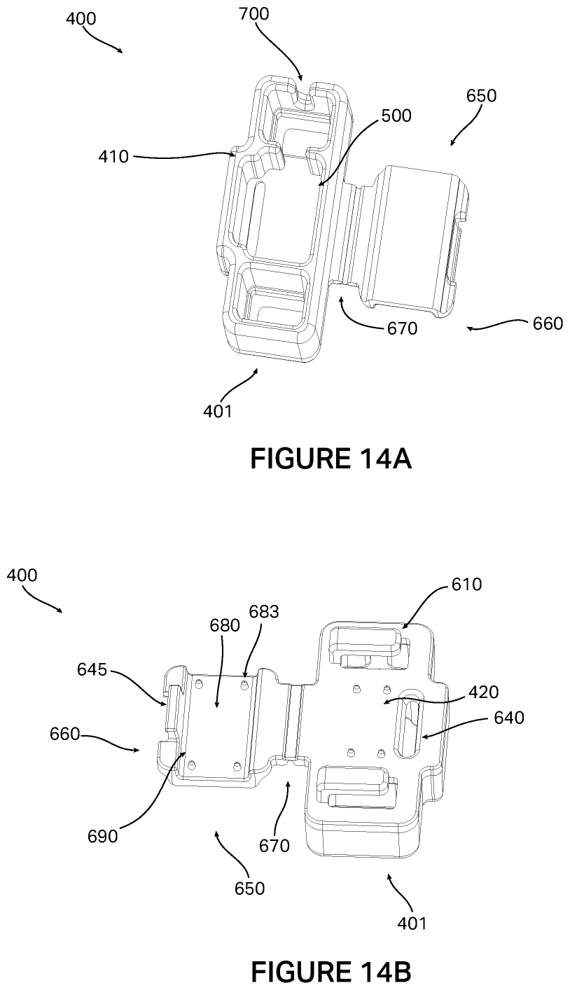

The at least one protrusion may comprise one or more bumps 683. The one or more bumps 683 may, for instance, comprise a bump 683 located in each corner of the contacting surface, as shown in FIG. 14B. By way of further example, as shown in FIG. 15B, the one or more bumps 683 may be patterned across the entire contacting surface 680, or at least a portion of the contacting surface 680. By way of yet another example, the one or more bumps 683 may be patterned in offset rows, such as is shown in FIG. 15B, or they may be patterned in aligned rows.

The contacting surface 680 may have a non-smooth and/or a substantially rough surface.

A surface which contacts the strap of the accessory 400 (for example, a rear face of the accessory 400) may comprise at least one protrusion. The at least one protrusion may be configured to aid in retaining the strap 200 when the clip 650 is in a closed position (for example, as shown in FIGS. 13C, 13D, 15C and 15D). The protrusion may be that as described above and may be configured to be complementary to the at least one protrusion of the contacting surface 680.

Referring now to FIGS. 15A and 15B, the clip arm 660 may comprise an aperture 695 that may extend through the clip arm 650, and optionally may extend through the contacting surface 680. The aperture 695 may be configured to receive a portion of another of the at least one securement system: optionally, the another of the at least one securement system may be at least one arm 610, for example, as shown in FIGS. 15A and 15B.

The accessory 400 may comprise a pair of arms 610 located on each side of the clip 650, for example, as shown in FIGS. 10A-14B (as described in more detail above). The second portions 630 of the arms are oriented in the same direction (and are optionally parallel).

This is in contrast to the arms 610 of FIGS. 16A-16B, where the second portions 630 extend towards each other.

The clip arm 660 may comprise at least one retention feature 640. The retention feature 640 may be configured to engage with a mating portion 645 of the accessory 400 configured to retain the clip 650 in a closed position. For example, as shown in FIGS. 10B, 11B, 12B, 13B, 14B, and 15B, the retention feature 640 comprises a protrusion, and the mating portion 645 comprises a corresponding recess. By way of further example, the retention feature 640 may comprise a recess, and the mating portion 645 may comprise a corresponding feature such as a protrusion.

The accessory 400 may be attachable to the nasal cannula interface 1000 at a plurality of interface attachment locations and/or a plurality of patient face locations. The plurality of patient face locations may correspond with the plurality of attachment locations.

The plurality of attachment locations on the nasal cannula interface 1000 may be defined by at least a connection point between the strap 200 and the main body 110 of the nasal cannula interface 1000.

The patient face locations may be locations where the accessory 400 is located in proximity to the patient's face. For instance, the patient face locations may be locations proximal to the patient's cheek, or by way of further example, may be locations proximal to the space between the patient's eye and lip.

The accessory 400 may be configured to be adjustable between a plurality of attachment locations. In some configurations the accessory 400 may be adjustable between the attachment locations without being removed from the nasal cannula interface 1000, or optionally, without being removed from the strap 200. For instance, the accessory 400 may be configured to be adjusted slidably between a plurality of attachment locations.

In some configurations the accessory 400 may be adjustable between the attachment locations by disconnection of the clip and the user moving the accessory to a new location before reengaging the clip (for example, by moving the clip to a close position).

The accessory 400 may be configured to be adjustable between the plurality of patient face locations. In some configurations the accessory 400 may be adjustable between the patient face locations without being removed from the nasal cannula interface 1000. For instance, the accessory 400 may be configured such that it may be adjusted slidably between the plurality of patient face locations (for example, in the direction along the strap as shown by the arrow in FIGS. 15E and 15F).

The accessory may be for instance located proximal to the patient's cheek. By way of further example, it may be located proximal to the space between the patient's eye and lip.

The accessory 400 being attachable to the nasal cannula interface 1000 at a plurality of locations along the strap 200 may allow for easier positioning of the patient sensor 29 relative to the patient. This allows for the position of the sensor to be modified based on, for example, the patient's facial structure, or the location of other medical devices (for example nasogastric tubes). For example, the accessory 400 may be moved to another side of the nasal cannula.

The accessory 400 being attachable to the nasal cannula interface 1000 at a plurality of locations along the strap 200 may also allow for a patient sensor 29 to be retrofitted to the nasal cannula interface 1000. This allows for a patient sensor 29 to be added to the nasal cannula interface 1000 on an as needed basis.

In some configurations, the accessory 400 may comprise an extension portion which extends away from the main body of the accessory 400. The extension portion may comprise the sensor cavity 500 and wire cavity 700 to allow the patient sensor 29 to be located at a specific location—for example, a patient's ear lobe.

As shown in FIGS. 16A-16C, the face of the accessory 400 which is configured to face a patient in use is substantially circular. The face of the accessory 400 which is configured to face a patient in use may be other shapes, for example, oval, or rectangular.

As shown in FIG. 16E, the face of the accessory 400 which is configured to face a patient in use may comprise at least one surface material 277. The at least one surface material 277 may cover a or the face of the accessory 400 which is configured to face a patient.

The surface material 277 may be located on, or be the face contacting surface, as described above.

The at least one surface material 277 may in some configurations (for example, as shown in FIG. 16E) not cover the patient sensor 29, and in some configurations, it may cover the patient sensor 29. The at least one surface material may be provided with an aperture such that the at least one surface material does not extend over the sensor cavity of the accessory.

The at least one surface material may be the same material as the accessory 400, or it may be a different material as the accessory 400. For example, the at least one surface material may be a fabric material and/or silicone and/or a thermoplastic elastomer, and the accessory 400 may be a plastic.

The at least one surface material and the accessory 400 may be, for instance, separable from the accessory 400 or integral with the accessory 400.

The at least one surface material may be a film or membrane.

The at least one surface material may be configured to increase friction between the accessory 400 and a patient's face. The at least one surface material may have adhesive or slip-resistant material properties. Further, the surface material may be configured to provide a frictional force to resist movement between the accessory 400 and the strap 200.

As described above a component may be provided to replace, or be a component of, the patient interface, for example the nasal cannula 100.

The component may be able to replace an existing component of the nasal cannula interface 1000. The component may be provided as an additional component which a user can optionally add to the patient interface (for example, as a replacement component), or as a component as part of patient interface which comes pre-assembled.

In some configurations, the component may be connectable as an intermediate component between other components of the nasal cannula interface 1000. For example, the component may be connectable between the side arm 270 and the main body 110.

The component may be connected to a strap. The component may be connected to the strap 210 at any point, but in some configurations, at a second end. The second end may be opposite the first end.

The component may comprise a connection feature.

The connection feature may be configured to connect to the nasal cannula interface 1000.

The component may be a side arm as described above.

The connection feature may be configured to connect directly to a main body of the nasal cannula interface 1000 (as a main body connection feature). In these embodiments, the main body connection feature may be or comprise features of the cannula connectors 240 as described above in relation to FIGS. 7D to 7H.

The component may be formed integrally (for example, by overmolding) with the connection feature and/or the strap. In some configurations, the strap 210 may be connected to the connection feature (for example, as described above in relation to FIGS. 7D to 7H) and then the component overmolded.

The connection feature may alternatively, or additionally, be a fastener (e.g., hook and loop-comprising a first substrate having a plurality of hooks and a second substrate comprising a plurality of loops), magnets, or geometric features.

The component may comprise any of the features of the accessory as described above.

In some configurations, the component may be the accessory of FIGS. 17A to 18C but permanently attached to the strap and/or cannula connector.

As previously described, as part of providing a patient with respiratory support, one or more physiological parameters of the patient may be measured by a patient sensor. Some patient sensors may require placement on or near the patient's body in order to measure the desired physiological parameter. Accordingly, as previously described, a patient respiratory interface may incorporate a patient sensor.

In various configurations that have been described, an accessory may be provided for a patient respiratory interface. The accessory is able to be attached to the patient respiratory interface. For example, the accessory may be connected to part of the headgear or may be attached to another part of the patient interface such as a main body of the patient interface itself. The accessory is configured such that one or more patient sensors may be mounted to the accessory. For example, an accessory maybe configured such that one or more patient sensors may be retained by the accessory. The accessory may include a sensor cavity or cavities to retain the patient sensor or sensors.

As previously described, in some forms the accessory may be provided as, or as part of, a component of a patient respiratory interface. In particular, an accessory which is referred to as a component of the patient interface may be substitutable for a conventional component of the patient interface. In substituting for the conventional component, the accessory as a component may provide the functionality of the conventional component and in addition provide the sensor-retaining functionality of an accessory.

An accessory as a component may be or be part of a patient interface, such as the patient interface 17 illustrated in FIG. 3. For example, an accessory as a component may be or be part of a headgear of or for a patient interface, for example, as part of the head strap 200 illustrated in FIGS. 7A-H. As a further example, an accessory as a component may be or be part of a side arm of a patient interface, for example, the side arms 31 illustrated in FIG. 4, the side arm 270 illustrated in one or more of FIGS. 7C, 8A and 8B, or the accessory 400 of FIGS. 17A-C or FIGS. 18A-C.

Such a component may replace another part of the patient interface, for example, as has been described in relation to the accessory 400 that can be substituted for a side arm of a patient interface.

In some configurations, a component to which a sensor may be mounted may be an intermediate component between a headgear and a remainder of the patient interface to which it attaches. More particularly, an accessory as a component of the patient interface may be provided as part of a headgear-interface connector, or may include connector features for connecting a headgear and another part of the patient interface together.

Where an accessory is substitutable for a conventional component of a patient interface, the accessory component may interconnect with at least one other part of the patient interface. By interconnecting the accessory component and at least one other component may become interconnected. When interconnected, the components are not able to freely move relative to one another. Contrastingly, in other configurations where the accessory does not interconnect with another component of the patient interface, relative movement of the accessory and/or the part of the patient interface to which it attaches may be possible. For example, the accessory 400 of FIGS. 10A-10C may be configured to in use be slidable along the strap about which it attaches. As a further example, the accessory 400 of FIGS. 16A-E may be configured so that it can slide along the strap which is to be received within the securement feature 610.

An accessory component may be substitutable for, for example, a side arm, a buckle, a tube clip, or any other conventional component of a patient interface. Where the accessory component (hereinafter referred to just as a component) is substituted into the patient interface, the component may connect with other components of the patient interface. For example, where the component is substitutable for a side arm, the component may connect to both the main body of the patient interface and another part of the patient interface such as a headgear strap or headgear connector. The component may interconnect with one or each of the other parts of the patient interface with which it connects.

By way of example, various arrangements of components for a patient interface which are configured to retain a patient sensor will now be described.

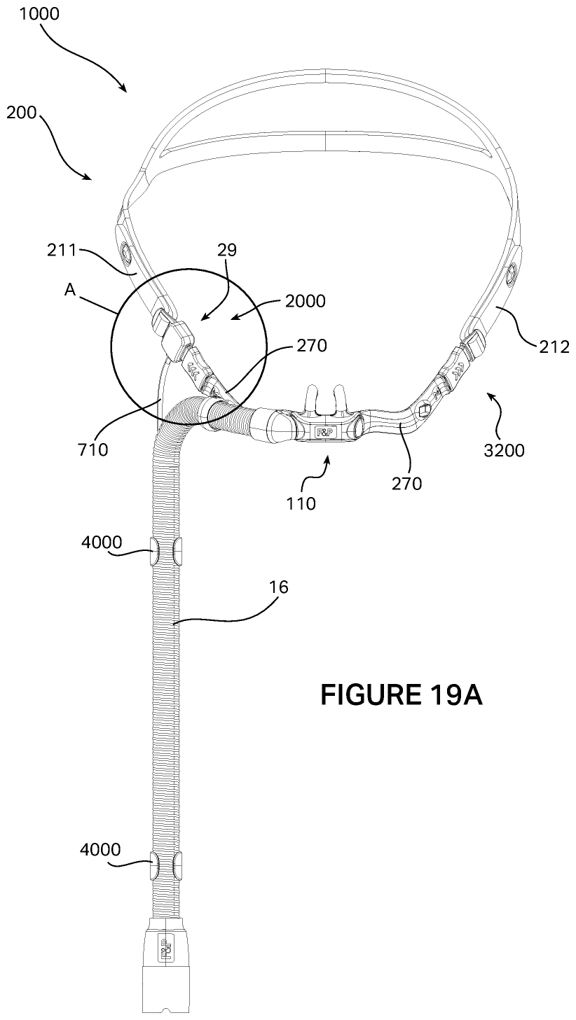

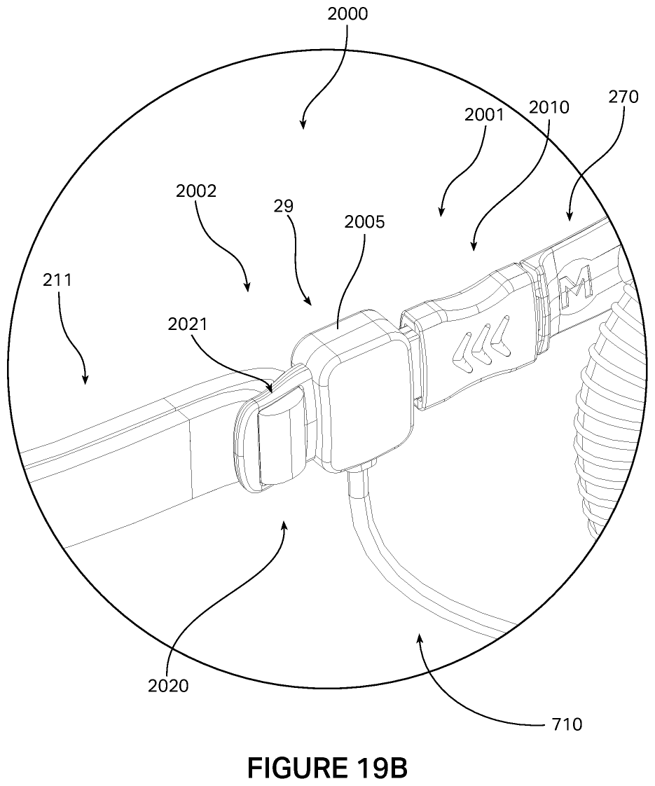

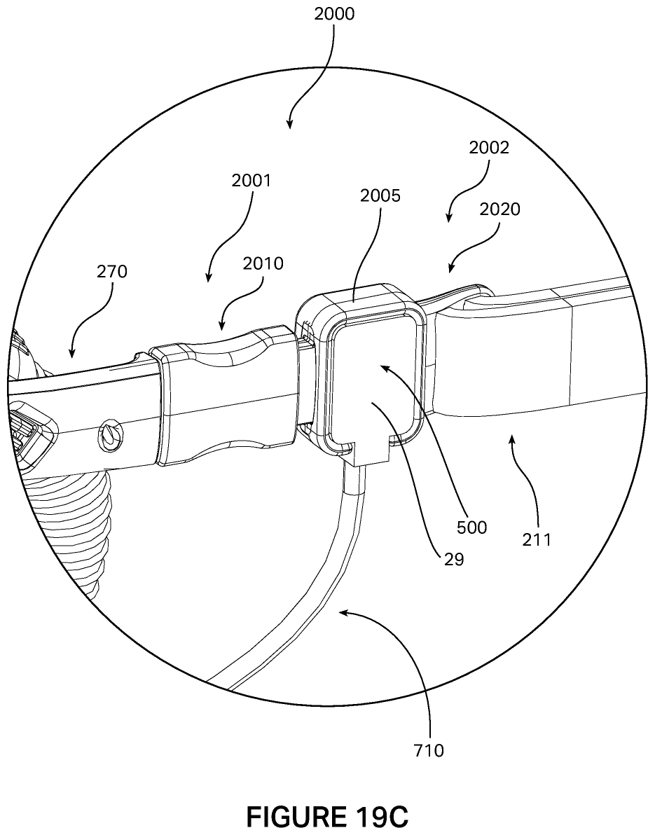

As shown in FIG. 19A, the nasal cannula interface 1000 includes a main body 110. A pair of side arms 270 extend laterally from the main body 110. The nasal cannula interface 1000 also includes a headgear 200, with a first strap 211 and second strap 212.

At one side of the nasal cannula interface 1000 one of the side arms 270 is connected to the second strap 212 of the headgear by a connector 3200. The connector 3200 allows for connection of both the side arm 270 and second strap 212 to it.

At the other side of the nasal cannula interface 1000 of FIG. 19A a component 2000 is provided. The component 2000 is connected to both the other side arm 270 and the first strap 211 of the headgear. The component 2000 also has a component body which can retain a patient sensor 29.

While illustrated in FIG. 19A as only having the component 2000 provided at one side of the connection between the side arm 270 and the headgear 200 of the patient interface, in other configurations a component 2000 may be provided at both sides of the patient interface.

Where a component 2000 is provided at both sides of a patient interface, the patient or a clinician can be provided the option of where a patient sensor is located. For example, the location of a patient sensor may be changed between the components 2000 at either side of their face. This may provide for increased comfort for the patient. It may also allow multiple of the same or different patient sensors to be utilised at the same time.

While illustrated in FIG. 19A as connecting between a side arm 270 and a first strap 211 of the headgear, a component 2000 may connect between any other two desired components of a patient interface. For example, a component 2000 may be provided between two portions of a headgear, or as previously described may be provided in the form of a side arm 270 to connect between a main body of the patient interface and part of a headgear of the patient interface.

A component 2000 may include one or more of the same connector features as a connector that is not configured to retain a patient sensor 29. For example, the component 2000 of the configuration of FIG. 19A may include one or both of the connector features of the connector 3200.

FIG. 19B shows a perspective view of the portion of the patient interface 1000 marked A in FIG. 19A.

In FIG. 19B the component 2000 is shown connected at one end to the side arm 270 and at the other end to the first strap 211 of the headgear. The component 2000 has a first connector 2010 which connects to the side arm 270, and a second connector 2020 which connects to the first strap 211 of the headgear.

As shown in FIG. 19B, the first connector 2010 is located at a first end 2001 of the component 2000 and the second connector 2020 is located at a second end 2002 of the component.

FIG. 19C shows the detail marked A in FIG. 19A, but from the opposite side of the component 2000 as is shown in FIG. 19B.

The component 2000 includes a component body 2005. As is seen in FIG. 19C, the component body has a sensor cavity 500, within which a patient sensor 29 is retained.

The component body 2005 may be located between the first end 2001 and second end 2002 of the component. For example, as shown in FIGS. 19B and 19C, the component body 2005 may be provided between the first connector 2010 and second connector 2020 of the component, so that the first connector 2010 and second connector 2020 are located at respective lateral sides of the component body 2005.

One or both of the first connector 2010 and second connector 2020 may be configured to releasably connect the component with the respective other parts of the patient interface.

For example, as seen particularly in FIG. 19B, the second connector 2020 includes a buckle 2021 through which an end of the first strap 211 of the headgear can be passed. The length of the strap may be adjusted by sliding through the buckle 2021. The first strap 211 may be removed from the buckle 2021 to disconnect the second end 2002 and the first strap 211 from each other.

Also as seen in FIGS. 19B and 19C, the first connector 2010 is configured to be releasably connected to the side arm 270. The first connector 2010 can receive and retain part of the side arm 270. Parts of the first connector 2010 can be moved relative to each other to release the engagement with the side arm 270 and allow the side arm 270 and first connector 2010 to become disconnected with each other.

As illustrated in FIGS. 19A-C, the opening of the sensor cavity 500 is provided at an internal surface of the nasal cannula interface 1000 which in use is adjacent to the patient's face. By this configuration, part of the patient sensor 29 may be directly exposed to the patient's body.

As the opening of the sensor cavity 500 is provided towards the patient's face in use, the patient's face may act to further retain the patient sensor 29 within the sensor cavity 500.

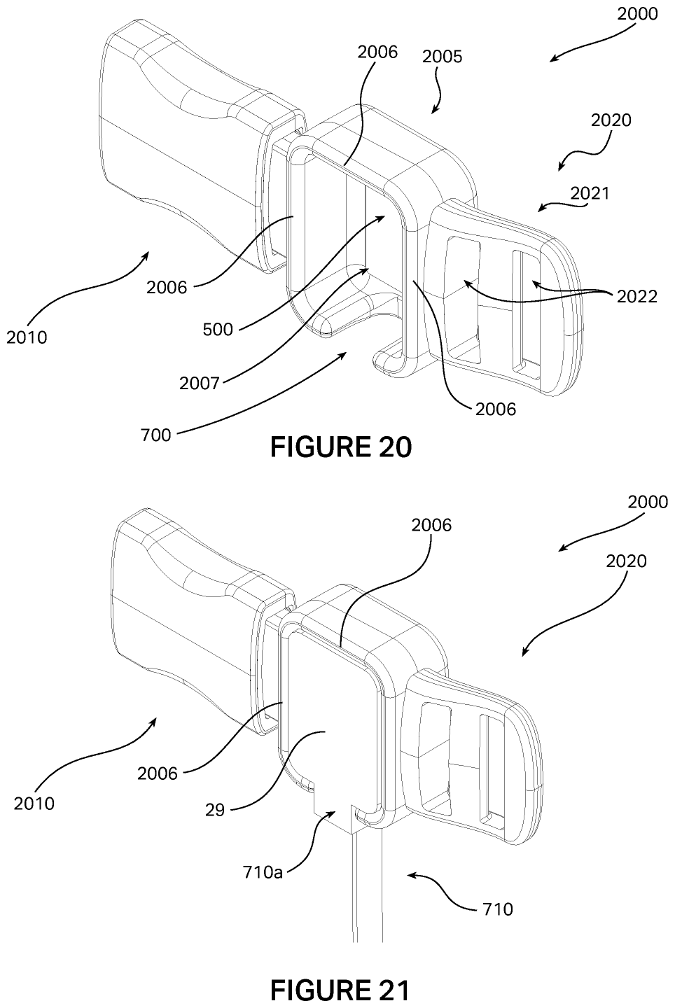

FIG. 20 is a view of a component 2000 not retaining a patient sensor 29, and FIG. 21 is a view of the component 2000 with a patient sensor 29 retained by it.

As shown in FIG. 20, the component body 2005 is located between the first connector 2010 and second connector 2020. The component body 2005 has a sensor cavity 500. In the configuration of FIG. 20, the sensor cavity 500 is formed in a first face 2007 of the component body 2005.

The sensor cavity 500 is defined by one or more walls 2006 of the component body 2005.

The component body includes a wire cavity 700. The wire cavity 700 may extend between the sensor cavity 500 and an outside of the component body 2005 to provide a pathway for one or more wires to the sensor cavity 500.

As previously described, the wire that the wire cavity 700 is configured to provide a pathway for may include a cable, a cord, a lead, cable harness or any other insulated assembly of electrically conductive material.

As seen in FIG. 20, the wire cavity 700 is provided at the first face 2007 of the component body 2005. The wire cavity 700 has an opening at the first face 2007 of the component body 2005. The wire cavity 700 opens between the sensor cavity 500 and an external face of the component body 2005 that is adjacent to the first face 2007.

A wire cavity 700 may take the form of a slot that is recessed from a face of the component body and provides a passageway between the sensor cavity 500 and an outside of the component body.

The wire cavity 700 may be of various cross-sectional configurations. For example, a wire cavity 700 may have a substantially square, rectangular, or circular cross-section. A wire cavity 700 may be of more than one cross-sectional configuration along its length. A wire cavity 700 may have any one of the foregoing cross-sectional configurations at any one or more locations along its length.

As seen in FIG. 20, the buckle 2021 of the second connector 2020 includes two apertures 2022 through which the end of a headgear strap can be passed in to connect the strap to the second connector 2020.

A patient sensor 29 is shown in FIG. 21 retained within the sensor cavity 500 of the component body 2005. A shape of the sensor cavity 500 may be defined to complement a shape of the patient sensor 29. For example, as seen in FIGS. 20 and 21, the perimeter of the sensor cavity 500 around the walls 2006 of the component body corresponds to outer shape of the patient sensor 29.

As seen in FIG. 21, the sensor cavity 500 has a substantially rectangular shape, with rounded edges. A sensor cavity 500 may have various other shapes, such as, for example, a square or a circular shape. The configuration of the sensor cavity, or at least of at least one dimension of the sensor cavity, may correspond to a dimension of an intended patient sensor for use with the component 2000.

A sensor cavity 500 may provide an interference fit with a patient sensor, in order to aid in retaining the patient sensor 29 at or within the sensor cavity 500. Such an interference fit may be in the form of a friction fit. Additionally, or alternatively, an interference fit may include a snap-fit arrangement between the sensor and sensor cavity.

An adhesive may be provided between the patient sensor and part of the sensor cavity to retain the patient sensor within the sensor cavity.

Where the opening of the sensor cavity is to be in use adjacent to the patient's face, the patient's face may also aid in retaining the sensor within the sensor cavity.

A wire cavity 700 may also correspond in shape to the part of the wire 710 which it is to receive. As seen in FIG. 21, the patient sensor 29 includes an extension portion 710a about the proximal portion of the wire 710. The dimensions of the wire cavity 700 are configured to correspond to the dimensions of the extension portion 710a.

The sensor cavity 500 of a component 2000 may be such that when a patient sensor is provided in the sensor cavity, a part of the patient sensor may be exposed to an outside of the component. For example, a patient sensor may include a transducer and/or a light source. The component body 2005 and sensor cavity 500 in it may be arranged so that, when the component 2000 is used as part of a patient interface by a patient, one or both of the transducer and light source faces towards the patient. More particularly, the component body 2005 and sensor cavity may be arranged so that a transducer and/or light source are exposed to an outside of the component 2000.

In some configurations the first face 2007 of the component body 2005 into which the sensor cavity 500 is formed may be a face of the component body which is oriented towards the patient when the component 2000 is used as part of a patient interface 1000.

In such configurations, a body of the patient sensor 29 will be provided adjacent to the patient in use. Accordingly, where the patient sensor 29 includes a transducer and/or light source, these may be provided so that they face towards the patient.

Where the opening of the sensor cavity is oriented towards the patient in use, a patient sensor may be retained or additionally retained in the sensor cavity 500 by the presence of the patient's face.

In other configurations the first face 2007 of the component body 2005 into which the sensor cavity 500 is formed may be a face of the component body which not oriented towards the patient when the component is in use as part of a patient interface 1000. In some configurations, the first face 2007 and opening of the sensor cavity 500 may be oriented away from the patient in use.

In such configurations, the component body 2005 may include one or more holes through the component body 2005 at a wall 2006 of the body to allow a transducer and/or light source of the patient sensor 29 to face towards the patient in use.

The sensor cavity 500 may be arranged to provide part of a retained patient sensor 29 to be in contact with the patient when the component 2000 is used as part of a patient interface.

An opening of a wire cavity 700 may be oriented to face the patient in use. More particularly, an opening of the wire cavity 700 in a direction transverse to the length of wire cavity 700 between the sensor cavity 500 and the outside of the component body 2005 may face the patient in use.

The patient-facing face of the component body 2005 of the component 2000 illustrated in FIGS. 19A and 19B is substantially rectangular. In other configurations, a patient-facing face of a component body 2005 may have a substantially circular shape.

A component 2000 or at least the component body 2005 and any parts integrally formed therewith may be formed from a biocompatible material. For example, they may be formed from a biocompatible plastics material, such as for example a thermoplastic elastomer (TPE), or silicone, or a liquid silicone rubber (LSR).

As previously described in relation to the accessory 400, and particularly with reference to FIG. 16E, a face of the component 2000 or particularly the component body 2005 which is configured to face the patient in use may comprise at least one surface material, such as the surface material 227 previously described. The surface material may be provided across part or all of the face of the component 2000 which faces the patient.

The surface material may be the same material as what the component 2000 or particularly component body 2005 is made from. In other configurations, the surface material may be a different material.

A surface material of the component 2000 may function to provide a desired coefficient of friction between the component 2000 and the patient's face.

A surface material of the component 2000 may act to provide retaining of the sensor within the sensor cavity. For example, a surface material may be provided across part or all of the opening of the sensor cavity once the sensor has been provided therein.

A patient sensor 29 may be provided within the sensor cavity 500 as part of a component 2000. In other configurations, an appropriate patient sensor 29 may be fitted to the component 2000 by a user.

A component 2000 may be provided as a separate element for use with a patient interface. Alternatively, a component 2000 may be provided as part of a patient interface.

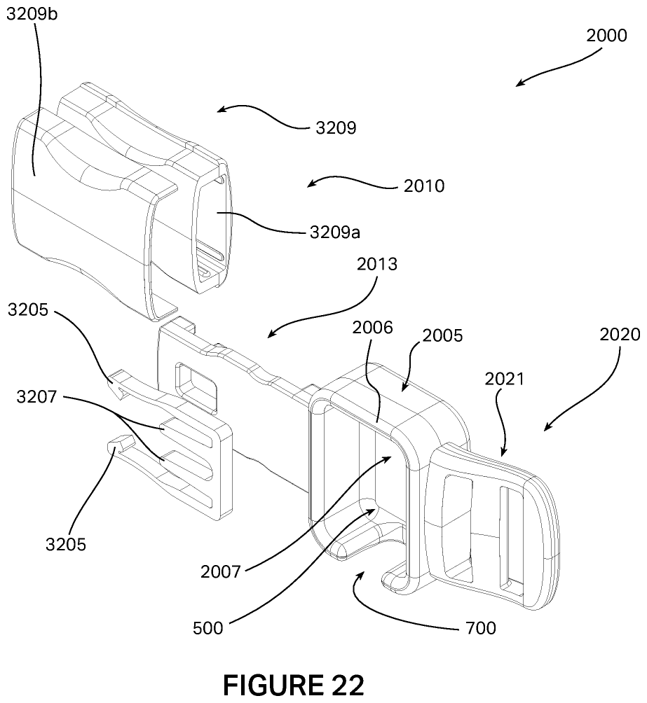

The exploded view of FIG. 22 shows various components of a configuration of a component 2000 that includes a first connector 2010 and a second connector 2020. As seen in FIG. 22, the second connector has a buckle 2021 to which a strap of a headgear may be attached.

The first connector 2010 includes a first connector body 2013. In the configuration of FIG. 22 the first connector body 2013 is formed integrally with the component body 2005. The component body 2005 is also formed integrally with the second connector 2020.

In the exploded view of FIG. 22, the first connector 2010 also includes a second body having a pair of detents 3205 and biasing means 3207. The first connector 2010 also includes a slide 3209, which has a first slide part 3209a and a second slide part 3209b. The parts of the first connector 2010 may cooperate to allow another part of the patient interface, such as an end of a side arm 270, to be received and retained by the first connector then to be selectively released.

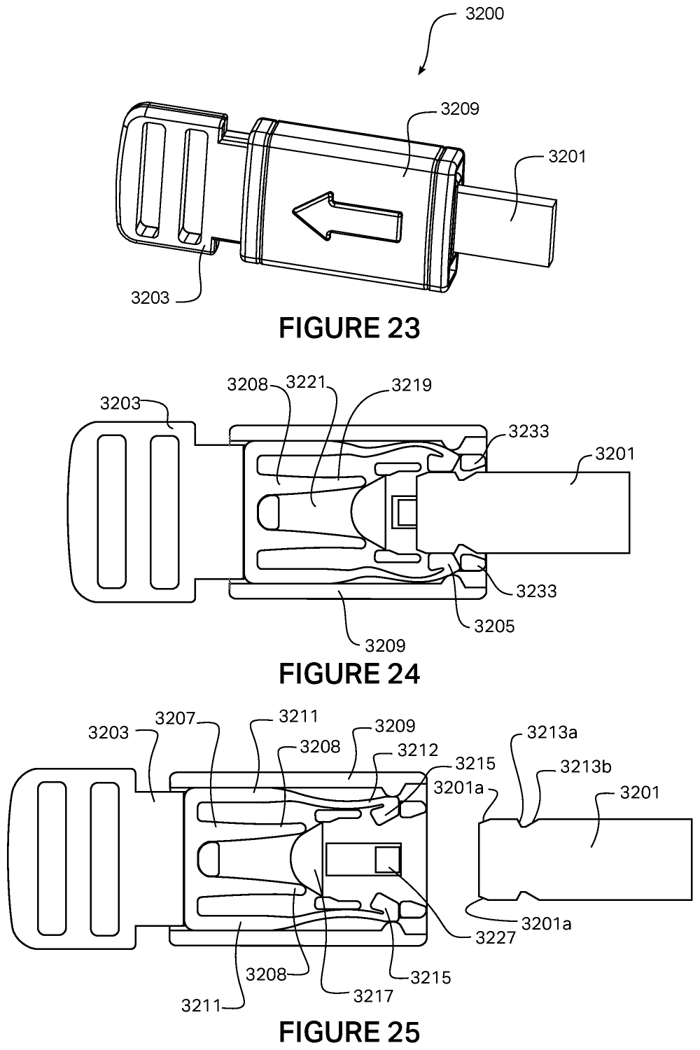

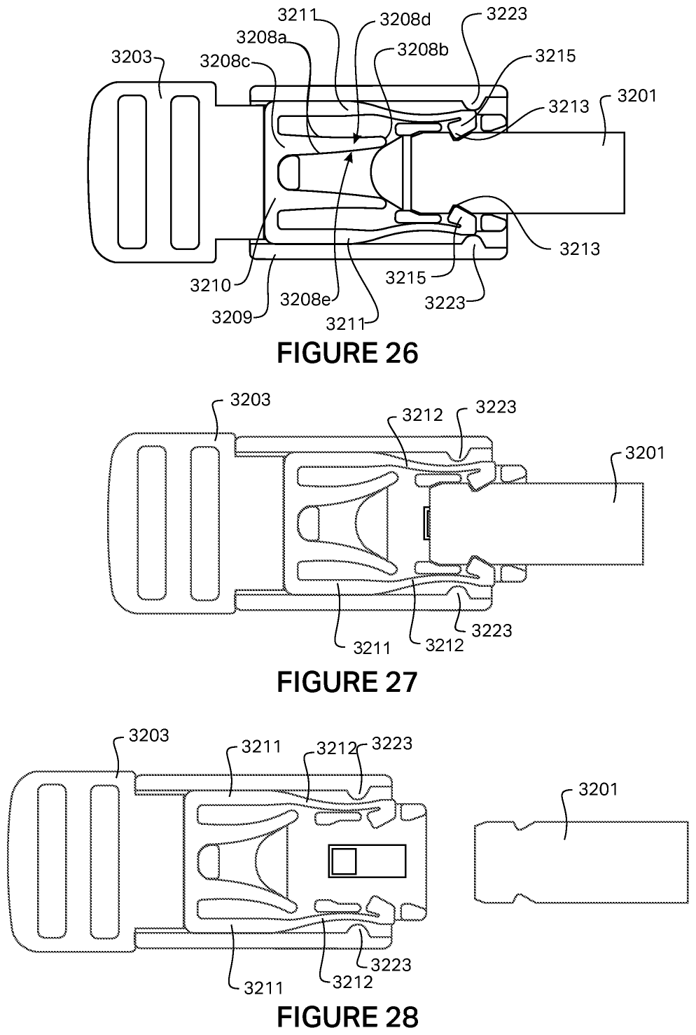

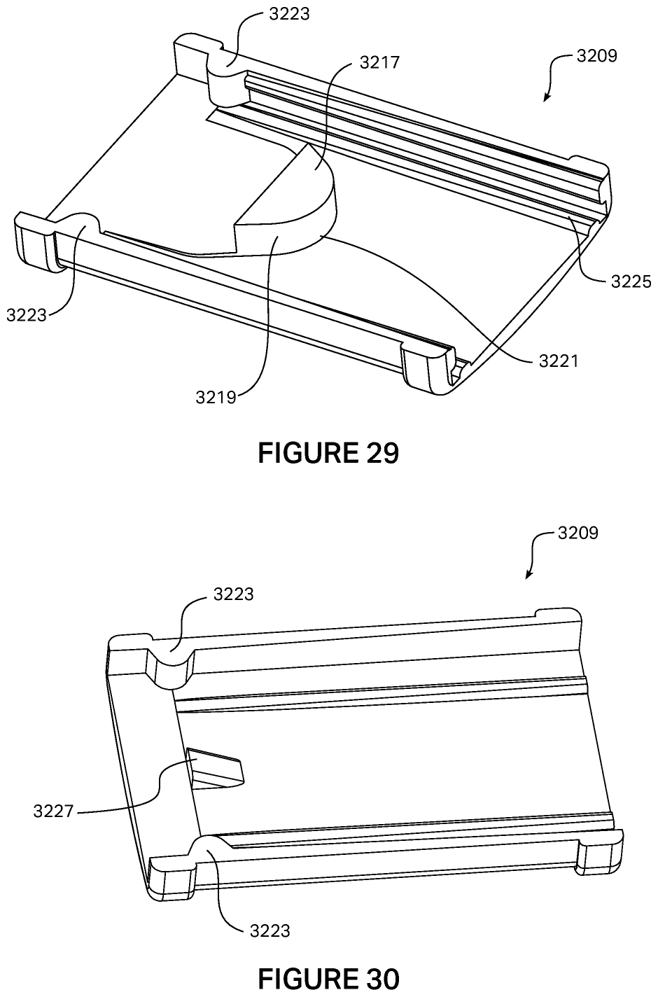

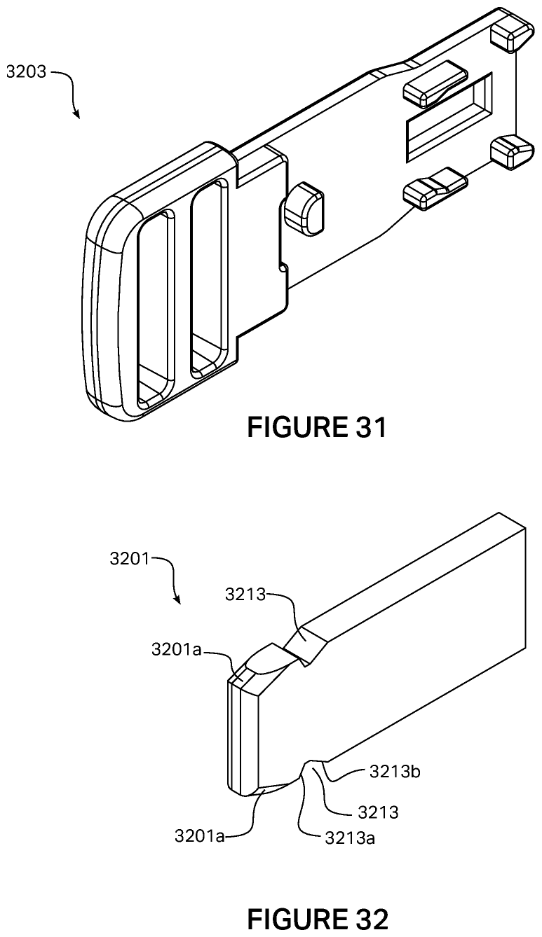

A component 2000 may have the connector features of a connector 3200, as is illustrated in FIGS. 23-32. Further details of the configuration of a first connector 2010 of a component 2000 are provided below by way of description of a connector 3200.

Described in relation to FIGS. 23-32 is a first connector part 3201 which can be secured to the connector 3200 by the detent 3205 and/or protrusion 3215. While described in relation to FIGS. 23-32 as a first connector part 3201 or clip 3301, reference elsewhere herein will be made to a first connector. A first connector may be generally understood as relating to a part the patient interface where a first connection with another part of the patient interface, for example, a body of the patient interface, may be made. For example, in FIGS. 19A and 19B, the component 2000 has a first connector 2010 which connects to a body of the patient interface 1000. More particularly, the first connector 2010 connects to a side arm 270 of the patient interface 1000.

Described in relation to FIGS. 23-32 is a second connector part 3203, as part of the connector 3200. Such a part is elsewhere herein described as a second connector. A second connector may generally be understood as relating to a portion of a component of the patient interface where a second connection with another part of the patient interface, for example, with a headgear or headgear part, may be made. For example, in FIGS. 19A and 19B the component 2000 has a second connector 2020 which connects to a headgear 200 of the patient interface.

With reference to FIGS. 23 to 32, a first preferred embodiment of a connector 3200 will now be described. The connector 3200 has a first connector part in the form of a clip 3201, a second connector part in the form of a carrier 3203, a detent 3205 for securing the clip and the carrier together, a biasing means 3207, and a slide 3209. The slide 3209 is moveable relative to the clip 3201 and/or the carrier 3203 between a secured position and a free position. In the secured position, the detent 3205 is substantially inhibited from moving and releasing the clip 3201 from the carrier 3203. In the free position, the detent 3205 is able to move to release the clip 3201 from the carrier 3203. The biasing means 3207 urges the slide towards the secured position.

In the preferred embodiment shown, the biasing means 3207 and detent 3205 are integrally formed together. The biasing means 3207 comprises a pair of resilient legs 3208 and the detent comprises a pair of resilient arms 3211. The arms and legs are substantially resistant to deformation or are resiliently flexible. The arms 3211 are flexible to allow the clip 3201 to be inserted when the slider 3209 is in the secured position, as described below. The arms and legs extend from a body portion 3210.

As described in more detail below, the resilient legs 3208 urge the slide towards the secured position. The legs extend from the body portion 3210 in the same direction and are the same length as each other. The sides 3208a of each leg are slightly tapered so that a free end 3208b of each leg is narrower than an end 3208c that joins the body portion 3210. The free end 3208b of each leg is rounded. The side surfaces, top surface 3208d and bottom surface 3208e of each leg are substantially planar surfaces.

The resilient arms 3211 are spaced apart and extend from the body portion 3210 in the same direction as the legs 3208. The arms are the same length as each other, and longer than the legs. The arms 3211 are biased towards each other. As described in more detail below, when the clip 3201 is inserted into the carrier 3203, the pair of resilient arms 3211 are biased towards engagement with the notches of the clip.

Each arm has a linear portion closest to the body portion 3210 which extends into an arcuate portion 3212. The arcuate portion 3212 allows the arms 3211 to bend when the clip 3201 is inserted into the carrier 3203 with the slide 3209 in the secured position. The arcuate portion 3212 is concave when viewed from the position of the slide. The arcuate portion 3212 has a narrower width than the linear portion. The side surfaces, top and bottom surfaces of each arm are substantially planar surfaces. The top and bottom surfaces act as bearing surfaces between the slide 3209 and the subassembly of the carrier with the biasing means 3207/detent 3205.

Each resilient arm 3211 comprises a protrusion 3215 for engagement with a complementary notch of the clip 3201. The protrusion 3215 is at the free end of each arm. Each protrusion 3215 has a generally triangular shape, as shown in FIG. 24 and the notch has a complementary triangular shape for positive engagement between the protrusion and the notch. In an alternative embodiment, the notch may be a shoulder.

The slide 3209 has a lug 3217 for engagement with the biasing means and a stop 3227 as shown in FIG. 25 for locating the slide and carrier 3203 in the secured configuration. Another stop may be provided near stop 3227 to control the extent of movement of the slider in the free position. There may be an additional stop or stop 3227 may be extended. In the preferred embodiment shown, the slide 3209 is a sleeve. The sleeve has a first interior surface and a second spaced apart interior surface opposite the first surface. The stop 3227 is formed on the first surface and the lug 3217 is formed on the second surface. In an alternative embodiment, the stop and lug may be formed on the same surface. FIGS. 24 to 28 are cross sectional views through a central plane of the connector. Accordingly, those figures show the lug, the stop, and the biasing means.

As shown in FIGS. 24 to 28, the lug 3217 is centrally positioned within the slide 3209. The lug 3217 has two outwardly tapered surfaces 3219 with a rounded nose 3221.

The stop has a wedge-shaped profile when viewed from the side, as shown in FIG. 30. The stop cooperates with a slot in the carrier. When the slide is in the secured position, a surface of the stop engages a surface of the slot, preventing the slide from moving past the secured position. The wedge shape of the stop aids assembly of the slide to the carrier.

The slide 3209 has two protrusions 3223 for engagement with the detent for substantially inhibiting movement and release of the clip 3201 from the carrier 3203. The protrusions are generally semi-circular shaped, as shown in FIGS. 24 to 28. The slide 3209 has a pair of longitudinally extending rails 3225. The slide may have scalloped portions for a user to grasp the slider. The slider may be fitted with a soft sleeve component to provide the user with a better grip to operate the connector, which may also be more comfortable for a patient to have against their skin. The sleeve may be overmolded or co-moulded with the sleeve or may be a separately formed component that is assembled with the slider.

The clip 3201 is a substantially planar and rigid component. The clip 3201 comprises a pair of notches 3213. The notches 3213 are positioned towards a nose section 3201a of the clip 3201 and are a generally triangular shape. A first side 3213a of the notch 3213 closest to the nose of the clip 3201 is a steeper angle than the second side 3213b of the notch 3213. The first side is relatively steep to assist in preventing the clip 3201 from being removed when the slide is in the secured position.

The carrier has been described as a separate component to the combined detent/biasing means component. Alternatively, the carrier may be integrally formed with the detent/biasing means component.

The biasing means has been described as comprising a pair of resilient legs. Alternatively, the biasing means may comprise a single leg or more than two legs. In further alternatives, the leg biasing means may comprise any other type of spring element to act as a return mechanism for the slider.

The detent has been described as comprising a pair of resilient arms. Alternatively, the detent may comprise a single arm or more than two arms. The clip has been described as having a pair of notches. Alternatively, the clip may have a single notch or more than one notch. The number and position of the notches will correspond to the number and position of the complementary protrusions on the resilient arms.

The biasing means has been described as having two legs that move away from each other and are biased towards each other to urge the slider to the secured position. Alternatively, the legs may be deformed in another direction to provide a similar return action. For example, the legs may twist or bend along their length.

The features and characteristics of the legs may be modified to suit the application; that is, they can be modified to tune the force of on the slide as it moves between the secured and free position. The features and characteristics that may be chosen or designed to be modified include the angle of the legs, the thickness of the legs, and the angle of the lug.

In a further alternative embodiment, the combined detent/biasing means component may be formed in two parts. Each part would have an arm and a leg and would be held in place relative to the carrier by location features. In a further alternative embodiment, the detent/biasing means may be a single leg and arm combination that act on one side of the clip only. In this embodiment, the connector will have location features for securing the detent/biasing means in place together with guides and/or abutment features to ensure the components of the connector are correctly located relative to each other in view of the forces acting on the components by the arm/leg.

The biasing means and detent have been described as being integrally formed together. Alternatively, they may be separately formed components that may or may not be connected together.

The embodiments of the connector have been described as having a biasing means for urging the slide towards the secured position. In an alternative embodiment, the connector may not have a biasing means, but the slider could be held in the free and/or secured positions by other suitable mechanisms. For example, the connector may have one or more catches that hold the slider in the free and/or secured positions. Such catches may automatically engage the slider and/or carrier or may be features that are controlled by a user.

The disclosure also provides for a conduit and wire clip 4000. The clip 4000 may be used to attach a conduit of a patient interface together with a wire, for example the wire of a patient sensor 29.

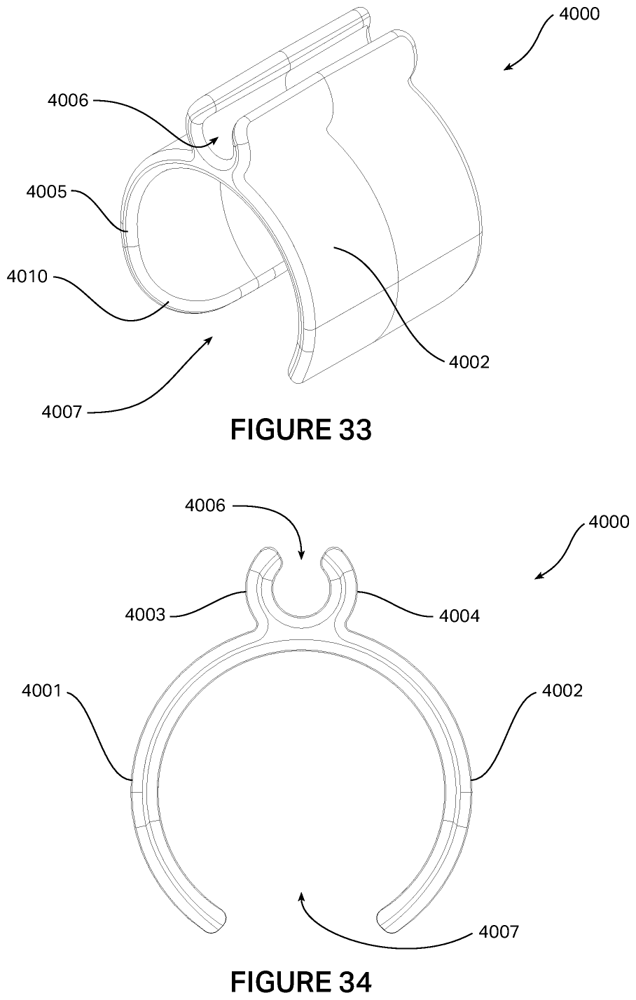

FIG. 33 illustrates a perspective view of a clip 4000, and FIG. 34 illustrates an end-on view of the clip 4000. The clip 4000 has a wire receptacle 4006 to receive a wire and a conduit receptacle 4007 to receive a conduit.

The conduit receptacle 4005 is defined by the two walls 4001 and 4002. The wire receptacle 4006 is defined by the two walls 4003 and 4004. Each of the receptacles have an opening across their length which is narrower than a corresponding maximum width of the receptacle. The pairs of walls 4001 and 4002, and 4003 and 4004, each curve towards each other at their distal extents.

The cross-sectional size and shape of each receptacle may be made to correspond to the cross-sectional size and shape of an intended sensor wire and conduit, respectively.

The clip 4000 of FIGS. 33 and 34 may be for connection to a wire and conduit which each have a substantially circular cross-section.

The width of each of the receptacles 4006 and 4007 may be made to be the same or preferably smaller than a corresponding width of the wire or conduit with which the clip is to be coupled. By this configuration, an interference fit may be provided between the wire or conduit and the clip when the respective element is located within a respective receptacle.

While illustrated in FIGS. 33 and 34 as having a length that is approximately equal to its overall lateral dimensions, a clip 4000 may have either relatively greater or lesser length than one or both of its lateral dimensions.

As seen in FIG. 33, the corner 4010 of the first wall 4001 is rounded between its edge at the opening of the conduit receptacle 4007 and its edge at the proximal end of the clip 4000. The rounded corner provides a locally increased width of the opening of the conduit receptacle 4007. This locally increased width may aid in a user attaching the clip onto the conduit.

A corresponding corner of the other wall 4002 of the conduit receptacle 4007 may be rounded in the same way, to further increase the width of the opening of the conduit receptacle 4007 at its end.

Only one end of the conduit receptacle 4007 of a clip 4000 may have rounded corners, or both ends may.

Rounded corners may similarly be provided on one or more of the corners or one or both ends of the wire receptacle 4006 of a clip 4000.

One or more clips such as the clips 4000 may be used along with a patient interface, and particularly with a patient interface that can retain a patient sensor as described herein. Where a patient sensor includes a wire, the trailing wire may present an inconvenience to the patient or a clinician. By the use of one or more clips, one or more sensor wires may be secured to a conduit of the patient interface. This may reduce clutter and increase the simplicity of the overall patient interface and patient or clinician interactions with it.

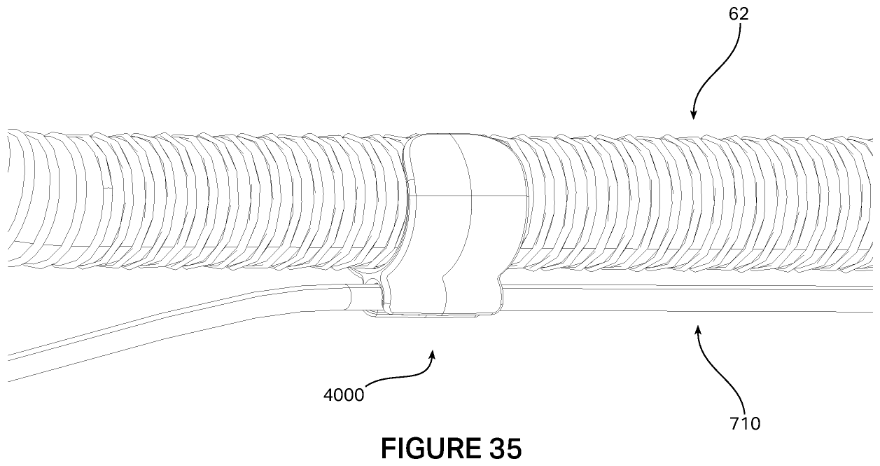

FIG. 35 shows part of a conduit 62 for a patient interface, along with a wire 710, for example for a patient sensor. A clip 4000 is attached to both the conduit 62 and wire 710, so that a portion of the conduit 62 is received with a conduit receptacle of the clip and a portion of the wire 710 is received within a wire receptacle of the clip.

As seen in FIG. 35, the clip 4000 orients the longitudinal axes of the conduit 6 and wire 710 parallel with each other, at the location where the clip 4000 is connected.

FIG. 19A also illustrates two clips 4000 as part of the patient respiratory interface 1000 with a conduit 16. In FIG. 19A the opening of each of the conduit receptacles are shown. The wire 710 that extends from the patient sensor extends along the length of the conduit 16 and is obscured by it.

In some arrangements, a component of or for a patient interface that is configured to retain a patient sensor may be a connector that includes a conduit retaining portion or tube clip. The component may have a sensor mount that is configured to retain the patient sensor. The sensor mount may be configured to couple a sensor mount receiving portion of another component of the patient interface.

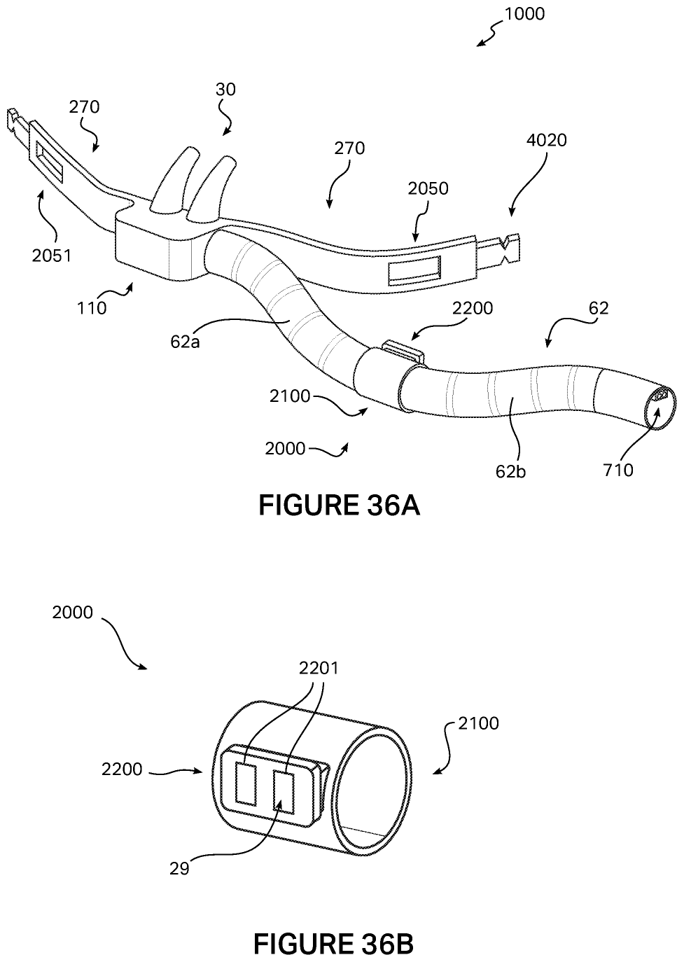

FIG. 36A illustrates a patient respiratory interface in the form of a nasal cannula interface 1000. While illustrated as a nasal cannula interface, it will be appreciated that the interface may be of other types as disclosed herein. The nasal cannula interface 1000 has a nasal cannula 30 and associated gases inlet conduit 62 for supplying a flow of breathable gases. The nasal cannula interface 1000 has respective side arms 270 which extend on either lateral side of the nasal cannula 30.

In some arrangements, the gases inlet conduit 62 may be provided as a first portion 62a and a second portion 62b. The first portion 62a and second portion 62b may be connected together at or by the component 2000, and more particularly at or by the conduit retaining portion 2100.

The nasal cannula interface 1000 includes a component 2000 which is configured to retain a patient sensor 29. The component 2000 is provided in the form of a component 2000. The component 2000 has a conduit retaining portion 2100 for retaining a portion of the gases inlet conduit 62. The component 2000 has a sensor mount 2200 which is configured to retain a patient sensor 29.

The conduit retaining portion 2100 may have a shape which surrounds an entirety of the gases inlet conduit 62, as illustrated in FIG. 36A in the form of a collar which surrounds the gases inlet conduit 62. In other arrangements, the conduit retaining portion 2100 may be in other forms which do not entirely surround the gases inlet conduit 62. For example, the conduit retaining portion 2100 may be in the form of a tube clip or part thereof as described elsewhere herein, for example in relation to FIGS. 33-35.

In some arrangements, the conduit retaining portion 2100 may be removably attachable to the gases inlet conduit 62. For example, where the conduit retaining portion 2100 is in the form of a conduit receptacle 4007 of the tube clip 4000 of FIGS. 33-35, the conduit retaining portion 2100 may be removably attachable to the gases inlet conduit 62.

In other arrangements, such as for example where the conduit retaining portion 2100 entirely surrounds the conduit, the conduit retaining portion 2100 may only be removable by sliding over an end of the gases inlet conduit 62 or may not be non-destructively removable from the conduit.

In some arrangements, the conduit retaining portion 2100 when retaining the gases inlet conduit 62 may be fixedly associated with the conduit. In such arrangements, the conduit retaining portion may not be able to move relative to the gases inlet conduit, for example by either or both of a translation along the gases inlet conduit or a rotation about it. In such arrangements, the component 2000 may be provided as part of the gases inlet conduit 62.

In other arrangements, the conduit retaining portion 2100 may be at least partially movably associated with the gases inlet conduit 62, for example by being one or both of translatable along the gases inlet conduit or rotatable about it.

At least one side arm 270 of the patient interface has a sensor recess 2050. The sensor recess 2050 is sized and configured to receive the sensor mount 2200 of the component 2000. The sensor recess is configured so that, when the patient interface is worn by a patient, a portion of the patient's face is optically exposed to a patient sensor 29 retained in the sensor mount 2200 which is itself inserted into the sensor recess 2050.

The sensor recess 2050 may be configured and located on the side arm 270 so that a portion of the patient's cheek is at least optically exposed to a sensor mount 2200 inserted therein. More particularly, the sensor recess 2050 may be configured and located on the side arm 270 so that a portion of the patient's face between an eye and their lip is at least optically exposed through the sensor recess.

In some arrangements, the sensor recess 2050 may be a windowed recess in the side arm. The sensor recess may extend into the side arm from a non-patient-facing side of the side arm 270, and an at least partially transparent window may be provided at the base of the sensor recess 2050. The sensor mount 2200 may be insertable into the sensor recess 2050 up to the window of the recess.

In other arrangements, for example as illustrated in FIG. 36A, the sensor recess 2050 may be an aperture that extends through the side arm 270 between the non-patient-facing and patient-facing sides of the side arm 270. In these arrangements, a portion of the patient's face beneath the sensor recess 2050 is physically exposed when the patient interface 1000 is worn. The sensor recess 2050 is arranged such that when the patient interface is worn by a patient, the sensor recess exposes a portion of the patient's face which would otherwise have been covered by the interface.

Where the sensor recess 2050 is in the form of an aperture through the side arm 270, a sensor mount 2200 inserted into the sensor recess 2050 may, when the patient interface is worn by a patient, be arranged to be in contact with the patient's face.

The sensor mount 2200 and sensor recess 2050 may engage with an interference fit when the sensor mount 2200 is inserted into the sensor recess 2050.

In some arrangements, the sensor mount 2200 and sensor recess 2050 may be configured to become engaged with each other so as to resist the withdrawal of the sensor mount 2200 from the sensor recess 2050 once the sensor mount 2200 is inserted a particular degree or fully into the sensor recess 2050.

The sensor mount 2200 and sensor recess 2050 may have mutual engagement features which engage together upon insertion of the sensor mount 2200 to a predetermined depth within the sensor recess 2050. The predetermined depth may be such that, when the patient interface is worn by a patient, the sensor mount is in contact with the patient's face. Such mutual engagement features may, for example, be snap-fit features, interference fit features, or magnetic engagement features.

In some arrangements the sensor mount 2200 may be configured to become irreversibly connected with the sensor recess 2050 when inserted therein. In other arrangements the sensor mount 2200 may be resisted against removal when inserted into the sensor recess 2050 but may be repeatably insertable and removable therefrom.

The sensor recess 2050 of a side arm may be of any shape, but particularly one which corresponds to the shape of the sensor mount 2200 which it is to receive. For example, the sensor mount 2200 may have a square, rectangular, ovoid, or circular cross-section, and the sensor recess 2050 may have a corresponding square, rectangular, ovoid, or circular cross-section.

In some arrangements it may be desirable to limit or prevent rotation of the sensor mount 2200 and sensor recess 2050 relative each other about an axis of insertion of the sensor mount 2200 into the sensor recess 2050. In these cases, the cross-sections of the sensor mount 2200 and sensor recess 2050 may closely correspond with each other and may be of a not solely circular shape. A not solely circular shape may include a circular cross-section with one or more detents or notches and corresponding projections, to act together in use to resist relative rotation between the parts.

The side arm 270 with a sensor recess 2050 may be configured to be substitutable for a conventional side arm of another patient interface, to provide for the integration of sensing of one or more patient parameters with the patient interface. Where sensing of a patient parameter is desired, a user may disconnect the existing side arm from the patient interface and headgear or headgear connector and reconnect in its place the side arm 270 with a sensor mount 2200. Where the patient interface includes a component 2000, the sensor mount 2200 may then be inserted into the sensor recess 2050 for use in monitoring the patient parameter. Where a particular patient interface does not include a component 2000, a component 2000 may be provided to the gases inlet conduit 62, or otherwise an gases inlet conduit 62 having a component 2000 may be provided for connection to the patient interface, and the sensor mount 2200 then inserted into the sensor recess 2050.

Because the sensor mount 2200 is insertable into the sensor recess 2050 from the side of the side arm 270 which contacts the patient's face in use, the sensor mount 2200 may be able to be inserted and removed from the sensor recess 2050 while the patient interface 1000 is in use by a patient.

As illustrated in the configuration of FIG. 36A, both of the side arms 270 of the nasal cannula interface 1000 have respective sensor recesses 2050 and 2051. Such a configuration may allow for a sensor mount 2200 to be selectively inserted into either sensor recess 2050 or 2051, such that the patient sensor 29 may be positioned at either side of the patient's face.

In the configuration illustrated in FIG. 36A, the patient gases conduit 62 away from its connection to the main body 110 may be curved around to the other side of the interface, so that the illustrated sensor mount 2200 of the component 2000 may be inserted into the sensor recess 2051.

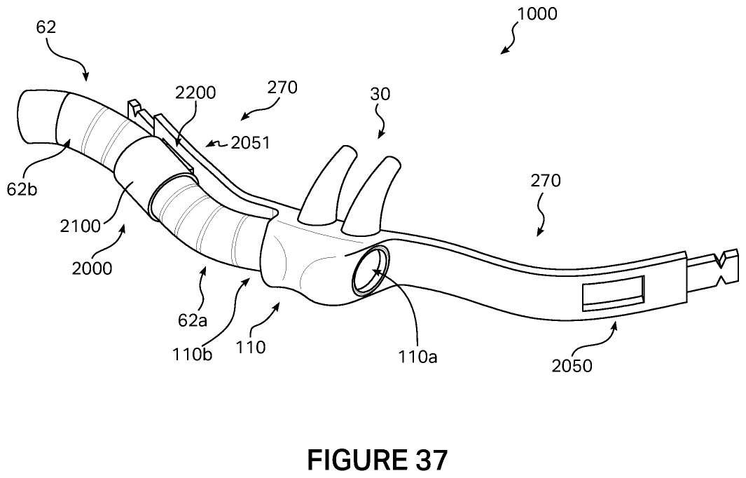

In other arrangements, such as for example illustrated in FIG. 37, the patient interface 1000 may be configured so that the gases inlet conduit 62 is optionally connectable to either lateral side of the main body 110 of the interface. In such an arrangement the gases inlet conduit may be said to be connectable to the main body in a plurality of different directions. In the configuration of FIG. 36A, the conduit 62 is connected to the left side of the main body 110 from the patient's perspective. As shown in FIG. 37 the main body 110 has connection ports 110a and 110b for the gases inlet conduit 62 at both the left and right sides of the main body 110, and the gases inlet conduit 62 is shown connected at the right side of the main body 110 at the connection port 110b. When connected at the connection port 110b, the sensor mount 2200 of the component 2000 may be insertable into the sensor recess 2051 of the right side arm 270. The connection port 110a which does not have the gases inlet conduit 62 connected to it may be sealed by a plug or other closure.

This arrangement allows a user to conveniently select the side of the patient's face at which the patient sensor 29 is provided by switching the side of the main body 110 at which the gases inlet conduit 62 is connected.

By connecting the gases inlet conduit at the side of the main body 110 which corresponds to the sensor recess 2050 or 2051 to which the sensor mount 2200 is to be inserted, an amount of curvature of the gases inlet conduit may be minimised. In particular, the curvature of the gases inlet conduit 62 between a first end at which it connects to the main body 110 and where the conduit is retained by the conduit retaining portion 2100 may be less than about 90 degrees. More particularly, the curvature may be less than about 45 degrees.

Further detail of the component 2000 is shown in FIG. 36B. The component 2000 has the conduit retaining portion 2100 and sensor mount 2200. The sensor mount 2200 is configured to retain a patient sensor 29. The patient sensor 29 may be removably retained by the sensor mount 2200. In other forms, the patient sensor 29 may be irremovably retained by the sensor mount 2200, for example by being integrally formed with it. The sensor mount 2200 may function as a partial housing within which the patient sensor 29 is retained. The sensor mount 2200 may have one or more openings 2201 through which the patient sensor 29 may be exposed to the patient when the sensor mount 2200 is inserted into the sensor recess 2050 of the side arm 270.

The gases inlet conduit 62 for being retained by the component 2000 as illustrated and described in relation to FIGS. 36A-37 may include one or more conductive elements. The conductive elements may extend along part or all of the length of the gases inlet conduit 62. For example, in some arrangements one or more conductive elements may extend from a first end of the tube for connection to the main body 110 of the patient interface 1000 and a second end opposite of the first end, which may be for connection to an inspiratory conduit 16 as illustrated in FIG. 3. In other arrangements, one or more conductive elements may extend partially along the length of the gases inlet conduit from one of its ends. For example, one or more conductive elements may extend from the second end of the gases inlet conduit 62 to a location along the gases inlet conduit at which the conduit retaining portion 2100 is configured to retain the conduit. Where the gases inlet conduit 62 has a first portion 62a extending from the first end and second portion 62b extending from the second end, the one or more connective elements may be associated with only one of the first portion 62a and second portion 62b. One or more of the conductive elements associated with the gases inlet conduit 62 or at least a portion thereof may be for operation of the patient sensor 29. For example, an electrically conductive element may provide power to the patient sensor 29, and/or receive sensor signals from the patient sensor 29.

The one or more conductive elements may be provided in a bead or a lumen of the conduit. In other examples, the conductive elements may associated with the conduit but located externally of the conduit. The one or more conductive elements may for example comprise one or more wires, cables, leads, or other insulated electrically conductive materials.

FIG. 36A illustrates conductive elements 710 which are associated with the gases inlet conduit 62. In the configuration illustrated in FIG. 36A, one or more of the conductive elements 710 may extend only in the second portion 62b of the gases inlet conduit 62 and may interface with the component 2000 and more particularly with a patient sensor 29 retained in the sensor mount 2200 of the component.

Where a gases inlet conduit 62 has a first portion 62a and second portion 62b, the component 2000 having the conduit retaining portion 2100 and sensor mount 2200 may be provided at an interface between the first portion 62a and second portion 62b. The conduit retaining portion 2100 may itself interface between the first portion 62 and second portion 62b of the gases inlet conduit 62. The first portion 62a and second portion 62b may interface with each other when the interface is worn by a patient at a location above the patient's chest, near the patient's face, near the patient's cheek, or more particularly between an eye and the mouth of the patient.

Where a gases inlet conduit 62 has a first portion 62a and a second portion 62b and the component 2000 is located at or provides their interface, the length of the first portion 62a may be such as to position the sensor mount 2200 adjacent to the sensor recess 2050 of a side arm 270 which is located laterally an adjacent connection port of the main body 110 for the conduit 62.

While illustrated in FIG. 36A-37 as being formed in one or more side arms 270, one or more sensor recesses may additionally or alternatively be provided in one or more other components of the patient interface. For example, one or more sensor recesses may additionally or alternatively be provided in a strap of a headgear, or in a headgear to side arm connector component, for example as has been described in relation to FIGS. 23-32.

As described in relation to the component 2000 of FIGS. 36A-37, in different arrangements the conduit retaining portion may be moveable along the gases inlet conduit 62 or may be fixedly associated with a particular location along the gases inlet conduit 62.

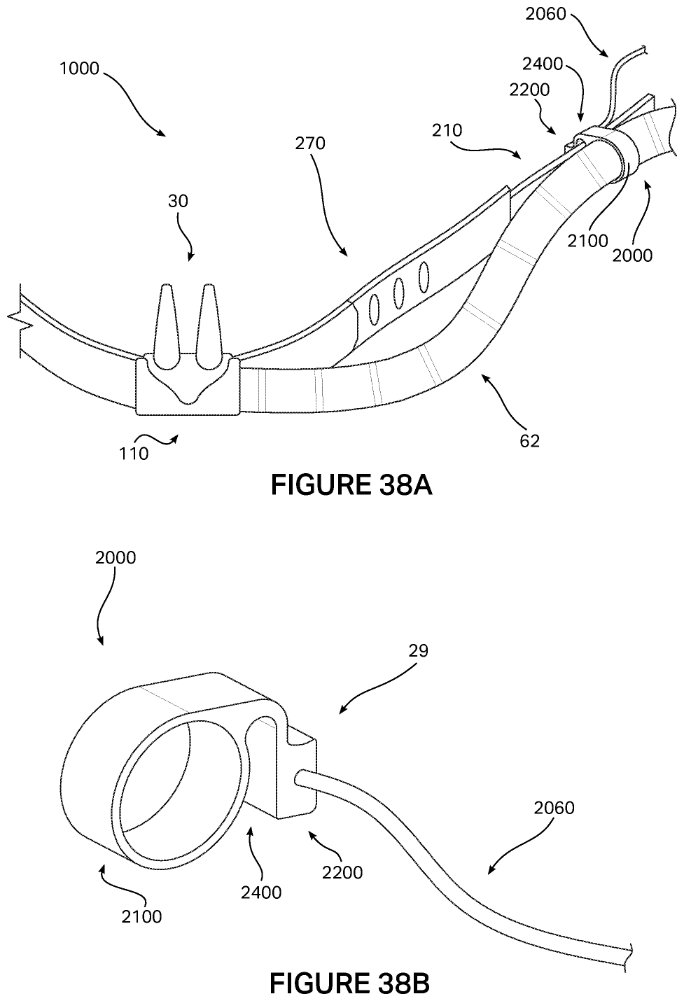

FIG. 38A illustrates a partial view of a nasal cannula interface 1000 with another configuration of a component 2000 for retaining a patient sensor 29. The nasal cannula interface 1000 includes a cannula 30 associated with a main body 110. A side arm 270 is shown associated with one side of the main body 110, and a gases inlet conduit 62 is connected to the main body 110 to supply breathable gases. The component 2000 retains the gases inlet conduit 62 at a conduit retaining portion 2100. The component 2000 has a hook portion 2400 at which the component 2000 can be appended to another part of the patient interface 1000. For example, the hook portion 2400 may be passed over a side arm or headgear strap to associate the component 2000 with the rest of the patient respiratory interface 1000. As illustrated in FIG. 38A, the hook portion 2400 is located over a headgear strap 210. Further detail of the component 2000 including its hook portion 2400 is illustrated in FIG. 38B. On the opposite side of the hook portion 2400 from the conduit retaining portion 2100, the component 2000 has a sensor mount 2200 configured to retain a patient sensor 29. When the component 2000 is hooked over another part of the patient interface which is worn by a patient, the sensor mount 2200 may be provided adjacent to or in contact with the patient's face. The hook portion 2400 may be repositionable along the side arm or headgear strap to provide the sensor mount 2200 at a desired location on the patient's face.

The patient sensor 29 may be either removably or irremovably retained by the sensor mount 2200.

The component 2000 of FIGS. 38A and 38B is shown with a sensor wire 2060 extending from the sensor mount 2200. In other arrangements, the patient sensor 29 may be provided with a power source and configured to communicate wirelessly with a processor and/or control circuitry, such a process or and/or control circuitry of a pulse oximeter.

The hook portion 2400 may be sized and configured to correspond to a part of the patient interface to which it is to be hooked over.

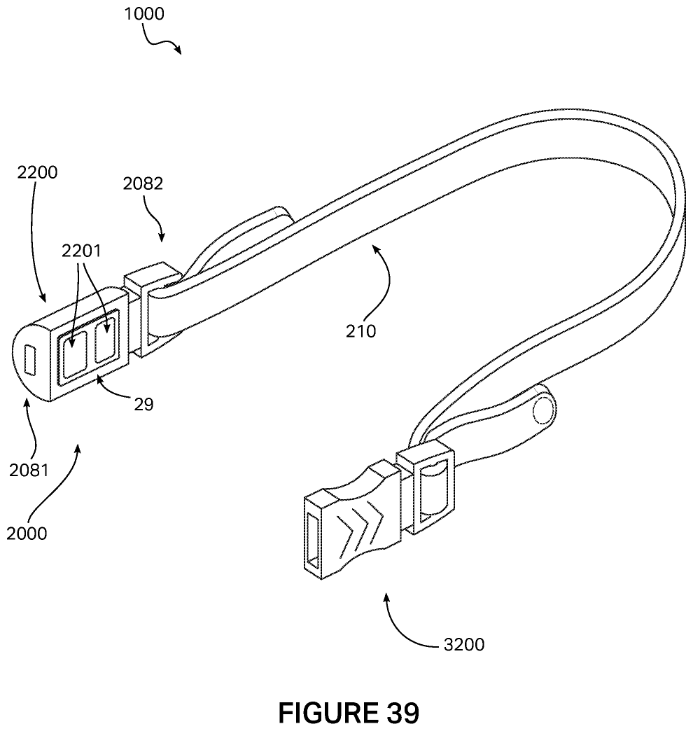

FIG. 39 illustrates another configuration of a component 2000 which is provided as part of a connector between two other parts of the patient respiratory interface 1000. The component 2000 has a first connector portion 2081 at which a side arm may be connected to the component 2000, and a second connector portion 2082 at which a headgear portion may be connected. As seen in FIG. 39, a headgear strap 210 is shown threaded through a buckle of the second connector portion 2082. Between the first connector portion 2081 and second connector portion 2082 is a sensor mount 2200 which is configured to retain a patient sensor 29. The patient sensor 29 may be exposed to the patient-facing side of the component 2000 through one or more openings 2201 of the sensor mount 2200.

As seen in FIG. 39, at the other end of the strap 210 a connector 3200 of the form previously described in relation to FIGS. 23-32 is provided. In other arrangements, the component 2000 may be provided at both ends of the strap 210.

Where a component 2000 of FIG. 39 is used as part of a patient respiratory interface, the component(s) and any connector 3200 may be disconnected from the side arms of the interface and reconnected with the opposite side arms in order to change the side of the patient's face at which the patient sensor 29 is located. Where it is not desirable to flip the headgear, this same functionality may be achieved by undoing the headgear strap 210 from the buckles of the component 2000 and connector 3200, switching the side arms with which each of the component 2000 and connector 3200 are associated, then reattaching the ends of the headgear strap 210 to the buckles.

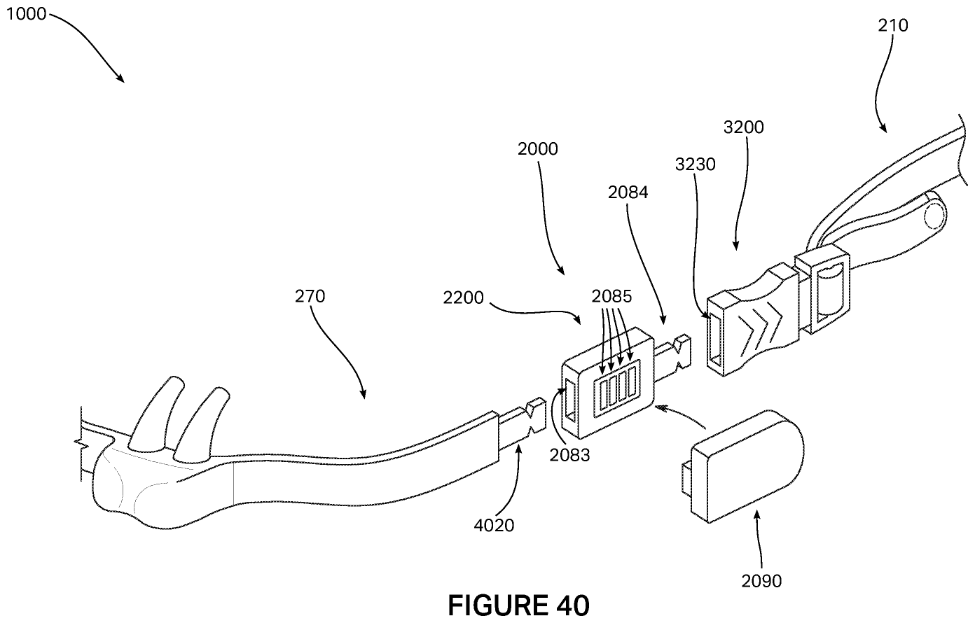

In some arrangements, a component 2000 which is configured to retain a patient sensor 29 may be provided as an intermediate component which is able to be inserted between two other parts of the patient interface. FIG. 40 illustrates an example of such an arrangement where a component 2000 is shown that is able to be inserted between a side arm 270 and connector 3200. Without the component 2000, the end 4020 of the side arm 270 may be insertable into the receiving portion 3230 of the connector 3200 to attach the two parts together.

The component 2000 of FIG. 40 has a first end 2083 which is configured to receive and couple with the end 4020 of the side arm 270. The component 2000 has a second end 2084 which is configured to be inserted into the receiving portion 3230 of the connector 3200. Between the first end 2083 and second end 2084, the component 2000 has a sensor mount 2200 at which a patient sensor may be retained.

While illustrated in FIG. 40 as having a respectively male to female connection between the end 4020 of the side arm 270 and first end 2083 of the component 2000, and a respectively male to female connection between the second end 2084 of the component 2000 and receiving portion 3230 of the connector 3200, it will be appreciated that the order of one or both sets of these connections may be rearranged as desired for a particular patient interface.

The component 2000 of FIG. 40 may be repositionable between either side of the patient interface for sensing at either side of the patient's face by disconnection of the component 2000 from the side arm 270 and connector 3200 at one side of the interface and reconnection at the other side of the interface.

A component 2000 described herein may be powered by a power source directly associated with the patient sensor 29, or may be powered by a remote power source. Similarly, a component 2000 may be configured to communicate by a wired or wireless connection with a processor and/or control circuitry. Where the patient sensor 29 is to communicate wirelessly, wireless communication circuitry and optionally a power source may be retained in the sensor mount 2200 along with the patient sensor. In other arrangements, wireless communication circuitry and a power source may be provided as part of a communication module.

A communication module 2090 is illustrated in FIG. 40. The communication module is for association with the sensor mount 2200 to enable wireless communication from the patient sensor 29 to a processor and/or other sensor control circuitry. As shown in FIG. 40, the component 2000 has ports 2085 at which the communication module 2090 is able to be connected to the component 2000 or the retained patient sensor 29.

It will be appreciated a communication module 2090 may be implemented with other forms of the component 2000 as described herein where wireless communication is desired for the patient sensor.

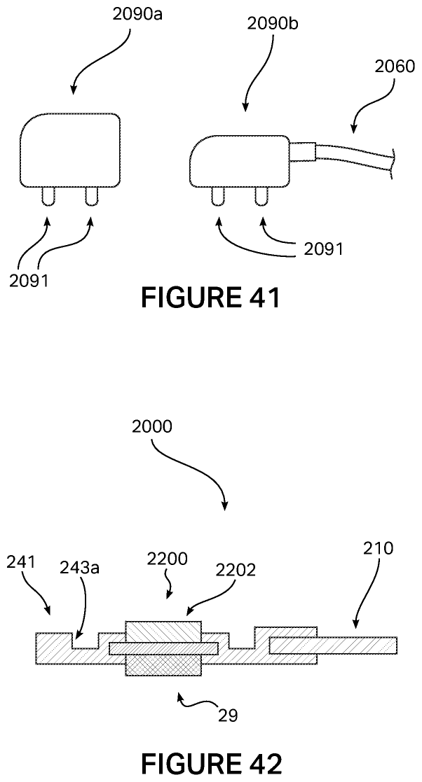

FIG. 41 illustrates two example configurations of a communication module, 2090a and 2090b. In the first configuration, the communications module 2090a includes a power source to power the communications module. The communications module 2090a is configured to wirelessly communicate sensor information from the patient sensor. In the second configuration, the communications module 2090b does not include a power source and is connected to a remote power source by a sensor wire 2060. The communications module 2090b may be arranged to wirelessly communicate sensor information or may transmit sensor information along the sensor wire 2060.

The communication modules 2090a and 2090b as illustrated in FIG. 41 each include prongs 2091 for interfacing with corresponding ports 2085 of the component 2000 for connection to the patient sensor 29.

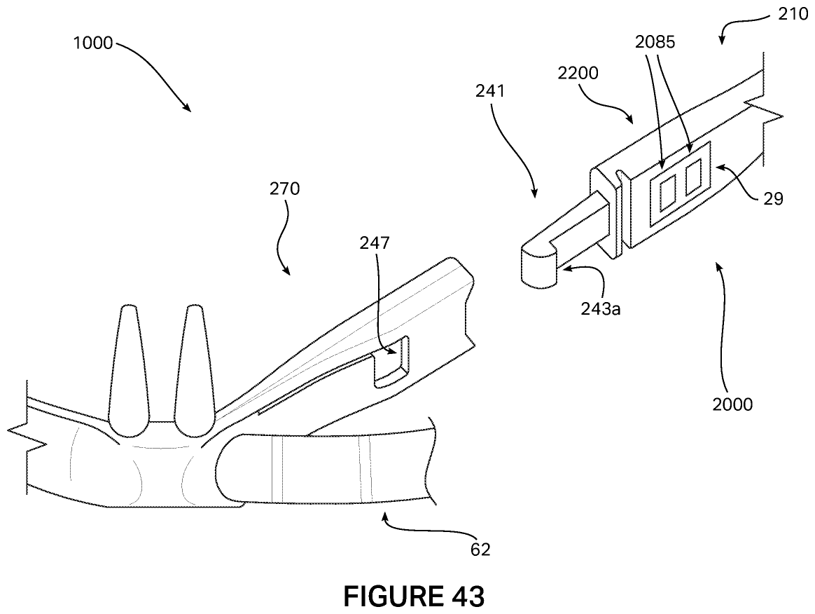

Configurations have been previously described, for example in relation to FIGS. 7C and 8B, where a patient sensor 29 may be retained by a side arm 270 which connects to one or both of the main body 110 or a headgear strap 210 by a connector in the form of the push fit clip 241 as particularly illustrated in FIG. 7F. FIG. 43 illustrates a further arrangement of a patient respiratory interface 1000 with a side arm 270 and headgear strap 210 which are connectable by a component 2000 that includes a push fit clip 241. The component 2000 is configured to retain a patient sensor 29. The headgear strap 210 may be associated with the component in any of the previously described ways, such as for example by a buckle or other part which retains the strap 210 like the toothed portion illustrated in the cross-sectional views of FIGS. 7G and 7H.

The push fit clip 241 is insertable within a channel of the side arm 270. When sufficiently inserted within the channel of the side arm 270, a withdrawal of the clip 241 from the channel may cause an engagement surface 243a of the clip 241 to engage a limit surface 247 of the side arm 270 and prevent the withdrawal of the clip 241 from the side arm 270. As illustrated in FIG. 43, the side arm 270 may have an opening so that a user can depress the end of the clip 241 to disengage the engagement surface 243a and limit surface 247 to allow separation of the headgear strap 210 and the side arm 270.

While illustrated in FIG. 43 as being having the push fit clip 241 associated with the headgear strap 210 and being insertable into a channel of the side arm 270, it will be appreciated that the association of the connector parts with the headgear strap 210 and side arm 270 may be reversed if desired.

As seen in FIG. 43, the component 2000 has a sensor mount 2200 for retaining a patient sensor 29. The sensor mount 2200 is configured to retain the patient sensor 29 and expose at least part of the patient sensor to the patient's face in use at a patient-facing side of the component 2000. In some arrangements, the sensor mount 2200 may be in the form of a sensor cavity 500 such as has been described previously, for example in relation to FIGS. 19 to 21. In other arrangements, a patient sensor 29 may be integrally provided as part of the component 2000. As seen in FIG. 43, the component 2000 may include ports 2085 for connection with a communication module 2090, as previously described.

FIG. 42 is a cross-sectional view of an illustrative configuration of a component 2000 for retaining a patient sensor 29 that includes a connector 241. The component 2000 of FIG. 42 has a sensor mount 2200 within which a patient sensor 29 is mounted. In the arrangement of FIG. 42, the patient sensor 29 is integrally formed with a remainder of the component 2000. For example, the patient sensor 29 may be at least partially overmolded into the component 2000. The integral forming of the patient sensor 29 is such that a portion of the patient sensor 29 is directly exposed at a patient facing side 2202 of the component 2000.

A sensor mount of a component according to the disclosure, such as a component 2000 as illustrated and described in relation to FIGS. 36A to 40 and 41, may include a sensor cavity, for example as previously described in relation to FIGS. 10A to 22. Where a component 2000 is powered and/or communicates by a wire, the sensor mount 2200 of a component 2000 according to the disclosure may include one or more wire cavities 700, for example as previously described in relation to FIGS. 10A to 22.

A securement feature as described herein, for example as previously described in relation to FIGS. 10A to 22, may generally encompass any disclosed features for attaching an accessory or component to another part of the patient respiratory interface. For example, the sensor mount 2200 of the component 2000 of FIG. 37 may be understood to be a securement feature for securing the component 2000 to the sensor aperture 2050 of the side arm 270. As a further example, the hook portion 2400 of the component 2000 of FIGS. 38A-B may be understood to be a securement feature for securing the component 2000 to the strap or side arm of the patient respiratory interface 1000.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1-218. (canceled)

219. A nasal interface cannula comprising:
a first prong and a second prong configured to provide an asymmetrical flow of gases at a patient's nares; and
a sensor configured to measure at least one patient parameter.

220. The nasal interface cannula of claim 219, wherein a difference between the first prong and the second prong is such as to cause the asymmetrical flow of gases at a patient's nares.

221. The nasal interface cannula of claim 219, wherein the first prong has a first shape and the second prong has a second shape.

222. The nasal interface cannula of claim 219, wherein the first prong has a smaller inner cross-sectional area in a direction transverse to gases flow through the first prong than a corresponding inner cross-sectional area of the second prong in a direction transverse to gases flow through the second prong.

223. The nasal interface cannula of claim 219, wherein the first prong and the second prong are configured such that at least about 60% of a total volumetric flow rate of gases flow dispensed by the nasal interface cannula is delivered out of the second prong.

224. The nasal interface cannula of claim 219, wherein a first prong cross-sectional area is smaller than a second prong cross-sectional area, wherein the first prong cross-sectional area and the second prong cross-sectional area are taken at corresponding locations on the first prong and second prong.

225. The nasal interface cannula of claim 224, wherein the first prong cross-sectional area and the second prong cross-sectional area is taken at respective proximal openings of the first prong and the second prong.

226. The nasal interface cannula of claim 219, wherein the at least one patient parameter measured by the sensor comprises at least one of heart rate, oxygen saturation, partial pressure of oxygen in blood, respiratory rate, partial pressure of CO₂ in blood, or blood oxygenation of a patient.

227. The nasal interface cannula of claim 219, wherein the sensor is positioned within a sensor cavity.

228. The nasal interface cannula of claim 227, wherein the sensor comprises a transducer, and/or a light source, wherein the sensor cavity is configured to orient the sensor such that the transducer and/or light source faces towards a patient

229. The nasal interface cannula of claim 228, wherein the transducer comprises at least one of a light transducer, an infrared transducer, or a red-light transducer.

230. The nasal interface cannula of claim 219, further comprising a flow source for providing a flow of gas.

231. A patient respiratory interface comprising:
a nasal cannula configured to provide an asymmetrical flow of gases at a patient's nares;
a gases inlet conduit configured to provide a flow of breathable gases to a patient through the nasal cannula; and
a sensor cavity;
a sensor positioned within the sensor cavity, the sensor configured to measure at least one patient parameter, wherein the at least one patient parameter measured by the sensor comprises at least one of heart rate, oxygen saturation, partial pressure of oxygen in blood, respiratory rate, partial pressure of CO₂ in blood, or blood oxygenation of the patient; and
wherein the sensor comprises a transducer and/or a light source,

232. The patient respiratory interface of claim 231 wherein the nasal cannula comprising a first prong and a second prong, wherein a difference between the first prong and the second prong is such as to cause the asymmetrical flow of gases at a patient's nares.

233. The patient respiratory interface of claim 232, wherein the first prong has a first shape and the second prong has a second shape.

234. The patient respiratory interface of claim 233, wherein the first prong has a smaller inner cross-sectional area in a direction transverse to gases flow through the first prong than a corresponding inner cross-sectional area of the second prong in a direction transverse to gases flow through the second prong.

235. The patient respiratory interface of claim 233, wherein the first prong and the second prong are configured such that at least about 60% of a total volumetric flow rate of gases flow dispensed by the nasal cannula is delivered out of the second prong.

236. The patient respiratory interface of claim 233, wherein a first prong cross-sectional area is smaller than a second prong cross-sectional area, wherein the first prong cross-sectional area and the second prong cross-sectional area are taken at corresponding locations on the first prong and second prong.

237. The patient respiratory interface of claim 236, wherein the first prong cross-sectional area and the second prong cross-sectional area is taken at respective proximal openings of the first prong and the second prong.

238. The patient respiratory interface of claim 232, wherein the sensor cavity is configured to orient the sensor such that the transducer and/or light source faces towards the patient
